SEQUENCE LISTING (45 sequences)

SEQ ID NO: 1 - DNA, 1662 bp, Homo sapiens
atggcggccccggcaggcggcggaggctccgcggtgtcggtgctggccccgaacggccggcgccacacggtgaaggtgacgccgagcaccgtgctgcttcaggttctggaggacacgtgccggcggcaggacttcaacccctgtgaatatgatctgaagtttcagaggagcgtgctcgacctttctctccagtggagatttgccaacctgcccaacaatgccaagctggagatggtgcccgcttcccggagccgtgaggggcctgagaacatggttcgcatcgctttgcagctggacgatggctcgaggttgcaggactctttctgttcaggccagaccctctgggagcttctcagccattttccacagatcagggagtgcctgcagcaccccggcggggccaccccagtctgcgtgtacacgagggatgaggtgacgggtgaagctgccctgcggggcacgacgctgcagtcgctgggcctgaccgggggcagcgccaccatcaggtttgtcatgaagtgctacgaccccgtgggcaagaccccaggaagcctgggctcgtcagcgtcggctggccaggcagccgccagcgctccacttcccttggaatctggggagctcagccgcggcgacttgagccgtccggaggacgcggacacctcagggccctgctgcgagcacactcaggagaagcagagcacaagggcacccgcagctgccccctttgttcctttctcgggtgggggacagagacaggggggccctcctgggcccacgaggcctctgacatcatcttcagctaagttgccgaagtccctctccagccctggaggcccctccaagccaaagaagtccaagtcgggccaggatccccagcaggagcaggagcaggagcgggagcgggatccccagcaggagcaggagcgggagcggcccgtggaccgggagcccgtggaccgggagccggtggtgtgccaccccgacctggaggagcggctgcaggcctggccagcggagctgcctgatgagttctttgagctgacggtggacgacgtgagaagacgcttggcccagctcaagagtgagcggaagcgcctggaagaagcccccttggtgaccaaggccttcagggaggcgcagataaaggagaagctggagcgctacccaaaggtggctctgagggtcctgttccccgaccgctacgtcctacagggcttcttccgccccagcgagacagtgggggacttgcgagacttcgtgaggagccacctggggaaccccgagctgtcattttacctgttcatcacccctccaaaaacagtcctggacgaccacacgcagaccctctttcaggcgaacctcttcccggccgctctggtgcacttgggagccgaggagccggcaggtgtctacctggagcctggcctgctggagcatgccatctccccatctgcggccgacgtgctggtggccaggtacatgtccagggccgccgggtccccttccccattgccagcccctgaccctgcacctaagtctgagccagctgctgaggagggggcgctggtcccccctgagcccatcccagggacggcccagcccgtgaagaggagcctgggcaaggtgcccaagtggctgaagctgccggccagcaagaggtga

SEQ ID NO: 2 - DNA, 2358 bp, Homo sapiens
atgctgccagcagcgccaggcaaggggcttgggagcccggaccccgccccctgcggcccagcgcccccaggaaatacaaaagatataataatgatatatgaagaagatgctgaggaatgggctctgtacttgacagaagtatttttacatgttgtgaaaagggaagccatcctgttatatcgcttggagaatttctcttttcggcatttggagttgctgaacttaacgtcttacaaatgtaaacttttgatattatcaaatagcctgcttagagacctaactccaaagaaatgtcagtttctggaaaagatacttcattcaccaaaaagtgtagttactttgctttgtggagtgaagagttcagatcagctctatgaattactaaatatctctcaaagcagatgggagatctcaactgaacaggaacctgaagactacatctctgtaatccagagtatcatattcaaagattctgaagactactttgaggtcaacattccaacagacctacgagcaaaacattctggggaaataagtgagagaaaggaaattgaagaactatcagaagcttcaagaaacaccataccactagcagtggtgcttcccactgaaattccatgtgagaatcctggtgaaatattcataattttgagagatgaagtaattggtgatactgtagaggttgaatttacatcaagtaataagcgcattagaacacggccagccctttggaataagaaagtctggtgcatgaaagctttagagtttcctgctggttcagtccatgtcaatgtctactgtgatggaatcgttaaagctacaaccaaaattaagtactacccaacagcaaaggcaaaggaatgcctattcagaatggcagattcaggagagagtttgtgccagaatagcattgaagaacttgatggtgtccttacatccatattcaaacatgagataccatattatgagttccagtctcttcaaactgaaatttgttctcaaaacaaatatactcatttcaaagaacttccaactcttctccactgtgcagcaaaatttggcttaaagaacctggctattcatttgcttcaatgttcaggagcaacctgggcatctaagatgaaaaatatggagggttcagaccccgcacatattgctgaaaggcatggtcacaaagaactcaagaaaatcttcgaagacttttcaatccaagaaattgacataaataatgagcaagaaaatgattatgaagaggatattgcctcattttccacatatattccttccacacagaacccagcatttcatcatgaaagcagaaagacatacgggcagagtgcagatggagctgaggcaaatgaaatggaaggggaaggaaaacagaatggatcaggcatggagaccaaacacagcccactagaggttggcagtgagagttctgaagaccagtatgatgacttgtatgtgttcattcctggtgctgatccagaaaataattcacaagagccactcatgagcagcagacctcctctccccccgccgcgacctgtagctaatgccttccaactggaaagacctcacttcaccttaccagggacaatggtggaaggccaaatggaaagaagtcaaaactggggtcatcctggtgttagacaagaaacaggagatgaacccaaaggagaaaaagagaagaaagaagaggaaaaagagcaggaggaggaagaagacccatatacttttgctgagattgatgacagtgaatatgacatgatattggccaatctgagtataaagaaaaaaactgggagtcggtctttcattataaatagacctcctgcccccacaccccgacccacaagtatacctccaaaagaggaaactacaccttacatagctcaagtgtttcaacaaaagacagccagaagacaatctgatgatgacaagttccgtggtcttcctaagaaacaagacagagctcggatagagagtccagccttttctactctcaggggctgtctaactgatggtcaggaagaactcatcctcctgcaggagaaagtaaagaatgggaaaatgtctatggatgaagctctggagaaatttaaacactggcagatgggaaaaagtggcctggaaatgattcagcaggagaaattacgacaactacgagactgcattattgggaaaaggccagaagaagaaaatgtctataataaactcaccattgtgcaccatccaggtggtaaggaaactgcccacaatgaaaataagttttataatgtacacttcagcaataagcttcctgctcgaccccaagttgaaaaggaatttggtttctgttgcaagaaagatcattaa

SEQ ID NO: 3 - DNA, 453 bp, Homo sapiens
atgagcaagatatcgcagcaaaacagcactccaggggtgaacggaattagtgttatccatacccaggcacatgccagcggcttacagcaggttcctcagctggtgcctgctggccctgggggaggaggcaaagctgtggctcccagcaagcagagcaaaaagagttcgcccatggatcgaaacagtgacgagtatcggcaacgccgagagaggaacaacatggctgtgaaaaagagccggttgaaaagcaagcagaaagcacaagacacactgcagagagtcaatcagctcaaagaagagaatgaacggttggaagcaaaaatcaaattgctgaccaaggaattaagtgtactcaaagatttgtttcttgagcatgcacacaaccttgcagacaacgtacagtccattagcactgaaaatacgacagcagatggcgacaatgcaggacagtag

SEQ ID NO: 4 - DNA, 1803 bp, Homo sapiens
atggagcatgtgacagagggctcctgggagtcgctgcctgtgccgctgcacccgcaggtgctgggcgcgctgcgggagctgggcttcccgtacatgacgccggtgcagtccgcaaccatccctctgttcatgcgaaacaaagatgtcgctgcagaagcggtcacaggtagtggcaaaacactcgcttttgtcatccccatcctggaaattcttctgagaagagaagagaagttaaaaaagagtcaggttggagccataatcatcacccccactcgagagctggccattcaaatagacgaggtcctgtcgcatttcacgaagcacttccccgagttcagccagattctttggatcggaggcaggaatcctggagaagatgttgagaggtttaagcaacaaggtgggaacatcattgtggccactccaggccgcttggaggacttgttccggaggaaggccgaaggcttggatctggccagctgtgtgcgatccctggatgtcctggtgttggatgaggcagacagacttctggacatggggtttgaggcaagcatcaacaccattctggagtttttgccaaagcagaggagaacaggccttttctctgccactcagacgcaggaagtggagaacctggtgagagcgggcctccggaaccctgtccgggtctcagtgaaggagaagggcgtggcagccagcagtgcccagaagaccccctcccgcctggaaaactactacatggtatgcaaggcagatgagaaatttaatcagctggtccattttcttcgcaatcataagcaggagaaacacctggtcttcttcagcacctgtgcctgtgtggaatactatgggaagactctggaagtgctggtgaagggcgtgaagattatgtgcattcacggaaagatgaaatataaacgcaataagatcttcatggagttccgcaaattgcaaagtgggattttagtgtgcactgatgtgatggcccggggaattgatattcctgaagtcaactgggttttgcagtatgaccctcccagcaatgcaagtgccttcgtgcatcgctgcggtcgcacagctcgcattggccacgggggcagcgctctggtgttcctcctgcccatggaagagtcatacatcaatttccttgcaattaaccaaaaatgccccctgcaggagatgaagccccagagaaacacagcggaccttctgccaaaactcaagtccatggccctggctgacagagctgtgtttgaaaagggcatgaaagcttttgtgtcatatgtccaagcttatgcaaagcatgaatgcaacctgattttcagattaaaggatcttgattttgccagccttgctcgaggttttgccctgctgaggatgcccaagatgccagaattgagagggaagcagtttccagattttgtgcccgtggacgttaataccgacacgattccatttaaagataaaatcagagaaaagcagaggcagaaactcctggagcaacaaagaagagagaaaacagaaaatgaagggagaagaaaattcataaaaaataaagcttggtcaaagcagaaggccaaaaaagaaaagaagaaaaaaatgaatgagaaaaggaaaagggaagagggttctgatattgaagatgaggacatggaagaacttcttaatgacacaagactcttgaaaaaacttaagaaaggcaaaataactgaagaagaatttgagaagggcttgttgacaactggcaaaagaacaatcaagacagtggatttagggatctcagatttggaagatggctgctaa

SEQ ID NO: 5 - DNA, 1191 bp, Homo sapiens
atggatcccagggggaccaagagaggagctgagaagacagaggtagctgagcctcggaacaaactacctcgtccagcaccttctctgcccacagaccctgccctctactctgggccctttcctttctaccggcgcccttcggaactgggctgcttctccctggatgctcaacgccagtaccatggagatgcccgagccctgcgctactatagcccaccccccactaacggtccaggccccaactttgacctcagagacggatacccggatcgataccagccccgggacgaggaggtccaggaaaggctggaccacctgctgtgctggctcctggaacaccgaggccggttggaggggggtccaggctggctggcagaggccatagtgacgtggcgggggcacctgacaaaactgctgacgacaccgtatgagcggcaggagggctggcagctggcagcctcccggttccagggaacactatacctgagtgaagtggagacaccgaacgctcgggcccagaggcttgctcggccaccgctcctccgggagcttatgtacatgggatacaaatttgagcagtacatgtgtgcagacaaacctggaagctccccagacccctctggggaggttaacaccaacgtggccttctgctctgtgctacgcagccgcctgggaagccaccctctgctcttctcaggggaggtagactgcacagacccccaagccccatccacacagcccccaacctgctatgtggagctcaagacctccaaggagatgcacagccctggccaatggaggagtttctacagacacaagctcctgaaatggtgggctcagtcattcctcccaggggtcccgaatgttgttgctggcttccgtaacccagacggttttgtctcttccctcaagacctttcctaccatgaagatgtttgaatatgtcaggaatgaccgtgacggctggaatccctctgtgtgcatgaacttctgtgccgccttccttagctttgcccagagcacggttgtccaggatgaccccaggctcgttcatctcttctcttgggagcctggcggcccagtcaccgtgtctgtacaccaagatgcaccttacgccttcctgcccatatggtatgtggaagctatgactcaggacctcccatcaccccccaagactccctctcccaaatag

SEQ ID NO: 6 - DNA, 2271 bp, Homo sapiens
atggacaatattaccaggcagaaccaattctacgatacccaagtcatcaaacaagaaaacgagtcaggctacgagaggagaccactggaaatggagcagcagcaggcctatcgtccagaaatgaagacagagatgaagcaaggagcacccaccagcttcctcccgcctgaagcttctcaactcaagccagacaggcagcaattccagagtcgaaagaggccttatgaagaaaaccggggacgggggtactttgagcaccgagaggataggaggggccgctctcctcagcctcctgctgaagaggatgaagatgactttgatgatacccttgttgctattgacacctataactgcgacctccacttcaaggtggcccgagatcggagtagtggctatccgctcacaattgagggctttgcatacctgtggtcaggagcccgtgccagctatggggtcagaaggggccgtgtatgcttcgagatgaagatcaatgaggaaatctccgtgaagcaccttccgtctacagagcctgacccccacgtggtccgtatcggctggtccctggactcctgcagcacccagctaggcgaagagcctttctcctatggctatggaggcactgggaagaagtccaccaatagccggtttgaaaactacggagacaagtttgcagagaacgatgtgattggctgctttgcggattttgaatgtggaaatgacgtggaactgtcttttaccaagaatggaaagtggatgggcattgctttccgaatccagaaggaagccttggggggtcaggccctctatcctcatgtcctggtgaagaattgcgcagtggagttcaacttcggacagagagcagagccctactgttctgtcctcccggggtttaccttcatccagcaccttccccttagtgagcgtatccggggcaccgttggaccaaagagcaaggcagaatgtgagattctgatgatggtgggcctgcctgctgctggcaagaccacatgggccatcaaacatgcagcctccaacccttccaagaagtacaacatcctgggtaccaatgccatcatggataagatgcgggtgatgggcctacgccggcagcggaactatgctggccgctgggatgtcctgatccagcaggccacccagtgcctcaaccgcctcatccagattgctgcccgcaagaaacgcaactatatcctagatcagacaaatgtttatgggtcagcccagagacgaaaaatgagaccatttgaaggcttccagcgcaaagctattgtaatttgtcccactgacgaggacctaaaagaccgaacaataaagcgaaccgacgaggaagggaaggatgtcccagatcatgcggtcttagaaatgaaagccaacttcacgttgccagatgttggggacttcctggatgaggttctgttcattgagctgcagcgggaggaagcggacaagctagtgaggcagtacaacgaggaaggccgcaaggctgggccaccccctgaaaagcgctttgacaaccgaggtggtggtggcttccggggccgcgggggtggtggtggcttccagcgctatgaaaaccgaggaccccctggaggcaaccgtggcggcttccagaaccgagggggaggcagcggtggaggaggcaactaccgaggaggtttcaaccgcagcggaggtggtggctatagccagaaccgctggggtaacaacaaccgggataacaacaactccaacaacagaggcagctacaaccgggctccccagcaacagccgccaccacagcagcctccgccaccacagccaccaccccagcagccaccgccaccacccagctacagccctgctcggaaccccccaggggccagcacctacaataagaacagcaacatccctggctcaagcgccaataccagcacccccaccgtcagcagctacagccctccacagccgagttacagccagccaccctacaaccagggaggttacagccagggctacacagccccaccgcctccacctccaccaccacctgcctacaactatgggagctacggcggttacaacccggccccctataccccaccgccaccccccaccgcacagacctaccctcagcccagctataaccagtatcagcagtatgcccagcagtggaaccagtactatcagaaccagggccagtggccgccatactacgggaactacgactacgggagctactccgggaacacacagggtggcacaagtacacagtag

SEQ ID NO: 7 - DNA, 1581 bp, Homo sapiens
atggcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacacaggctatggaactcagtcaactccccagggatatggctcgactggcggctatggcagtagccagagctcccaatcgtcttacgggcagcagtcctcctatcctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtggaggccgcggcaggggtggcagtggtggcggcggcggcggcggcggtggtggttacaaccgcagcagtggtggctatgaacccagaggtcgtggaggtggccgtggaggcagaggtggcatgggcggaagtgaccgtggtggcttcaataaatttggtggccctcgggaccaaggatcacgtcatgactccgaacaggataattcagacaacaacaccatctttgtgcaaggcctgggtgagaatgttacaattgagtctgtggctgattacttcaagcagattggtattattaagacaaacaagaaaacgggacagcccatgattaatttgtacacagacagggaaactggcaagctgaagggagaggcaacggtctcttttgatgacccaccttcagctaaagcagctattgactggtttgatggtaaagaattctccggaaatcctatcaaggtctcatttgctactcgccgggcagactttaatcggggtggtggcaatggtcgtggaggccgagggcgaggaggacccatgggccgtggaggctatggaggtggtggcagtggtggtggtggccgaggaggatttcccagtggaggtggtggcggtggaggacagcagcgagctggtgactggaagtgtcctaatcccacctgtgagaatatgaacttctcttggaggaatgaatgcaaccagtgtaaggcccctaaaccagatggcccaggagggggaccaggtggctctcacatggggggtaactacggggatgatcgtcgtggtggcagaggaggctatgatcgaggcggctaccggggccgcggcggggaccgtggaggcttccgagggggccggggtggtggggacagaggtggctttggccctggcaagatggattccaggggtgagcacagacaggatcgcagggagaggccgtattaa

SEQ ID NO: 8 - DNA, 783 bp, Homo sapiens
atgaaggtagaggtgctgcctgccctgaccgacaactacatgtacctggtcattgatgatgagaccaaggaggctgccattgtggatccggtgcagccccagaaggtcgtggacgcggcgagaaagcacggggtgaaactgaccacagtgctcaccacccaccaccactgggaccatgctggcgggaatgagaaactggtcaagctggagtcgggactgaaggtgtacgggggtgacgaccgtatcggggccctgactcacaagatcactcacctgtctacactgcaggtggggtctctgaacgtcaagtgcctggcgaccccgtgccacacttcaggacacatttgttacttcgtgagcaagcccggaggctcggagccccctgccgtgttcacaggtgacaccttgtttgtggctggctgcgggaagttctatgaagggactgcggatgagatgtgtaaagctctgctggaggtcttgggccggctccccccggacacaagagtctactgtggccacgagtacaccatcaacaacctcaagtttgcacgccacgtggagcccggcaatgccgccatccgggagaagctggcctgggccaaggagaagtacagcatcggggagcccacagtgccatccaccctggcagaggagtttacctacaaccccttcatgagagtgagggagaagacggtgcagcagcacgcaggtgagacggacccggtgaccaccatgcgggccgtgcgcagggagaaggaccagttcaagatgccccgggactga

SEQ ID NO: 9 - DNA, 954 bp, Homo sapiens
atgtcccacggccccaagcagcccggcgcggccgccgcgccggcgggcggcaaggctccgggccagcatgggggcttcgtggtgactgtcaagcaagagcgcggcgagggtccacgcgcgggcgagaaggggtcccacgaggaggagccggtgaagaaacgcggctggcccaagggcaagaagcggaagaagattctgccgaatgggcccaaggcaccggtcacgggctacgtgcgcttcctgaacgagcggcgcgagcagatccgcacgcgccacccggatctgccctttcccgagatcaccaagatgctgggcgccgagtggagcaagctgcagccaacggaaaagcagcggtacctggatgaggccgagagagagaagcagcagtacatgaaggagctgcgggcgtaccagcagtctgaagcctataagatgtgcacggagaagatccaggagaagaagatcaagaaagaagactcgagctctgggctcatgaacactctcctgaatggacacaagggtggggactgcgatggcttctccaccttcgatgttcccatcttcactgaagagttcttggaccaaaacaaagcgcgtgaggcggagcttcggcgcttgcggaagatgaatgtggccttcgaggagcagaacgcggtactgcagaggcacacgcagagcatgagcagcgcgcgcgagcgtctggagcaggagctggcgctggaggagcggaggacgctggcgctgcagcagcagctccaggccgtgcgccaggcgctcaccgccagcttcgcctcactgccggtgccgggcacgggcgaaacgcccacgctgggcactctggacttctacatggcccggcttcacggagccatcgagcgcgaccccgcccagcacgagaagctcatcgtccgcatcaaggaaatcctggcccaggtcgccagcgagcacctgtga

SEQ ID NO: 10 - DNA, 675 bp, Homo sapiens
atgagttcctatttcgtgaactccaccttccccgtcactctggccagcgggcaggagtccttcctgggccagctaccgctctattcgtcgggctatgcggacccgctgagacattaccccgcgccctacgggccagggccgggccaggacaagggctttgccacttcctcctattacccgccggcgggcggtggctacggccgagcggcgccctgcgactacgggccggcgccggccttctaccgcgagaaagagtcggcctgcgcactctccggcgccgacgagcagcccccgttccaccccgagccgcggaagtcggactgcgcgcaggacaagagcgtgttcggcgagacagaagagcagaagtgctccactccggtctacccgtggatgcagcggatgaattcgtgcaacagttcctcctttgggcccagcggccggcgaggccgccagacatacacacgttaccagacgctggagctggagaaggagtttcactacaatcgctacctgacgcggcggcggcgcatcgagatcgcgcacgccctgtgcctgacggagaggcagatcaagatatggttccagaaccgacgcatgaagtggaaaaaggagagcaaactgctcagcgcgtctcagctcagtgccgaggaggaggaagaaaaacaggccgagtga

SEQ ID NO: 11 - DNA, 2190 bp, Homo sapiens
atgggaaaaaaatacaagaacattgttctactaaaaggattagaggtcatcaatgattatcattttagaatggttaagtccttactgagcaacgatttaaaacttaatttaaaaatgagagaagagtatgacaaaattcagattgctgacttgatggaagaaaagttccgaggtgatgctggtttgggcaaactaataaaaattttcgaagatataccaacgcttgaagacctggctgaaactcttaaaaaagaaaagttaaaagtaaaaggaccagccctatcaagaaagaggaagaaggaagtggatgctacttcacctgcaccctccacaagcagcactgtcaaaactgaaggagcagaggcaactcctggagctcagaaaagaaaaaaatcaaccaaagaaaaggctggacccaaagggagtaaggtgtccgaggaacagactcagcctccctctcctgcaggagccggcatgtccacagccatgggccgttccccatctcccaagacctcattgtcagctccacccaacacttcttcaactgagaacccgaaaacagtggccaaatgtcaggtaactcccagaagaaatgttctccaaaaacgcccagtgatagtgaaggtactgagtacaacaaagccatttgaatatgagaccccagaaatggagaaaaaaataatgtttcatgctacagtggctacacagacacagttcttccatgtgaaggttttaaacaccagcttgaaggagaaattcaatggaaagaaaatcatcatcatatcagattatttggaatatgatagtctcctagaggtcaatgaagaatctactgtatctgaagctggtcctaaccaaacgtttgaggttccaaataaaatcatcaacagagcaaaggaaactctgaagattgatattcttcacaaacaagcttcaggaaatattgtatatggggtatttatgctacataagaaaacagtaaatcagaagaccacaatctacgaaattcaggatgatagaggaaaaatggatgtagtggggacaggacaatgtcacaatatcccctgtgaagaaggagataagctccaacttttctgctttcgacttagaaaaaagaaccagatgtcaaaactgatttcagaaatgcatagttttatccagataaagaaaaaaacaaacccgagaaacaatgaccccaagagcatgaagctaccccaggaacagagtcagcttccaaatccttcagaggccagcacaaccttccctgagagccatcttcggactcctcagatgccaccaacaactccatccagcagtttcttcaccaagaaaagtgaagacacaatctccaaaatgaatgacttcatgaggatgcagatactgaaggaagggagtcattttccaggaccgttcatgaccagcataggcccagctgagagccatccccacactcctcagatgcctccatcaacaccaagcagcagtttcttaaccacgttgaaaccaagactgaagactgaacctgaagaagtttccatagaagacagtgcccagagtgacctcaaagaagtgatggtgctgaacgcaacagaatcatttgtatatgagcccaaagagcagaagaaaatgtttcatgccacagtggcaactgagaatgaagtcttccgagtgaaggtttttaatattgacctaaaggagaagttcaccccaaagaagatcattgccatagcaaattatgtttgccgcaatgggttcctggaggtatatcctttcacacttgtggctgatgtgaatgctgaccgaaacatggagatcccaaaaggattgattagaagtgccagcgtaactcctaaaatcaatcagctttgctcacaaactaaaggaagttttgtgaatggggtgtttgaggtacataagaaaaatgtaaggggtgaattcacttattatgaaatacaagataatacagggaagatggaagtggtggtgcatggacgactgaccacaatcaactgtgaggaaggagataaactgaaactcacctgctttgaattggcaccgaaaagtgggaataccggggagttgagatctgtaattcatagtcacatcaaggtcatcaagaccaggaaaaacaagaaagacatactcaatcctgattcaagtatggaaacttcaccagactttttcttctaa

SEQ ID NO: 12 - DNA, 840 bp, Homo sapiens
atgaacaaggtagagctggagtctcgcctggaagggctgaccgacgagatcaacttcctcaggcagctatatgaagaggagatccgggagctgcagtcccagatctcggacacatctgtggtgctgtccatggacaacagccgctccctggacatggacagcatcattgctgaggtcaaggcacagtacgaggatattgccaaccgcagccgggctgaggctgagagcatgtaccagatcaagtatgaggagctgcagagcctggctgggaagcacggggatgacctgcggcgcacaaagactgagatctctgagatgaaccggaacatcagccggctccaggctgagattgagggcctcaaaggccagagggcttccctggaggccgccattgcagatgccgagcagcgtggagagctggccattaaggatgccaacgccaagttgtccgagctggaggccgccctgcagcgggccaagcaggacatggcgcggcagctgcgtgagtaccaggagctgatgaacgtcaagctggccctggacatcgagatcgccacctacaggaagctgctggagggcgaggagagccggctggagtctgggatgcagaacatgagtattcatacgaagaccaccagcggctatgcaggtggtctgagctcggcctatgggggcctcacaagccccggcctcagctacagcctgggctccagctttggctctggcgcgggctccagctccttcagccgcaccagctcctccagggccgtggttgtgaagaagatcgagacacgtgatgggaagctggtgtctgagtcctctgacgtcctgcccaagtga

SEQ ID NO: 13 - DNA, 630 bp, Homo sapiens
atgggctccgtgtccaaccagcagtttgcaggtggctgcgccaaggcggcagaagaggcgcccgaggaggcgccggaggacgcggcccgggcggcggacgagcctcagctgctgcacggtgcgggcatctgtaagtggttcaacgtgcgcatggggttcggcttcctgtccatgaccgcccgcgccggggtcgcgctcgaccccccagtggatgtctttgtgcaccagagtaagctgcacatggaagggttccggagcttgaaggagggtgaggcagtggagttcacctttaagaagtcagccaagggtctggaatccatccgtgtcaccggacctggtggagtattctgtattgggagtgagaggcggccaaaaggaaagagcatgcagaagcgcagatcaaaaggagacaggtgctacaactgtggaggtctagatcatcatgccaaggaatgcaagctgccaccccagcccaagaagtgccacttctgccagagcatcagccatatggtagcctcatgtccgctgaaggcccagcagggccctagtgcacagggaaagccaacctactttcgagaggaagaagaagaaatccacagccctaccctgctcccggaggcacagaattga

SEQ ID NO: 14 - DNA, 2187 bp, Homo sapiens
atgaaccagccagagtctgccaacgatcctgaacccctgtgtgcagtgtgtggccaagcccactccttggaggaaaaccacttctacagctatccagaggaagtggatgatgacctcatctgccacatctgcctgcaggctttgctggaccccctggacactccgtgtggacacacctactgcaccctctgcctcaccaacttcctggtggagaaggacttctgtcccatggaccgcaagcctctggttctgcagcactgcaagaagtccagcatcctggtcaacaaactcctcaacaagctactggtgacctgcccattcagggagcactgcacccaggtgttgcagcgctgtgacctcgagcatcactttcaaaccagctgtaaaggtgcctcccactacggcctgaccaaagataggaagaggcgctcacaagatggctgtccagacggctgtgcgagcctcacagccacggctccctccccagaggtttctgcagctgccaccatctccttaatgacagacgagcctggcctagacaaccctgcctacgtgtcctcggcagaggacgggcagccagcaatcagcccagtggactctggccggagcaaccgaactagggcacggccctttgagagatccactattagaagcagatcatttaaaaaaataaatcgagctttgagtgttcttcgaaggacaaagagcgggagtgcagttgccaaccatgccgaccagggcagggaaaattctgaaaacaccactgcccctgaagtctttccaaggttgtaccacctgattccagatggtgaaattaccagcatcaagatcaatcgagtagatcccagtgaaagcctctctattaggctggtgggaggtagcgaaaccccactggtccatatcattatccaacacatttatcgtgatggggtgatcgccagagacggccggctactgccaggagacatcattctaaaggtcaacgggatggacatcagcaatgtccctcacaactacgctgtgcgtctcctgcggcagccctgccaggtgctgtggctgactgtgatgcgtgaacagaagttccgcagcaggaacaatggacaggccccggatgcctacagaccccgagatgacagctttcatgtgattctcaacaaaagtagccccgaggagcagcttggaataaaactggtgcgcaaggtggatgagcctggggttttcatcttcaatgtgctggatggcggtgtggcatatcgacatggtcagcttgaggagaatgaccgtgtgttagccatcaatggacatgatcttcgatatggcagcccagaaagtgcggctcatctgattcaggccagtgaaagacgtgttcacctcgtcgtgtcccgccaggttcggcagcggagccctgacatctttcaggaagccggctggaacagcaatggcagctggtccccagggccaggggagaggagcaacactcccaagcccctccatcctacaattacttgtcatgagaaggtggtaaatatccaaaaagaccccggtgaatctctcggcatgaccgtcgcagggggagcatcacatagagaatgggatttgcctatctatgtcatcagtgttgagcccggaggagtcataagcagagatggaagaataaaaacaggtgacattttgttgaatgtggatggggtcgaactgacagaggtcagccggagtgaggcagtggcattattgaaaagaacatcatcctcgatagtactcaaagctttggaagtcaaagagtatgagccccaggaagactgcagcagcccagcagccctggactccaaccacaacatggccccacccagtgactggtccccatcctgggtcatgtggctggaattaccacggtgcttgtataactgtaaagatattgtattacgaagaaacacagctggaagtctgggcttctgcattgtaggaggttatgaagaatacaatggaaacaaaccttttttcatcaaatccattgttgaaggaacaccagcatacaatgatggaagaattagatgtggtgatattcttcttgctgtcaatggtagaagtacatcaggaatgatacatgcttgcttggcaagactgctgaaagaacttaaaggaagaattactctaactattgtttcttggcctggcacttttttatag

SEQ ID NO: 15 - DNA, 2019 bp, Homo sapiens
atgggcgtgggccggctggacatgtatgtgctgcacccgccctccgccggcgccgagcgcacgctggcctgtgtgtgcgccctgctggtgtggcaccccgccggccccggcgagaaggtggtgcgcgtgctgttccccggttgcaccccgcccgcctgcctcctggacggcctggtccgcctgcagcacttgaggttcctgcgagagcccgtggtgacgccccaggacctggaggggccggggcgagccgagagcaaagagagcgtgggctcccgggacagctcgaagagagagggcctcctggccacccaccctagacctggccaggagcgccctggggtggcccgcaaggagccagcacgggctgaggccccacgcaagactgagaaagaagccaagaccccccgggagttgaagaaagaccccaaaccgagtgtctcccggacccagccgcgggaggtgcgccgggcagcctcttctgtgcccaacctcaagaagacgaatgcccaggcggcacccaagccccgcaaagcgcccagcacgtcccactctggcttcccgccggtggcaaatggaccccgcagcccgcccagcctccgatgtggagaagccagcccccccagtgcggcctgcggctctccggcctcccagctggtggccacgcccagcctggagctggggccgatcccagccggggaggagaaggcactggagctgcctttggccgccagctcaatcccaaggccacgcacaccctcccctgagtcccaccggagccccgcagagggcagcgagcggctgtcgctgagcccactgcggggcggggaggccgggccagacgcctcacccacagtgaccacacccacggtgaccacgccctcactacccgcagaggtgggctccccgcactcgaccgaggtggacgagtccctgtcggtgtcctttgagcaggtgctgccgccatccgcccccaccagtgaggctgggctgagcctcccgctgcgtggcccccgggcgcggcgctcggcttccccacacgatgtggacctgtgcctggtgtcaccctgtgaatttgagcatcgcaaggcggtgccaatggcaccggcacctgcgtcccccggcagctcgaatgacagcagtgcccggtcacaggaacgggcaggtgggctgggggccgaggagacgccacccacatcggtcagcgagtccctgcccaccctgtctgactcggatcccgtgcccctggcccccggtgcggcagactcagacgaagacacagagggctttggagtccctcgccacgaccctttgcctgaccccctcaaggtccccccaccactgcctgacccatccagcatctgcatggtggaccccgagatgctgccccccaagacagcacggcaaacggagaacgtcagccgcacccggaagcccctggcccgccccaactcacgcgctgccgcccccaaagccactccagtggctgctgccaaaaccaaggggcttgctggtggggaccgtgccagccgaccactcagtgcccggagtgagcccagtgagaagggaggccgggcacccctgtccagaaagtcctcaacccccaagactgccactcgaggcccgtcggggtcagccagcagccggcccggggtgtcagccaccccacccaagtccccggtctacctggacctggcctacctgcccagcgggagcagcgcccacctggtggatgaggagttcttccagcgcgtgcgcgcgctctgctacgtcatcagtggccaggaccagcgcaaggaggaaggcatgcgggccgtcctggacgcgctactggccagcaagcagcattgggaccgtgacctgcaggtgaccctgatccccactttcgactcggtggccatgcatacgtggtacgcagagacgcacgcccggcaccaggcgctgggcatcacggtgttgggcagcaacagcatggtgtccatgcaggatgacgccttcccggcctgcaaggtggagttctag

SEQ ID NO: 16 - DNA, 1422 bp, Homo sapiens
atggctgccccgtgtttgctgcggcaaggacgagccggggcgctgaagactatgctccaggaagcccaggtgtttcgaggacttgcttctacggtttctttgtctgcggaatcagggaagagtgaaaagggtcagccacagaattccaagaagcaaagtccaccaaaaaatgtagtggaaccaaaggagaggggcaagctcctagccacccagacagcagctgaattgtctaaaaacttatcttcacccagttcttacccgccagctgtgaataagggcaggaaggtagctagtcccagtcccagtggcagcgtgctattcacagatgaaggggttccgaaatttttgtcaagaaagactttggtagagtttccacagaaagttctgtctccattcagaaaacagggctctgattcagaagctcgtcaggtgggtcggaaagtgacgtcgccttcgtcttcatcctcgtccagctcctctgattctgaatctgatgatgaggctgacgtttcagaggtcactcctcgagtggtgagcaaaggcagaggggggcttcgaaaaccagaggcctctcattcctttgaaaacagagccccccgagttacagtatcagcaaaagagaaaaccttgctgcagaagccgcatgtggacattactgatccagagaagccccaccagccaaagaagaaagggtcccctgctaagccatcagaaggcagggaaaatgcgagaccaaaaaccacaatgcccagatctcaagtagatgaagagtttttgaagcaaagtttaaaggaaaaacaattgcagaaaacatttagattaaatgaaatagataaagaaagccaaaagccatttgaagttaaaggacccttacctgtccacacaaaatcagggttgtctgcgccaccgaagggcagcccagcgcctgctgtgttggcagaagaggccagagcagaggggcagctgcaagccagtcctcctggggcggcagaggggcatctggaaaaacccgtgccagagccccagcgcaaggcggcccctcccctgcccagaaaggaaacctcagggacgcagggaatagaaggccacctgaagggtggacaggcaatcgtggaagatcagataccaccaagcaatttggagacagttcctgttgagaataaccacggtttccatgaaaagacagcagcgctgaagcttgaggccgagggcgaggccatggaagatgcagccgcgccagggaacgaccgaggcggcacacaggagccagccccagtgcctgctgagccgtttgacaacactacctacaagaacctgcagcatcatgactacagcacgtacaccttcttagacctcaacctcgaactctcaaaattcaggatgcctcagccctcctcaggccgggagtcacctcgacactga

SEQ ID NO: 17 - DNA, 1911 bp, Homo sapiens
atgaaccccagtgcccccagctaccccatggcctcgctctacgtgggggacctccaccccgacgtgaccgaggcgatgctctacgagaagttcagcccggccgggcccatcctctccatccgggtctgcagggacatgatcacccgccgctccttgggctacgcgtatgtgaacttccagcagccggcggacgcggagcgtgctttggacaccatgaattttgatgttataaagggcaagccagtacgcatcatgtggtctcagcgtgatccatcacttcgcaaaagtggagtaggcaacatattcattaaaaatctggacaaatccattgataataaagcactgtatgatacattttctgcttttggtaacatcctttcatgtaaggtggtttgtgatgaaaatggttccaagggctatggatttgtacactttgagacgcaggaagcagctgaaagagctattgaaaaaatgaatggaatgctcctaaatgatcgcaaagtatttgttggacgatttaagtctcgtaaagaacgagaagctgaacttggagctagggcaaaagaattcaccaatgtttacatcaagaattttggagaagacatggatgatgagcgccttaaggatctctttggcaagtttgggcctgccttaagtgtgaaagtaatgactgatgaaagtggaaaatccaaaggatttggatttgtaagctttgaaaggcatgaagatgcacagaaagctgtggatgagatgaacggaaaggagctcaatggaaaacaaatttatgttggtcgagctcagaaaaaggtggaacggcagacggaacttaagcgcaaatttgaacagatgaaacaagataggatcaccagataccagggtgttaatctttatgtgaaaaatcttgatgatggtattgatgatgaacgtctccggaaagagttttctccatttggtacaatcactagtgcaaaggttatgatggagggtggtcgcagcaaagggtttggttttgtatgtttctcctccccagaagaagccactaaagcagttacagaaatgaacggtagaattgtggccacaaagccattgtatgtagctttagctcagcgcaaagaagagcgccaggctcacctcactaaccagtatatgcagagaatggcaagtgtacgagctgttcccaaccctgtaatcaacccctaccagccagcacctccttcaggttacttcatggcagctatcccacagactcagaaccgtgctgcatactatcctcctagccaaattgctcaactaagaccaagtcctcgctggactgctcagggtgccagacctcatccattccaaaatatgcccggtgctatccgcccagctgctcctagaccaccatttagtactatgagaccagcttcttcacaggttccacgagtcatgtcaacacagcgtgttgctaacacatcaacacagacaatgggtccacgtcctgcagctgcagccgctgcagctactcctgctgtccgcaccgttccacagtataaatatgctgcaggagttcgcaatcctcagcaacatcttaatgcacagccacaagttacaatgcaacagcctgctgttcatgtacaaggtcaggaacctttgactgcttccatgttggcatctgcccctcctcaagagcaaaagcaaatgttgggtgaacggctgtttcctcttattcaagccatgcaccctactcttgctggtaaaatcactggcatgttgttggagattgataattcagaacttcttcatatgctcgagtctccagagtcactccgttctaaggttgatgaagctgtagctgtactacaagcccaccaagctaaagaggctgcccagaaagcagttaacagtgccaccggtgttccaactgtttaa

SEQ ID NO: 18 - DNA, 780 bp, Homo sapiens
atgctggagagtagcggctgcaaagcgctgaaggagggcgtgctggagaagcgcagcgacgggttgttgcagctctggaagaaaaagtgttgcatcctcaccgaggaagggctgctgcttatcccgcccaagcagctgcaacaccagcagcagcagcaacagcagcagcagcagcagcaacaacagcccgggcaggggccggccgagccgtcccaacccagtggccccgctgtcgccagcctcgagccgccggtcaagctcaaggaactgcacttctccaacatgaagaccgtggactgtgtggagcgcaagggcaagtacatgtacttcactgtggtgatggcagagggcaaggagatcgactttcggtgcccgcaagaccagggctggaacgccgagatcacgctgcagatggtgcagtacaagaatcgtcaggccatcctggcggtcaaatccacgcggcagaagcagcagcacctggtccagcagcagcccccctcgcagccgcagccgcagccgcagctccagccccaaccccagcctcagcctcagccgcaaccccagccccaatcacaaccccagcctcagccccaacccaagcctcagccccagcagctccacccgtatccgcatccacatccacatccacactctcatcctcactcgcacccacaccctcacccgcacccgcatccgcaccaaataccgcacccacacccacagccgcactcgcagccgcacgggcaccggcttctccgcagcacctccaactctgcctga

SEQ ID NO: 19 - DNA, 1719 bp, Homo sapiens
atggcggatttcgaagagttgaggaatatggtttctagttttagggtttctgaactacaagtattactaggctttgctggacggaataaaagtggacgcaagcatgacctcctgatgagggcgctgcatttattgaagagcggctgcagccctgcggttcagattaaaatccgagaattgtatagacgccgatatccacgaactcttgaaggactttctgatttatccacaatcaaatcatcggttttcagtttggatggtggctcatcacctgtagaacctgacttggccgtggctggaatccactcgttgccttccacttcagttacacctcactcaccatcctctcctgttggttctgtgctgcttcaagatactaagcccacatttgagatgcagcagccatctcccccaattcctcctgtccatcctgatgtgcagttaaaaaatctgcccttttatgatgtccttgatgttctcatcaagcccacgagtttagttcaaagcagtattcagcgatttcaagagaagttttttatttttgctttgacacctcaacaagttagagagatatgcatatccagggattttttgccaggtggtaggagagattatacagtccaagttcagttgagactttgcctggcagagacaagttgccctcaagaagataactatccaaatagtctatgtataaaagtaaatgggaagctatttcctttgcctggctatgcaccaccgcctaaaaatgggattgaacagaagcgccctggacgccccttgaatattacatctttagttaggttatcttcagctgtgccaaaccaaatttccatttcttgggcatcagaaattgggaagaattactctatgtctgtatatcttgtacggcagcttacatcagccatgttattacagagattaaaaatgaaaggtattagaaaccctgatcattccagagcactaattaaagaaaaacttactgcagatcctgatagtgaaattgctacaactagccttcgggtatccttgatgtgccctttaggaaaaatgaggctgacaatcccatgccgtgcagtgacttgtacacatctgcagtgttttgatgctgccctctatctacaaatgaatgagaaaaagcccacctggatttgtcctgtgtgtgacaaaaaagctgcctatgaaagtctaatattagatgggctttttatggaaattctcaatgactgttctgatgtagatgagatcaaattccaagaagatggttcttggtgtccaatgagaccgaagaaagaagctatgaaagtatccagccaaccgtgtacaaaaatagaaagttcaagcgtcctcagtaagccttgttcagtgactgtagccagtgaggcaagcaagaagaaagtagatgttattgatcttacaatagaaagctcttctgacgaagaggaagaccctcctgccaaaaggaaatgcatctttatgtcagaaacacaaagcagcccaaccaaaggggttctcatgtatcagccatcttctgtaagggtgcccagtgtgacttcggttgatcctgctgctattccgccttcattaacagactactcagtaccattccaccatacgccaatatcaagcatgtcatcagatttgccaggagaacaaagaagaaatgatattaataatgaactgaagcttggaacatcttctgatactgtgcaacagtga

SEQ ID NO: 20 - DNA, 3072 bp, Homo sapiens
atgacaatgctcaccattgaacagttatcctacctgctcaagtttgccattcagaaaatgaaacagccagggacagatgcattccagaagcccgttccattggaacagcaccctgactatgcggaatacatcttccatccaatggacctttgtacattggaaaagaatgcgaaaaagaaaatgtatggctgcacagaagccttcctggctgatgcaaagtggattttgcacaactgcatcatttataatgggggaaatcacaaattgacgcaaatagcgaaagtagtcatcaaaatctgtgaacatgagatgaatgaaatcgaagtatgtccagaatgttatctagctgcttgccaaaaacgagataactggttttgtgagccttgtagcaatccacatcctttggtctgggccaaactgaaggggtttccattctggcctgcaaaagctctaagggataaagacgggcaggtcgatgcccgattctttggacaacatgacagggcctgggttccaataaataattgctacctcatgtctaaagaaattcctttttctgtgaaaaagactaagagcatcttcaacagtgccatgcaagagatggaggtttacgtggagaacatccgcaggaagtttggggtttttaattactctccatttaggacaccctacacacccaacagccagtatcaaatgctgctcgatcccaccaaccccagcgccggcactgccaagatagacaagcaggagaaggtcaagctcaactttgacatgacggcatcccccaagatcctgatgagcaagcctgtgctgagtgggggcacaggccgccggatttccttgtcggatatgccgcgctcccccatgagcacaaactcttctgtgcacacgggctccgacgtggagcaggatgctgagaagaaggccacgtcgagccacttcagtgcgagcgaggagtccatggacttcctggataagagcacagcttcaccagcctccaccaagacgggacaagcagggagtttatccggcagcccaaagcccttctctcctcaactgtcagctcctatcacgacgaaaacggacaaaacctccaccaccggcagcatcctgaatcttaacctggatcgaagcaaagctgagatggatttgaaggagctgagcgagtcggtccagcaacagtccacccctgttcctctcatctctcccaagcgccagattcgtagcaggttccagctgaatcttgacaagaccatagagagttgcaaagcacaattaggcataaatgaaatctcggaagatgtctatacggccgtagagcacagcgattcggaggattctgagaagtcagatagtagcgatagtgagtatatcagtgatgatgagcagaagtctaagaacgagccagaagacacagaggacaaagaaggttgtcagatggacaaagagccatctgctgttaaaaaaaagcccaagcctacaaacccagtggagattaaagaggagctgaaaagcacgtcaccagccagcgagaaggcagaccctggagcagtcaaggacaaggccagccctgagcctgagaaggacttttccgaaaaggcaaaaccttcacctcaccccataaaggataaactgaagggaaaagatgagacggattccccaacagtccatttgggcctggactctgattcagagagcgaacttgtcatagatttaggagaagaccattctgggcgggagggtcgaaaaaataagaaggaacccaaagaaccatctcccaaacaggatgttgtaggtaaaactccaccatccacgacggtgggcagccattctcccccggaaacaccggtgctcacccgctcttccgcccaaacttccgcggctggcgccacagccaccaccagcacgtcctccacggtcaccgtcacggccccggcccccgccgccacaggaagcccagtgaaaaagcagaggccgcttttaccgaaggagactgccccggccgtgcagcgggtcgtgtggaactcatcaactgtccagcagaaggagatcacacagagcccatccacgtccaccatcaccctggtgaccagcacacagtcatcgcccctggtcaccagctcggggtccatgagcacccttgtgtcctcagtcaacgctgacctgcccatcgccactgcctcagctgatgtcgccgctgatattgccaagtacactagcaaaatgatggatgcaataaaaggaacaatgacagaaatatacaacgatctttctaaaaacactactggaagcacaatagctgagattcgcaggctgaggatcgagatagagaagctccagtggctgcaccagcaagagctctccgaaatgaaacacaacttagagctgaccatggcggagatgcggcagagcctggagcaggagcgggaccggctcatcgccgaggtgaagaagcagctggagttggagaagcagcaggcggtggatgagaccaagaagaagcagtggtgcgccaactgcaagaaggaggccatcttttactgctgttggaacaccagctactgtgactacccctgccagcaagcccactggcctgagcacatgaagtcctgcacccagtcagctactgctcctcagcaggaagcggatgctgaggtgaacacagaaacactaaataagtcctcccaggggagctcctcgagcacacaatcagcaccttcagaaacggccagcgcctccaaagagaaggagacgtcagctgagaaaagcaaggagagtggctcgacccttgacctttctggctccagagagacgccctcctccattctcttaggctccaaccaaggctctgttagcaaaaggtgtgacaagcaacctgcctatgccccaaccaccacagaccaccagccgcaccccaactaccccgcccagaagtaccattcccggagtaataaatccagttggagcagcagtgatgagaagaggggatcgacacgttccgatcacaacaccagtaccagcacgaagagcctcctcccgaaagagtctcggctggacaccttctgggactag

SEQ ID NO: 21 - DNA, 942 bp, Homo sapiens
atgtcccagagcaggcaccgcgccgaggccccgccgctggagcgcgaggacagtgggaccttcagtttggggaagatgataacagctaagccagggaaaacaccgattcaggtattacacgaatacggcatgaagaccaagaacatcccagtttatgaatgtgaaagatctgatgtgcaaatacacgtgcccactttcaccttcagagtaaccgttggtgacataacctgcacaggtgaaggtacaagtaagaagctggcgaaacatagagctgcagaggctgccataaacattttgaaagccaatgcaagtatttgctttgcagttcctgaccccttaatgcctgacccttccaagcaaccaaagaaccagcttaatcctattggttcattacaggaattggctattcatcatggctggagacttcctgaatataccctttcccaggagggaggacctgctcataagagagaatatactacaatttgcaggctagagtcatttatggaaactggaaagggggcatcaaaaaagcaagccaaaaggaatgctgctgagaaatttcttgccaaatttagtaatatttctccagagaaccacatttctttaacaaatgtagtaggacattctttaggatgtacttggcattccttgaggaattctcctggtgaaaagatcaacttactgaaaagaagcctccttagtattccaaatacagattacatccagctgcttagtgaaattgccaaggaacaaggttttaatataacatatttggatatagatgaactgagcgccaatggacaatatcaatgtcttgctgaactgtccaccagccccatcacagtctgtcatggctccggtatctcctgtggcaatgcacaaagtgatgcagctcacaatgctttgcagtatttaaagataatagcagaaagaaagtaa

SEQ ID NO: 22 - DNA, 765 bp, Homo sapiens
atggcctcgttgctgaaggtggatcaggaagtgaagctcaaggttgattctttcagggagcggatcacaagtgaggcagaagacttggtggcaaattttttcccaaagaagttattagaacttgatagttttctgaaggaaccaatcttaaacatccatgacctaactcagatccactctgacatgaatctcccagtccctgaccccattcttctcaccaatagccatgatggactggatggtcccacttataagaagcgaaggttggatgagtgtgaagaagccttccaaggaaccaaggtgtttgtgatgcccaatgggatgctgaaaagcaaccagcagctggtggacattattgagaaagtgaaacctgagatccggctgttgattgagaaatgtaacacggtcaaaatgtgggtacagctcctgattcccaggatagaagatggaaacaactttggggtgtccattcaggaggaaacagttgcagagctaagaactgttgagagtgaagctgcatcttatctggaccagatttctagatattatattacaagagccaaattggtttctaaaatagctaaatatccccatgtggaggactatcgccgcaccgtgacagagattgatgagaaagaatatatcagccttcggctcatcatatcagagctgaggaatcaatatgtcactctacatgacatgatcctgaaaaatatcgagaagatcaaacggccccggagcagcaatgcagagactctgtactga

SEQ ID NO: 23 - DNA, 2271 bp, Homo sapiens
atggcgtcggccccgccggcctcgcccccgggctcggagccgccggggcccgacccggagccgggcgggccggacgggccgggggcggcgcaactggctccgggccctgcggagctacgcctcggagcgcccgtcggcggccccgacccgcagtccccgggcctggatgagcctgcgcccggggccgctgcagatggcggggcgcgttggagcgccgggccggccccggggctggagggaggcccgcgagaccccgggccgtccgccccgccgccgcgctccggcccgcgggggcagcttgcgagccccgacgccccgggcccagggccgcgctccgaagcgccgcttccagaactcgacccgttgttctcctggactgaggagcccgaggagtgtggccccgcgagctgcccggagagcgcgcctttccgcttgcaggggtccagcagcagccaccgagcgcggggcgaggtcgacgtcttctctcccttccccgcgcccacggcgggcgagctggcgctggagcaaggtcccgggtccccgccgcagccctcggacctcagccagacccacccccttccgagcgagcccgtggggagtcaggaggacggcccccgcctccgagccgtgttcgatgccctggacggggatggggacggtttcgtccgcatcgaggacttcatccagtttgctacggtctacggggcagagcaggtgaaggacttaactaagtacttggatcccagtgggctcggcgtgatcagctttgaagacttctaccaagggatcacagccatcagaaacggagatcctgatggccagtgctacggtggtgtcgcttctgcccaagatgaggagcccctggcctgcccggacgagttcgatgacttcgtcacctatgaggccaacgaggtgacggacagcgcgtacatgggctccgagagcacctacagtgagtgtgagaccttcacggacgaggacaccagcaccctggtgcaccctgagctgcaacctgaaggggacgcagacagtgccggcggctcggccgtgccctctgagtgcctggacgccatggaggagcccgaccatggtgccctgctgctgctcccaggcaggcctcacccccatggccagtctgtcatcacggtgatcgggggcgaggagcactttgaggactacggtgaaggcagtgaggcggagctgtccccagagaccctatgcaacgggcagctgggctgcagtgaccccgctttcctcacgcccagtccgacaaagcggctctccagcaagaaggtggcaaggtacctgcaccagtcaggggccctgaccatggaggccctggaggacccttcccccgagctcatggagggcccagaggaggacattgctgacaaggttgtcttcctggaaaggcgtgtgctggagctggaaaaggacacggcagccaccggtgagcaacacagccgcctgaggcaggagaacctgcagctggtgcacagagcaaacgccctggaggagcagctgaaggagcaggagctgagagcctgcgagatggtcctggaagagacccggcgtcagaaggagctcctgtgcaagatggagagggagaagagcattgagatcgagaacctgcagaccaggctacagcaactggacgaggagaacagtgaactccggtcctgcacgccctgtctgaaggccaacattgagcgtctggaggaggagaagcagaagctgttggatgagatagagtcgctgacgctgcggctcagtgaagagcaggagaacaagaggagaatgggggacaggctgagtcacgagaggcaccagttccagagggacaaggaggccacccaggagctgatcgaggacctccgaaagcagctggagcacctgcagctcctcaagctggaggccgagcagcggcggggccgcagcagcagcatgggcctgcaggagtaccacagccgcgcccgggagagcgagctggagcaggaggtccgcaggctgaagcaggacaaccgcaacctgaaggagcagaacgaggagctgaacgggcagatcattaccctcagcatccagggcgccaagagcctcttctccacagccttctctgagtccctggctgcagagatcagctccgtctcccgagatgagctcatggaggcgattcagaagcaggaggagatcaacttccgcctgcaggactacatcgacaggatcatcgtggccatcatggagaccaacccgtccatcctggaggtcaagtag

SEQ ID NO: 24 - DNA, 1968 bp, Homo sapiens
atgactgagtgcttcctgccccccaccagcagccccagtgaacaccgcagggtggagcatggcagcgggcttacccggacccccagctctgaagagatcagccctactaagtttcctggattgtaccgcactggcgagccctcacctccccatgacatcctccatgagcctcctgatgtagtgtctgatgatgagaaagatcatgggaagaaaaaagggaaatttaagaaaaaggaaaagaggactgaaggctatgcagcctttcaggaagatagctctggagatgaggcagaaagtccttctaaaatgaagaggtccaagggaatccatgttttcaagaagcccagcttttctaaaaagaaggaaaaggattttaaaataaaagagaaacccaaagaagaaaagcataaagaagaaaagcacaaagaagaaaaacataaagagaagaagtcaaaagacttgacagcagctgatgttgttaaacagtggaaggaaaagaagaaaaagaaaaagccaattcaggagccagaggtgcctcagattgatgttccaaatctcaaacccatttttggaattcctttggctgatgcagtagagaggaccatgatgtatgatggcattcggctgccagccgttttccgtgaatgtatagattacgtagagaagtatggcatgaagtgtgaaggcatctacagagtatcaggaattaaatcaaaggtggatgagctaaaagcagcctatgaccgggaggagtctacaaacttggaagactatgagcctaacactgtagccagtttgctgaagcagtatttgcgagaccttccagagaatttgcttaccaaagagcttatgcccagatttgaagaggcttgtgggaggaccacggagactgagaaagtgcaggaattccagcgtttactcaaagaactgccagaatgtaactatcttctgatttcttggctcattgtgcacatggaccatgtcattgcaaaggaactggaaacaaaaatgaatatacagaacatttctatagtgctcagcccaactgtgcagatcagcaatcgagtcctgtatgtgtttttcacacatgtgcaagaactctttggaaatgtggtactaaagcaagtgatgaaacctctgcgatggtctaacatggccacgatgcccacgctgccagagacccaggcgggcatcaaggaggagatcaggagacaggagtttcttttgaattgtttacatcgagatctgcagggtgggataaaggatttgtctaaagaagaaagattatgggaagtacaaagaattttgacagccctcaaaagaaaactgagagaagctaaaagacaggagtgtgaaaccaagattgcacaagagatagccagtctttcaaaagaggatgtttccaaagaagagatgaatgaaaatgaagaagttataaatattctccttgctcaggagaatgagatcctgactgaacaggaggagctcctggccatggagcagtttctgcgccggcagattgcctcagaaaaagaagagattgaacgcctcagagctgagattgctgaaattcagagtcgccagcagcacggccgaagtgagactgaggagtactcctccgagagcgagagcgagagtgaggatgaggaggagctgcagatcattctggaagacttacagagacagaacgaagagctggaaataaagaacaatcatttgaatcaagcaattcatgaggagcgcgaggccatcatcgagctgcgcgtgcagctgcggctgctccagatgcagcgagccaaggccgagcagcaggcgcaggaggacgaggagcctgagtggcgcgggggtgccgtccagccgcccagagacggcgtccttgagccaaaagcagctaaagagcagccaaaggcaggcaaggagccggcaaagccatcgcccagcagggataggaaggagacgtccatctga

SEQ ID NO: 25 - DNA, 651 bp, Homo sapiens
atggctgcgcagggagagccccaggtccagttcaaacttgtattggttggtgatggtggtactggaaaaacgaccttcgtgaaacgtcatttgactggtgaatttgagaagaagtatgtagccaccttgggtgttgaggttcatcccctagtgttccacaccaacagaggacctattaagttcaatgtatgggacacagccggccaggagaaattcggtggactgagagatggctattatatccaagcccagtgtgccatcataatgtttgatgtaacatcgagagttacttacaagaatgtgcctaactggcatagagatctggtacgagtgtgtgaaaacatccccattgtgttgtgtggcaacaaagtggatattaaggacaggaaagtgaaggcgaaatccattgtcttccaccgaaagaagaatcttcagtactacgacatttctgccaaaagtaactacaactttgaaaagcccttcctctggcttgctaggaagctcattggagaccctaacttggaatttgttgccatgcctgctctcgccccaccagaagttgtcatggacccagctttggcagcacagtatgagcacgacttagaggttgctcagacaactgctctcccggatgaggatgatgacctgtga

SEQ ID NO: 26 - DNA, 1389 bp, Homo sapiens
atggccagcaacagcagctcctgcccgacacctgggggcgggcacctcaatgggtacccggtgcctccctacgccttcttcttcccccctatgctgggtggactctccccgccaggcgctctgaccactctccagcaccagcttccagttagtggatatagcacaccatccccagccaccattgagacccagagcagcagttctgaagagatagtgcccagccctccctcgccaccccctctaccccgcatctacaagccttgctttgtctgtcaggacaagtcctcaggctaccactatggggtcagcgcctgtgagggctgcaagggcttcttccgccgcagcatccagaagaacatggtgtacacgtgtcaccgggacaagaactgcatcatcaacaaggtgacccggaaccgctgccagtactgccgactgcagaagtgctttgaagtgggcatgtccaaggagtctgtgagaaacgaccgaaacaagaagaagaaggaggtgcccaagcccgagtgctctgagagctacacgctgacgccggaggtgggggagctcattgagaaggtgcgcaaagcgcaccaggaaaccttccctgccctctgccagctgggcaaatacactacgaacaacagctcagaacaacgtgtctctctggacattgacctctgggacaagttcagtgaactctccaccaagtgcatcattaagactgtggagttcgccaagcagctgcccggcttcaccaccctcaccatcgccgaccagatcaccctcctcaaggctgcctgcctggacatcctgatcctgcggatctgcacgcggtacacgcccgagcaggacaccatgaccttctcggacgggctgaccctgaaccggacccagatgcacaacgctggcttcggccccctcaccgacctggtctttgccttcgccaaccagctgctgcccctggagatggatgatgcggagacggggctgctcagcgccatctgcctcatctgcggagaccgccaggacctggagcagccggaccgggtggacatgctgcaggagccgctgctggaggcgctaaaggtctacgtgcggaagcggaggcccagccgcccccacatgttccccaagatgctaatgaagattactgacctgcgaagcatcagcgccaagggggctgagcgggtgatcacgctgaagatggagatcccgggctccatgccgcctctcatccaggaaatgttggagaactcagagggcctggacactctgagcggacagccggggggtggggggcgggacgggggtggcctggcccccccgccaggcagctgtagccccagcctcagccccagctccaacagaagcagcccggccacccactccccgtga

SEQ ID NO: 27 - DNA, 1212 bp, Homo sapiens
atgggcaaagtgtggaaacagcagatgtaccctcagtacgccacctactattacccccagtatctgcaagccaagcagtctctggtcccagcccaccccatggcccctcccagtcccagcaccaccagcagtaataacaacagtagcagcagtagcaactcaggatgggatcagctcagcaaaacgaacctctatatccgaggactgcctccccacaccaccgaccaggacctggtgaagctctgtcaaccatatgggaaaatagtctccacaaaggcaattttggataagacaacaaacaaatgcaaaggttatggttttgtcgactttgacagccctgcagcagctcaaaaagctgtgtctgccctgaaggccagtggggttcaagctcaaatggcaaagcaacaggaacaagatcctaccaacctctacatttctaatttgccactctccatggatgagcaagaactagaaaatatgctcaaaccatttggacaagttatttctacaaggatactacgtgattccagtggtacaagtcgtggtgttggctttgctaggatggaatcaacagaaaaatgtgaagctgttattggtcattttaatggaaaatttattaagacaccaccaggagtttctgcccccacagaacctttattgtgtaagtttgctgatggaggacagaaaaagagacagaacccaaacaaatacatccctaatggaagaccatggcatagagaaggagaggctggaatgacacttacttacgacccaactacagctgctatacagaacggattttatccttcaccatacagtattgctacaaaccgaatgatcactcaaacttctattacaccctatattgcatctcctgtatctgcctaccaggtgcaaagtccttcgtggatgcaacctcaaccatatattctacagcaccctggtgccgtgttaactccctcaatggagcacaccatgtcactacagcccgcatcaatgatcagccctctggcccagcagatgagtcatctgtcactaggcagcaccggaacatacatgcctgcaacgtcagctatgcaaggagcctacttgccacagtatgcacatatgcagacgacagcggttcctgttgaggaggcaagtggtcaacagcaggtggctgtcgagacgtctaatgaccattctccatatacctttcaacctaataagtaa

SEQ ID NO: 28 - DNA, 1143 bp, Homo sapiens
atgttggtgataccccccggactgagcgaggaagaggaggctctgcagaagaaattcaacaagctcaagaaaaagaaaaaggcattgctggctctgaagaagcaaagtagcagcagcacaaccagccaaggtggtgtcaaacgctcactatcagagcagcctgtcatggacacagccacagcaacagagcaggcaaagcagctggtgaagtcaggagccatcagtgccatcaaggctgagaccaagaactcaggcttcaagcgttctcgaacccttgaggggaagttaaaggaccccgagaagggaccagtccccactttccagccgttccagaggagcatatctgctgatgatgacctgcaagagtcatccagacgtccccagaggaaatctctgtatgagagctttgtgtcttctagtgatcgacttcgagaactaggaccagatggagaagaggcagagggcccaggggctggtgatggtccccctcgaagctttgactggggctatgaagaacgcagtggtgcccactcctcagcctcccctccccgaagccgcagccgggaccgcagccatgagaggaaccgggacagagaccgagatcgggagcgggatcgagaccgggatcgagacagagacagagagcgggacagggatcgggatcgggatcgagatcgagaccgggaacgggacagggatcgggagcgggatcgagaccgagaccgagagggtcctttccgcaggtcggattcattccctgaacggcgagcccctaggaaagggaatactctctatgtatatggagaagacatgacacccacccttctccgtggggccttctctccttttggaaacatcattgacctctccatggacccacccagaaactgtgccttcgtcacctatgaaaagatggagtcagcagatcaggccgttgctgagctcaacgggacccaggtggagtctgtacagctcaaagtcaacatagcccgaaaacagcccatgctggatgccgctactggcaagtctgtctggggctccctcgctgtccagaacagccctaagggttgccaccgggacaagaggacccagattgtctacagtgatgacgtctacaaggaaaaccttgtggatggcttctag

SEQ ID NO: 29 - DNA, 1875 bp, Homo sapiens
atggaaaactcagattccaatgacaaaggaagtggtgatcagtctgcagcacagcgcagaagtcagatggaccgattggatcgagaagaagctttctatcaatttgtaaataacctgagtgaagaagattataggcttatgagagataacaatttgctaggcaccccaggtgaaagtactgaggaagagttgctgagacgactacagcaaattaaagaaggcccaccaccgcaaaactcagatgaaaatagaggaggagactcttcagatgatgtgtctaatggtgactctataatagactggcttaactctgtcagacaaactggaaatacaacaagaagtgggcaaagaggaaaccaatcttggagagcagtgagtcggactaatccaaacagtggtgatttcagattcagtttagagataaatgttaaccgtaataatgggagccaaaattcagagaatgaaaatgagccatctgcaagacgttctagtggagaaaatgtggaaaacaacagccaaaggcaagtggaaaacccacgatctgaatcaacatctgcaaggccatctagatcagaacgaaattcaactgaagcgttaacagaggtcccacctaccagaggtcagaggagggcaagaagcaggagcccagaccatcggagaaccagagcaagagctgaaagaagtaggtcacctctgcatccaatgagtgaaattccacgaagatctcatcatagtatctcatctcagacttttgaacatcctttggtaaatgagacggagggaagttctagaacccggcaccatgtgacattgaggcagcaaatatctgggcctgagttgctaagtagaggtctttttgcagcttctggaacaagaaatgcttctcaaggagcaggttcttcagacacagctgccagtggtgaatctacaggatcaggacagagacctccaaccatagtccttgatcttcaagtaagaagagttcgtcctggagaatatcggcagagagatagcatagccagcagaactcggtctaggtctcagacaccaaacaacactgtcacctatgaaagtgaacgaggaggttttaggcgtacattttcacgttctgagcgggcaggtgtgagaacctatgtcagtaccatcagaattcccattcgtagaatcttaaatactggtttaagtgagactacatctgttgcaattcagaccatgttaaggcagataatgacaggttttggtgagttaagctattttatgtacagtgatagcgactcagagcctactggctcagtctcaaatcgaaatatggaaagggcagagtcacggagtggaagaggaggttctggtggtggtagtagttctggttccagttcgagttccagttccagttcgagttccagttccagttcaagttccagttccagtcctagttccagttccggtggtgaaagttcagaaactagctcagatttatttgaaggcagtaatgaaggaagctcatcatcaggctcatcaggtgccaggcgagagggtcgacatagggccccagtcacatttgatgaaagtggctctttgcccttccttagcctggctcagtttttcctcttaaatgaggatgatgatgaccaacctagaggactcaccaaagaacagattgacaacttggcaatgagaagttttggtgaaaatgatgcattaaaaacctgtagtgtttgcattacagaatatacagaaggcaacaaacttcgtaaactaccttgttcccatgagtaccatgtccactgcatcgatcgctggttatctgagaattctacctgtcctatttgtcgcagagcagtcttagcttctggtaacagagaaagtgttgtgtaa

SEQ ID NO: 30 - DNA, 348 bp, Homo sapiens
atggtggccgcaaagaagacgaaaaagtcgctggagtcgatcaactctaggctccaactcgttatgaaaagtgggaagtacgtcctggggtacaagcagactctgaagatgatcagacaaggcaaagcgaaattggtcattctcgctaacaactgcccagctttgaggaaatctgaaatagagtactatgctatgttggctaaaactggtgtccatcactacagtggcaataatattgaactgggcacagcatgcggaaaatactacagagtgtgcacactggctatcattgatccaggtgactctgacatcattagaagcatgccagaacagactggtgaaaagtaa

SEQ ID NO: 31 - DNA, 378 bp, Homo sapiens
atggctcccgcaaagaagggtggcgagaagaaaaagggccgttctgccatcaacgaagtggtaacccgagaatacaccatcaacattcacaagcgcatccatggagtgggcttcaagaagcgtgcacctcgggcactcaaagagattcggaaatttgccatgaaggagatgggaactccagatgtgcgcattgacaccaggctcaacaaagctgtctgggccaaaggaataaggaatgtgccataccgaatccgtgtgcggctgtccagaaaacgtaatgaggatgaagattcaccaaataagctatatactttggttacctatgtacctgttaccactttcaaaaatctacagacagtcaatgtggatgagaactaa

SEQ ID NO: 32 - DNA, 1803 bp, Homo sapiens
atgatggaggagcgtgccaacctgatgcacatgatgaaactcagcatcaaggtgttgctccagtcggctctgagcctgggccgcagcctggatgcggaccatgcccccttgcagcagttctttgtagtgatggagcactgcctcaaacatgggctgaaagttaagaagagttttattggccaaaataaatcattctttggtcctttggagctggtggagaaactttgtccagaagcatcagatatagcgactagtgtcagaaatcttccagaattaaagacagctgtgggaagaggccgagcgtggctttatcttgcactcatgcaaaagaaactggcagattatctgaaagtgcttatagacaataaacatctcttaagcgagttctatgagcctgaggctttaatgatggaggaagaagggatggtgattgttggtctgctggtgggactcaatgttctcgatgccaatctctgcttgaaaggagaagacttggattctcaggttggagtaatagatttttccctctaccttaaggatgtgcaggatcttgatggtggcaaggagcatgaaagaattactgatgtccttgatcaaaaaaattatgtggaagaacttaaccggcacttgagctgcacagttggggatcttcaaaccaagatagatggcttggaaaagactaactcaaagcttcaagaagagctttcagctgcaacagaccgaatttgctcacttcaagaagaacagcagcagttaagagaacaaaatgaattaattcgagaaagaagtgaaaagagtgtagagataacaaaacaggataccaaagttgagctggagacttacaagcaaactcggcaaggtctggatgaaatgtacagtgatgtgtggaagcagctaaaagaggagaagaaagtccggttggaactggaaaaagaactggagttacaaattggaatgaaaaccgaaatggaaattgcaatgaagttactggaaaaggacacccacgagaagcaggacacactagttgccctccgccagcagctggaagaagtcaaagcgattaatttacagatgtttcacaaagctcagaatgcagagagcagtttgcagcagaagaatgaagccatcacatcctttgaaggaaaaaccaaccaagttatgtccagcatgaaacaaatggaagaaaggttgcagcactcggagcgggcgaggcagggagctgaggagcggagccacaagctgcagcaggagctgggcgggaggatcggcgccctgcagctgcagctctcccagctgcacgagcaatgctcaagcctggagaaagaattgaaatcagaaaaagagcaaagacaggctcttcagcgcgaattacagcacgagaaagacacttcctctctactcaggatggagctgcaacaagtggaaggactgaaaaaggagttgcgggagcttcaggacgagaaggcagagctgcagaagatctgcgaggagcaggaacaagccctccaggaaatgggcctgcacctcagccagtccaagctgaagatggaagatataaaagaagtgaaccaggcactgaagggccacgcctggctgaaagatgacgaagcgacacactgtaggcagtgtgagaaggagttctccatttcccggagaaagcaccactgccggaactgtggccacatcttctgcaacacctgctccagcaacgagctggccctgccctcctaccccaagccggtgcgagtgtgcgacagctgccacaccctgctcctgcagcgctgctcctccacggcctcctga

SEQ ID NO: 33 - DNA, 849 bp, Homo sapiens
atggcgatgagcagcggcggcagtggtggcggcgtcccggagcaggaggattccgtgctgttccggcgcggcacaggccagagcgatgattctgacatttgggatgatacagcactgataaaagcatatgataaagctgtggcttcatttaagcatgctctaaagaatggtgacatttgtgaaacttcgggtaaaccaaaaaccacacctaaaagaaaacctgctaagaagaataaaagccaaaagaagaatactgcagcttccttacaacagtggaaagttggggacaaatgttctgccatttggtcagaagacggttgcatttacccagctaccattgcttcaattgattttaagagagaaacctgtgttgtggtttacactggatatggaaatagagaggagcaaaatctgtccgatctactttccccaatctgtgaagtagctaataatatagaacagaatgctcaagagaatgaaaatgaaagccaagtttcaacagatgaaagtgagaactccaggtctcctggaaataaatcagataacatcaagcccaaatctgctccatggaactcttttctccctccaccaccccccatgccagggccaagactgggaccaggaaagccaggtctaaaattcaatggcccaccaccgccaccgccaccaccaccaccccacttactatcatgctggctgcctccatttccttctggaccaccaataattcccccaccacctcccatatgtccagattctcttgatgatgctgatgctttgggaagtatgttaatttcatggtacatgagtggctatcatactggctattatatggaaatgctggcatag

SEQ ID NO: 34 - DNA, 2058 bp, Homo sapiens
atggagcttttgcggactatcacctaccagccagccgccagcaccaaaatgtgcgagcaggcgctgggcaagggttgcggagcagactcgaagaagaagcggccgccgcagccccccgaggaatcgcagccacctcagtcccaggcgcaagtgcccccggcggcccctcaccaccatcaccaccattcgcactcggggccggagatctcgcggattatcgtcgaccccacgactgggaagcgctactgccggggcaaagtgctgggaaagggtggctttgcaaaatgttacgagatgacagatttgacaaataacaaagtctacgccgcaaaaattattcctcacagcagagtagctaaacctcatcaaagggaaaagattgacaaagaaatagagcttcacagaattcttcatcataagcatgtagtgcagttttaccactacttcgaggacaaagaaaacatttacattctcttggaatactgcagtagaaggtcaatggctcatattttgaaagcaagaaaggtgttgacagagccagaagttcgatactacctcaggcagattgtgtctggactgaaataccttcatgaacaagaaatcttgcacagagatctcaaactagggaacttttttattaatgaagccatggaactaaaagttggggacttcggtctggcagccaggctagaacccttggaacacagaaggagaacgatatgtggtaccccaaattatctctctcctgaagtcctcaacaaacaaggacatggctgtgaatcagacatttgggccctgggctgtgtaatgtatacaatgttactagggaggcccccatttgaaactacaaatctcaaagaaacttataggtgcataagggaagcaaggtatacaatgccgtcctcattgctggctcctgccaagcacttaattgctagtatgttgtccaaaaacccagaggatcgtcccagtttggatgacatcattcgacatgacttttttttgcagggcttcactccggacagactgtcttctagctgttgtcatacagttccagatttccacttatcaagcccagctaagaatttctttaagaaagcagctgctgctctttttggtggcaaaaaagacaaagcaagatatattgacacacataatagagtgtctaaagaagatgaagacatctacaagcttaggcatgatttgaaaaagacttcaataactcagcaacccagcaaacacaggacagatgaggagctccagccacctaccaccacagttgccaggtctggaacacccgcagtagaaaacaagcagcagattggggatgctattcggatgatagtcagagggactcttggcagctgtagcagcagcagtgaatgccttgaagacagtaccatgggaagtgttgcagacacagtggcaagggttcttcggggatgtctggaaaacatgccggaagctgattgcattcccaaagagcagctgagcacatcatttcagtgggtcaccaaatgggttgattactctaacaaatatggctttgggtaccagctctcagaccacaccgtcggtgtccttttcaacaatggtgctcacatgagcctccttccagacaaaaaaacagttcactattacgcagagcttggccaatgctcagttttcccagcaacagatgctcctgagcaatttattagtcaagtgacggtgctgaaatacttttctcattacatggaggagaacctcatggatggtggagatctgcctagtgttactgatattcgaagacctcggctctacctccttcagtggctaaaatctgataaggccctaatgatgctctttaatgatggcacctttcaggtgaatttctaccatgatcatacaaaaatcatcatctgtagccaaaatgaagaataccttctcacctacatcaatgaggataggatatctacaactttcaggctgacaactctgctgatgtctggctgttcatcagaattaaaaaatcgaatggaatatgccctgaacatgctcttacaaagatgtaactga

SEQ ID NO: 35 - DNA, 1968 bp, Homo sapiens
atggagcggaaagtgcttgcgctccaggcccgaaagaaaaggaccaaggccaagaaggacaaagcccaaaggaaatctgaaactcagcaccgaggctctgctccccactctgagagtgatctaccagagcaggaagaggagattctgggatctgatgatgatgagcaagaagatcctaatgattattgtaaaggaggttatcatcttgtgaaaattggagatctattcaatgggagataccatgtgatccgaaagttaggctggggacacttttcaacagtatggttatcatgggatattcaggggaagaaatttgtggcaatgaaagtagttaaaagtgctgaacattacactgaaacagcactagatgaaatccggttgctgaagtcagttcgcaattcagaccctaatgatccaaatagagaaatggttgttcaactactagatgactttaaaatatcaggagttaatggaacacatatctgcatggtatttgaagttttggggcatcatctgctcaagtggatcatcaaatccaattatcaggggcttccactgccttgtgtcaaaaaaattattcagcaagtgttacagggtcttgattatttacataccaagtgccgtatcacccacactgacattaaaccagagaacatcttattgtcagtgaatgagcagtacattcggaggctggctgcagaagcaacagaatggcagcgatctggagctcctccgccttccggatctgcagtcagtactgctccccagcctaaaccagctgacaaaatgtcaaagaataagaagaagaaattgaagaagaagcagaagcgccaggcagaattactagagaagcgaatgcaggaaattgaggaaatggagaaagagtcgggccctgggcaaaaaagaccaaacaagcaagaagaatcagagagtcctgttgaaagacccttgaaagagaacccacctaataaaatgacccaagaaaaacttgaagagtcaagtaccattggccaggatcaaacgcttatggaacgtgatacagagggtggtgcagcagaaattaattgcaatggagtgattgaagtcattaattatactcagaacagtaataatgaaacattgagacataaagaggatctacataatgctaatgactgtgatgtccaaaatttgaatcaggaatctagtttcctaagctcccaaaatggagacagcagcacatctcaagaaacagactcttgtacacctataacatctgaggtgtcagacaccatggtgtgccagtcttcctcaactgtaggtcagtcattcagtgaacaacacattagccaacttcaagaaagcattcgggcagagataccctgtgaagatgaacaagagcaagaacataacggaccactggacaacaaaggaaaatccacggctggaaattttcttgttaatccccttgagccaaaaaatgcagaaaagctcaaggtgaagattgctgaccttggaaatgcttgttgggtgcacaaacatttcactgaagatattcaaacaaggcaatatcgttccttggaagttctaatcggatctggctataatacccctgctgacatttggagcacggcatgcatggcctttgaactggccacaggtgactatttgtttgaacctcattcaggggaagagtacactcgagatgaagatcacattgcattgatcatagaacttctggggaaggtgcctcgcaagctcattgtggcaggaaaatattccaaggaatttttcaccaaaaaaggtgacctgaaacatatcacgaagctgaaaccttggggcctttttgaggttctagtggagaagtatgagtggtcgcaggaagaggcagctggcttcacagatttcttactgcccatgttggagctgatccctgagaagagagccactgccgccgagtgtctccggcacccttggcttaactcctaa

SEQ ID NO: 36 - DNA, 1617 bp, Homo sapiens
atggaggaatctgtaaaccaaatgcagccactgaatgagaagcagatagccaattctcaggatggatatgtatggcaagtcactgacatgaatcgactacaccggttcttatgtttcggttctgaaggtgggacttattatatcaaagaacagaagttgggccttgaaaatgctgaagctttaattagattgattgaagatggcagaggatgtgaagtgatacaagaaataaagtcatttagtcaagaaggcagaaccacaaagcaagagcctatgctctttgcacttgccatttgttcccagtgctccgacataagcacaaaacaagcagcatttaaagctgtttctgaagtttgtcgcattcctacccatctctttacttttatccagtttaagaaagatctgaaggaaagcatgaaatgtggcatgtggggtcgtgccctccggaaggctatagcggactggtacaatgagaaaggtggcatggcccttgctctggcagttacaaaatataaacagagaaatggctggtctcacaaagatctattaagattgtcacatcttaaaccttccagtgaaggacttgcaattgtgaccaaatatattacaaagggctggaaagaagttcatgaattgtataaagaaaaagcactctctgtggagactgaaaaattattaaagtatctggaggctgtagagaaagtgaagcgcacaagagatgagctagaagtcattcatctaatagaagaacatagattagttagagaacatcttttaacaaatcacttaaagtctaaagaggtatggaaggctttgttacaagaaatgccgcttactgcattactaaggaatctaggaaagatgactgctaattcagtacttgaaccaggaaattcagaagtatctttagtatgtgaaaaactgtgtaatgaaaaactattaaaaaaggctcgtatacatccatttcatattttgatcgcattagaaacttacaagacaggtcatggtctcagagggaaactgaagtggcgccctgatgaagaaattttgaaagcattggatgctgctttttataaaacatttaagacagttgaaccaactggaaaacgtttcttactagctgttgatgtcagtgcttctatgaaccaaagagttttgggtagtatactcaacgctagtacagttgctgcagcaatgtgcatggttgtcacacgaacagaaaaagattcttatgtagttgctttttccgatgaaatggtaccatgtccagtgactacagatatgaccttacaacaggttttaatggctatgagtcagatcccagcaggtggaactgattgctctcttccaatgatctgggctcagaagacaaacacacctgctgatgtcttcattgtattcactgataatgagacctttgctggaggtgtccatcctgctattgctctgagggagtatcgaaagaaaatggatattccagctaaattgattgtttgtggaatgacatcaaatggtttcaccattgcagacccagatgatagaggcatgttggatatgtgcggctttgatactggagctctggatgtaattcgaaatttcacattagatatgatttaa

SEQ ID NO: 37 - DNA, 360 bp, Homo sapiens
atgacccagcagggcgcggcgctgcagaactacaacaacgagctggtcaagtgcatagaggagctgtgccagaagcgggaggagctgtgccggcagatccaggaggaggaggacgagaagcagcggctgcagaatgaggtgaggcagctgacagagaagctggcccgcgtcaacgagaacctggcacgcaagattgcctctcgcaacgagttcgaccggaccatcgcggagacggaggccgcctacctcaagatcctggagagctcccagactttgctcagcgttctcaagagggaagctgggaacctgaccaaggctacagccccagaccagaaaagtagcggcggcagggacagctga

SEQ ID NO: 38 - DNA, 1734 bp, Homo sapiens
atgtctcaagttcaagtgcaagttcagaacccatctgctgctctctcagggagccaaatactgaacaagaaccagtctcttctctcacagcctttgatgagtattccttctactactagctctctgccctctgaaaatgcaggtagacccattcaaaactctgctttaccctctgcatctattacatccaccagtgcagctgcagaaagcataacccctactgtagaactaaatgcactgtgcatgaaacttggaaaaaaaccaatgtataagcctgttgacccttactctcggatgcagtccacctataactacaacatgagaggaggtgcttatcccccgaggtacttttacccatttccagttccacctttactttatcaagtggaactttctgtgggaggacagcaatttaatggcaaaggaaagacaagacaggctgcgaaacacgatgctgctgccaaagcgttgaggatcctgcagaatgagcccctgccagagaggctggaggtgaatggaagagaatccgaagaagaaaatctcaataaatctgaaataagtcaagtgtttgagattgcacttaaacggaacttgcctgtgaatttcgaggtggcccgggagagtggcccaccccacatgaagaactttgtgaccaaggtttcggttggggagtttgtgggggaaggtgaagggaaaagcaagaagatttcaaagaaaaatgccgccatagctgttcttgaggagctgaagaagttaccgcccctgcctgcagttgaacgagtaaagcctagaatcaaaaagaaaacaaaacccatagtcaagccacagacaagcccagaatatggccaggggatcaatccgattagccgactggcccagatccagcaggcaaaaaaggagaaggagccagagtacacgctcctcacagagcgaggcctcccgcgccgcagggagtttgtgatgcaggtgaaggttggaaaccacactgcagaaggaacgggcaccaacaagaaggtggccaagcgcaatgcagccgagaacatgctggagatccttggtttcaaagtcccgcaggcgcagcccaccaaacccgcactcaagtcagaggagaagacacccataaagaaaccaggggatggaagaaaagtaaccttttttgaacctggctctggggatgaaaatgggactagtaataaagaggatgagttcaggatgccttatctaagtcatcagcagctgcctgctggaattcttcccatggtgcccgaggtcgcccaggctgtaggagttagtcaaggacatcacaccaaagattttaccagggcagctccgaatcctgccaaggccacggtaactgccatgatagcccgagagttgttgtatgggggcacctcgcccacagccgagaccattttaaagaataacatctcttcaggccacgtaccccatggacctctcacgagaccctctgagcaactggactatctttccagagtccagggattccaggttgaatacaaagacttccccaaaaacaacaagaacgaatttgtatctcttatcaattgctcctctcagccacctctgatcagccatggtatcggcaaggatgtggagtcctgccatgatatggctgcgctgaacatcttaaagttgctgtctgagttggaccaacaaagtacagagatgccaagaacaggaaacggaccaatgtctgtgtgtgggaggtgctga

SEQ ID NO: 39 - DNA, 1302 bp, Homo sapiens
atggaggtggtggacccgcagcagctgggcatgttcacggagggcgagctgatgtcggtgggtatggacacgttcatccaccgcatcgactccaccgaggtcatctaccagccgcgccgcaagcgggccaagctcatcggcaagtacctgatgggggacctgctgggggaaggctcttacggcaaggtgaaggaggtgctggactcggagacgctgtgcaggagggccgtcaagatcctcaagaagaagaagttgcgaaggatccccaacggggaggccaacgtgaagaaggaaattcaactactgaggaggttacggcacaaaaatgtcatccagctggtggatgtgttatacaacgaagagaagcagaaaatgtatatggtgatggagtactgcgtgtgtggcatgcaggaaatgctggacagcgtgccggagaagcgtttcccagtgtgccaggcccacgggtacttctgtcagctgattgacggcctggagtacctgcatagccagggcattgtgcacaaggacatcaagccggggaacctgctgctcaccaccggtggcaccctcaaaatctccgacctgggcgtggccgaggcactgcacccgttcgcggcggacgacacctgccggaccagccagggctccccggctttccagccgcccgagattgccaacggcctggacaccttctccggcttcaaggtggacatctggtcggctggggtcaccctctacaacatcaccacgggtctgtaccccttcgaaggggacaacatctacaagttgtttgagaacatcgggaaggggagctacgccatcccgggcgactgtggccccccgctctctgacctgctgaaagggatgcttgagtacgaaccggccaagaggttctccatccggcagatccggcagcacagctggttccggaagaaacatcctccggctgaagcaccagtgcccatcccaccgagcccagacaccaaggaccggtggcgcagcatgactgtggtgccgtacttggaggacctgcacggcgcggacgaggacgaggacctcttcgacatcgaggatgacatcatctacactcaggacttcacggtgcccggacaggtcccagaagaggaggccagtcacaatggacagcgccggggcctccccaaggccgtgtgtatgaacggcacagaggcggcgcagctgagcaccaaatccagggcggagggccgggcccccaaccctgcccgcaaggcctgctccgccagcagcaagatccgccggctgtcggcctgcaagcagcagtga

SEQ ID NO: 40 - DNA, 1482 bp, Homo sapiens
atggactttctcctggggaacccgttcagctctccagtgggacagcgcatcgagaaagccacagatggctccctgcagagcgaggactgggccctcaacatggagatctgcgacatcatcaacgagacggaggaaggtcccaaagatgccctccgagcagtaaagaagagaatcgtggggaataagaacttccacgaggtgatgctggctctcacagtcttagaaacctgtgtcaagaactgcgggcaccgcttccacgtgctggtggccagccaggacttcgtggagagtgtgctggtgaggaccatcctgcccaagaacaacccacccaccatcgtgcatgacaaagtgctcaacctcatccagtcctgggctgacgcgttccgcagctcgcccgatctgacaggtgtggtcaccatctatgaggacctgcggaggaaaggcctggagttccccatgactgacctggacatgctgtcacccatccacacaccccagaggaccgtgttcaactcagagacacaatcaggacaggattctgtgggcactgactccagccagcaagaggactctggccagcatgctgcccctctgcccgccccgcccatactctccggtgacacgcccatagcaccaaccccggaacagattgggaagctgcgcagtgagctggagatggtgagtgggaacgtgagggtgatgtcggagatgctgacggagctggtgcccacccaggccgagcccgcagacctggagctgctgcaggagctcaaccgcacgtgccgagccatgcagcagcgggtcctggagctcatccctcagatcgccaatgagcagctgacagaggagctgctcatcgtcaatgacaatctcaacaatgtgttcctgcgccatgaacggtttgaacggttccgaacaggccagaccaccaaggccccaagtgaggccgagccggcagctgacctgatcgacatgggccctgacccagcagccaccggcaacctctcatcccagctggcaggaatgaacctgggctccagcagtgtgagagctggcctgcagtctctggaggcctctggtcgactggaagatgagtttgacatgtttgcgctgacacggggcagctcactggctgaccaacggaaagaggtaaaatacgaagccccccaagcaacagacggcctggctggagccctggacgcccggcagcagagcactggcgcgatcccagtcacccaggcctgcctcatggaggacatcgagcagtggctgtccactgacgtgggtaatgatgcggaagagcctaagggggtcaccagcgaaggtaaatttgacaaattcctggaagaacgggccaaagccgcggaccgattgcccaacctctccagcccctcagctgaggggcccccgggtcccccatctggcccagcgccccggaagaagacccaggagaaagatgatgacatgctgtttgccttatga

SEQ ID NO: 41 - DNA, 1008 bp, Homo sapiens
atggcggcgggggcggctgaggcagctgtagcggccgtggaggaggtcggctcagccgggcagtttgaggagctgctgcgcctcaaagccaagtccctccttgtggtccatttctgggcaccatgggctccacagtgtgcacagatgaacgaagttatggcagagttagctaaagaactccctcaagtttcatttgtgaagttggaagctgaaggtgttcctgaagtatctgaaaaatatgaaattagctctgttcccacttttctgtttttcaagaattctcagaaaatcgaccgattagatggtgcacatgccccagagttgaccaaaaaagttcagcgacatgcatctagtggctccttcctacccagcgctaatgaacatcttaaagaagatctcaaccttcgcttgaagaaattgactcatgctgccccctgcatgctgtttatgaaaggaactcctcaagaaccacgctgtggtttcagcaagcagatggtggaaattcttcacaaacataatattcagtttagcagttttgatatcttctcagatgaagaggttcgacagggactcaaagcctattccagttggcctacctatcctcagctctatgtttctggagagctcataggaggacttgatataattaaggagctagaagcatctgaagaactagatacaatttgtcccaaagctcccaaattagaggaaaggctcaaagtgctgacaaataaagcttctgtgatgctctttatgaaaggaaacaaacaggaagcaaaatgtggattcagcaaacaaattctggaaatactaaatagtactggtgttgaatatgaaacattcgatatattggaggatgaagaagttcggcaaggattaaaagcttactcaaattggccaacataccctcagctgtatgtgaaaggggagctggtgggaggattggatattgtgaaggaactgaaagaaaatggtgaattgctgcctatactgagaggagaaaattaa

SEQ ID NO: 42 - DNA, 1500 bp, Homo sapiens
atgaacggccctgaagatcttcccaagtcctatgactatgaccttatcatcattggaggtggctcaggaggtctggcagctgctaaggaggcagcccaatatggcaagaaggtgatggtcctggactttgtcactcccacccctcttggaactagatggggtcttggaggaacatgtgtgaatgtgggttgcatacctaaaaaactgatgcatcaagcagctttgttaggacaagccctgcaagactctcgaaattatggatggaaagtcgaggagacagttaagcatgattgggacagaatgatagaagctgtacagaatcacattggctctttgaattggggctaccgagtagctctgcgggagaaaaaagtcgtctatgagaatgcttatgggcaatttattggtcctcacaggattaaggcaacaaataataaaggcaaagaaaaaatttattcagcagagagatttctcattgccactggtgaaagaccacgttacttgggcatccctggtgacaaagaatactgcatcagcagtgatgatcttttctccttgccttactgcccgggtaagaccctggttgttggagcatcctatgtcgctttggagtgcgctggatttcttgctggtattggtttagacgtcactgttatggttaggtccattcttcttagaggatttgaccaggacatggccaacaaaattggtgaacacatggaagaacatggcatcaagtttataagacagttcgtaccaattaaagttgaacaaattgaagcagggacaccaggccgactcagagtagtagctcagtccaccaatagtgaggaaatcattgaaggagaatataatacggtgatgctggcaataggaagagatgcttgcacaagaaaaattggcttagaaaccgtaggggtgaagataaatgaaaagactggaaaaatacctgtcacagatgaagaacagaccaatgtgccttacatctatgccattggcgatatattggaggataaggtggagctcaccccagttgcaatccaggcaggaagattgctggctcagaggctctatgcaggttccactgtcaagtgtgactatgaaaatgttccaaccactgtatttactcctttggaatatggtgcttgtggcctttctgaggagaaagctgtggagaagtttggggaagaaaatattgaggtttaccatagttacttttggccattggaatggacgattccgtcaagagataacaacaaatgttatgcaaaaataatctgtaatactaaagacaatgaacgtgttgtgggctttcacgtactgggtccaaatgctggagaagttacacaaggctttgcagctgcgctcaaatgtggactgaccaaaaagcagctggacagcacaattggaatccaccctgtctgtgcagaggtattcacaacattgtctgtgaccaagcgctctggggcaagcatcctccaggctggctgctgaggttaa

SEQ ID NO: 43 - DNA, 3201 bp, Homo sapiens
atgccagtgtttcatacgcgcacgatcgagagcatcctggagccggtggcacagcagatctcccacctggtgataatgcacgaggagggcgaggtggacggcaaagccattcctgacctcaccgcgcccgtggccgccgtgcaggcggccgtcagcaacctcgtccgggttggaaaagagactgttcaaaccactgaggatcagattttgaagagagatatgccaccagcatttattaaggttgagaatgcttgcaccaagcttgtccaggcagctcagatgcttcagtcagacccttactcagtgcctgctcgagattatctaattgatgggtcaaggggcatcctctctggaacatcagacctgctccttaccttcgatgaggctgaggtccgtaaaattattagagtttgcaaaggaattttggaatatcttacagtggcagaggtggtggagactatggaagatttggtcacttacacaaagaatcttgggccaggaatgactaagatggccaagatgattgacgagagacagcaggagctcactcaccaggagcaccgagtgatgttggtgaactcgatgaacaccgtgaaagagttgctgccagttctcatttcagctatgaagatttttgtaacaactaaaaactcaaaaaaccaaggcatagaggaagctttaaaaaatcgcaattttactctagaaaaaatgagtgctgaaattaatgagataattcgtgtgttacaactcacctcttgggatgaagatgcctgggccagcaaggacactgaagccatgaagagagcattggcctccatagactccaaactgaaccaggccaaaggttggctccgtgaccctagtgcctccccaggggatgctggtgagcaggccatcagacagatcttagatgaagctggaaaagttggtgaactctgtgcaggcaaagaacgcagggagattctgggaacttgcaaaatgctagggcagatgactgatcaagtggctgacctccgtgccagaggacaaggatcctcaccggtggccatgcagaaagctcagcaggtatctcagggtctggatgtgctcacagcaaaagtggaaaatgcagctcgcaagctggaagccatgaccaactcaaagcagagcattgcaaagaagatcgatgctgctcagaactggcttgcagatccaaatggtggaccggaaggagaagagcagattcgaggtgctttggctgaagctcggaaaatagcagaattatgtgatgatcctaaagaaagagatgacattctacgttcccttggggaaatatctgctctgacttctaaattagcagatctacgaagacaggggaaaggagattctccagaggctcgagccttggccaaacaggtggccacggccctgcagaacctgcagaccaaaaccaaccgggctgtggccaacagcagaccggccaaagcagctgtacaccttgagggcaagattgagcaagcacagcggtggattgataatcccacagtggatgaccgtggagtcggtcaggctgccatccgggggcttgtggccgaagggcatcgtctggctaatgttatgatggggccttatcggcaagatcttctcgccaagtgtgaccgagtggaccagctgacagcccagctggctgacctggctgccagaggggaaggggagagtcctcaggcacgagcacttgcatctcagctccaagactccttaaaggatctaaaagctcggatgcaggaggccatgactcaggaagtgtcagatgttttcagcgataccacaactcccatcaagctgttggcagtggcagccacggcgcctcctgatgcgcctaacagggaagaggtatttgatgagagggcagctaactttgaaaaccattcaggaaagcttggtgctacggccgagaaggcggctgcggttggtactgctaataaatcaacagtggaaggcattcaggcctcagtgaagacggcccgagaactcacaccccaggtggtctcggctgctcgtatcttacttaggaaccctggaaatcaagctgcttatgaacattttgagaccatgaagaaccagtggatcgataatgttgaaaaaatgacagggctggtggacgaagccattgataccaaatctctgttggatgcttcagaagaagcaattaaaaaagacctggacaagtgcaaggtagctatggccaacattcagcctcagatgctggttgctggggcaaccagtattgctcgtcgggccaaccggatcctgctggtggctaagagggaggtggagaattccgaggatcccaagttccgtgaggctgtgaaagctgcctctgatgaattgagcaaaaccatctccccgatggtgatggatgcaaaagctgtggctggaaacatttccgaccctggactgcaaaagagcttcctggactcaggatatcggatcctgggagctgtggccaaggtcagagaagccttccaacctcaggagcctgacttcccgccgcctccaccagaccttgaacaactccgactaacagatgagcttgctcctcccaaaccacctctgcctgaaggtgaggtccctccacctaggcctccaccaccagaggaaaaggatgaagagttccctgagcagaaggccggggaggtgattaaccagccaatgatgatggctgccagacagctccatgatgaagctcgcaaatggtccagcaagggcaatgacatcattgcagcagccaagcgcatggctctgctgatggctgagatgtctcggctggtaagagggggcagtggtaccaagcgggcactcattcagtgtgccaaggacatcgccaaggcctcagatgaggtgactcggttggccaaggaggttgccaagcagtgcacagataaacggattagaaccaacctcttacaggtatgtgagcgaatcccaaccataagcacccagctcaaaatcctgtccacagtgaaggccaccatgctgggccggaccaacatcagtgatgaggagtctgagcaggccacagagatgctggttcacaatgcccagaacctcatgcagtctgtgaaggagactgtgcgggaagctgaagctgcttcaatcaaaattcgaacagatgctggatttacactgcgctgggttagaaagactccctggtaccagtag

SEQ ID NO: 44 - DNA, 600 bp, Homo sapiens
atggcgtcgggcagcgggacaaaaaacttggactttcgccgaaagtgggacaaagatgaatatgagaaactcgccgagaagaggctcacggaagagagagaaaagaaagatggaaaaccagtgcagcctgtcaagcgagagcttttacggcatagggactacaaggtggacttggaatccaagcttgggaagacaattgtcattaccaagacaacccctcaatctgagatgggaggatattactgcaatgtctgtgactgtgtggtgaaggactccatcaactttctggatcacattaatggaaagaaacatcagagaaacctgggcatgtctatgcgtgtggaacgttccaccctggatcaggtgaagaaacgttttgaggtcaacaagaagaagatggaagagaagcagaaggattatgattttgaggaaaggatgaaggagctcagagaagaggaggaaaaggccaaagcgtacaagaaagagaaacagaaggagaagaaaaggagggctgaggaggacttgacatttgaggaggacgatgagatggcagctgtgatgggcttctctggctttggttccaccaagaagagttactga

SEQ ID NO: 45 - DNA, 1422 bp, Homo sapiens
atgatgaccaaggtactaggcatggccccagttctgggccctaggcctccacaggagcaggtggggcctctgatggtaaaagtcgaggagaaagaagagaaaggcaagtaccttcctagcctggagatgttccgccagcgcttcaggcagtttgggtaccatgatacccctggaccccgagaggccctgagccaactccgggtgctctgctgtgagtggctgaggcccgagatccacaccaaggagcagatcctggagctactggtgctggagcagttcctgaccatcctgccccaggagctccaggcctgggtgcaggagcattgcccggagagcgctgaagaggctgtcactctcctcgaagatctggagcgggaactggatgagccaggacaccaggtctcaactcctccaaacgaacagaaaccggtgtgggagaagatatcctcctcaggaactgcaaaggaatccccgagcagcatgcagccacagcccttggagaccagtcacaaatacgagtcttgggggcccctgtacatccaagagtctggtgaggagcaggagttcgctcaagatccaagaaaggtccgagattgcagattgagtacccagcacgaggaatcagcagatgagcagaaaggttctgaagcagaggggctcaaaggggatataatttctgtgattatcgccaataaacctgaggccagcttagagaggcagtgcgtaaaccttgaaaatgaaaaaggaacaaaaccccctcttcaagaggcaggctccaagaaaggtagagaatcagttcctactaaacctaccccaggagagagacgttatatatgtgctgaatgtggcaaagcctttagtaatagctcaaatctcaccaaacacaggagaacacacactggggagaaaccttacgtgtgcaccaagtgtgggaaagctttcagccacagctcaaacctcaccctccactacagaacacacttggtggaccggccctatgactgtaagtgtggaaaagcttttgggcagagctcagaccttcttaaacatcagagaatgcacacagaagaggcgccatatcagtgcaaagattgtggcaaggctttcagcgggaaaggcagcctcattcgtcactatcggatccacactggggagaagccttatcagtgtaacgaatgtgggaagagcttcagtcagcatgcgggcctcagctcccaccagagactccacaccggagagaagccatataagtgtaaggagtgtgggaaagccttcaaccacagctccaacttcaataaacaccacagaatccacaccggggaaaagccctactggtgtcatcactgtggaaagaccttctgtagcaagtccaatctttccaaacatcagcgagtccacactggagagggagaagcaccgtaa

(19) United States
(12) Patent Application Publication
(10) Pub. No.: US 2011/0207613 A1
(43) Pub. Date: Aug. 25, 2011

(54) BIOMARKERS FOR LUPUS

(75) Inventors: Martin Johannes Ebner, Berkshire (GB); Colin Henry Wheeler, Berkshire (GB); Rachel Alison Fallon, Berkshire (GB); Sarah Paula Joyce, Berkshire (GB); Jens-Oliver Koopman, Berkshire (GB); Michael Bernard McAndrew, Berkshire (GB); Nicholas Ian Workman, Berkshire (GB)

(73) Assignee: Sense Proteomic Limited

(21) Appl. No.: 12/997,031

(22) PCT Filed: Jun. 11, 2009
(86) PCT No.: PCT/GB09/01457

§ 371 Date: Apr. 21, 2011

(30) Foreign Application Priority Data
Jun. 11, 2008 (GB) 0810709.6

(51) Int. Cl. C40B 30/00; G01N 33/573; G01N 33/566; C40B 40/10

(52) U.S. Cl. 506/7; 435/7.4; 436/501; 506/18

(57) ABSTRACT

The present invention provides biomarkers valuable in diagnosing or detecting a susceptibility to lupus. The present invention also provides panels of biomarkers valuable in diagnosing or detecting a susceptibility to lupus with good specificity and selectivity. Said biomarkers and panels of biomarkers are also useful in monitoring the progression or regression of lupus, or monitoring the progression to a flare of the disease or transition from a flare into remission or monitoring the efficacy of a therapeutic agent to lupus.

### FIELD OF THE INVENTION

The present invention relates to novel biomarkers for lupus and methods and kits for their use. Also provided are vaccines for the treatment or prevention of lupus.

### BACKGROUND

Systemic lupus erythematosus (SLE) or lupus is a chronic autoimmune disease that can affect the joints and almost every major organ in the body, including heart, kidneys, skin, lungs, blood vessels, liver, and the nervous system. As in other autoimmune diseases, the body's immune system attacks the body's own tissues and organs, leading to inflammation. A person's risk to develop lupus appears to be determined mainly by genetic factors, but environmental factors, such as infection or stress may trigger the onset of the disease. The course of lupus varies, and is often characterised by alternating periods of flares, i.e. increased disease activity, and periods of remission.

It has been estimated that in the US, about 1 million people suffer from lupus, world-wide, conservative estimates indicate about 4 million lupus patients. Lupus occurs about 9 times more frequently in women than in men. Although lupus can occur at any age, it is most common in women of childbearing age.

There is presently no cure for lupus. However, there are means of reducing and dealing with flares, including drugs such as anti-inflammatory drugs, alternative medicines or life-style changes. The treatment regimes will depend on the severity of the disease, and the responsiveness of the patient. Disease-modifying antirheumatic drugs can be used preventively to reduce the incidence of flares. When flares occur, they are often treated with corticosteroids.

As mentioned above, lupus patients can present with a variety of diverse symptoms, and is therefore difficult to diagnose. A number of tests are used in order to make a diagnosis, including antinuclear antibody test (ANA), tests for other auto-antibodies such as anti-DNA antibodies, tests for serum complement levels, urine analysis, looking for elevated protein levels in urine, and sometimes biopsies of an affected organ are used. However, these tests do not necessarily give a definitive diagnosis; for example, a positive ANA test can occur due to infections or rheumatic diseases, and even healthy people without lupus can test positive. The sensitivity and specificity for the ANA test are 93% and 57% (Habash-Bseiso,D Clin Med Res. 2005 August; 3(3): 190-193). Lupus diagnosis often takes 7 to 10 years using current methods.

There is therefore a need for new or improved biomarkers for lupus that can be used in methods of diagnosing lupus, for the early detection of lupus, subclinical or presymptomatic lupus or a predisposition to lupus, or for monitoring the progression of lupus or the likelihood to transition from remission to flare or vice versa, or the efficacy of a therapeutic treatment thereof.

### SUMMARY OF THE INVENTION

It has now been found that the presence of certain auto-antibodies or combinations of auto-antibodies in a patient, or a significant increase in the number or concentration of such auto-antibodies or combinations of auto-antibodies provide useful biomarkers for lupus. Early results indicate that the disclosed invention will also distinguish lupus from other diseases.

Therefore the present invention provides biomarkers valuable in diagnosing or detecting a susceptibility to lupus. The present invention also provides panels of biomarkers valuable in diagnosing lupus or detecting a susceptibility to lupus. The present disclosure includes panels with good specificity and/or selectivity as compared to prior art methods. Said biomarkers and panels of biomarkers are also useful in monitoring the progression or regression of lupus, or monitoring the progression to a flare of the disease or transition from a flare into remission or monitoring the efficacy of a therapeutic agent to lupus.

In one embodiment, the present invention provides methods/uses and products (e.g. biomarkers, kits, vaccines, panels and methods of using the same) which employ ZMAT2 (including variants thereof (see discussion below)) as an auto-antigen, optionally with at least 1, 2, 3, 4, 5, 7, 9, 10, 15 or 20 other auto-antigens (e.g. as recited in Tables 1, 2, 3, 4 or 5) also being used.

Suitably the at least 1, 2, 3, 4, 5, 7, 9, 10, 15, 20 or 25 other auto-antigens that are used are selected from the group consisting of:
(a) BPY2IP1 (MAP1S protein); CEBPG (CCAAT/enhancer binding protein (C/EBP), gamma); E1B-AP5 (E1B-55 kDa-associated protein 5); FUS (Fusion (involved in t(12;16) in malignant liposarcoma)); HAGH (Hydroxyacylglutathione hydrolase); HMG20B (High-mobility group 20B); HOXB6 (Homeo box B6, transcript variant 2); KRT8 (Keratin 8); LIN28 (Lin-28 homolog (C. elegans)); NDUFV3 (NADH dehydrogenase (ubiquinone) flavoprotein 3, 10 kDa); PABPC1 (Poly(A) binding protein, cytoplasmic 1); PHLDA1 (Pleckstrin homology-like domain, family A, member 1); PIAS2 (Msx-interacting-zinc finger, transcript variant alpha); RAB11FIP3 (RAB11 family interacting protein 3 (class II)); RAN (RAN, member RAS oncogene family); RARA (Retinoic acid receptor alpha); RBMS1 (RNA binding motif, single stranded interacting protein 1, transcript variant); RDBP (RD RNA binding protein); RNF12 (Ring finger protein 12, transcript variant 1); RUFY1 (RUN and FYVE domain containing 1); SMN1 (Survival of motor neuron 1, telomeric); SRPK1 (SFRS protein kinase 1); SSNA1 (Sjogren's syndrome nuclear autoantigen 1); STAU. (Staufen, RNA binding protein (Drosophila), transcript variant T3); STK11 (Serine/threonine kinase 11 (Peutz-Jeghers syndrome)); TOM1 (Target of myb1 (chicken)); TXNL2 (Thioredoxin-like, clone MGC:12349); TXNRD1 (Thioredoxin reductase 1, transcript variant 5); and ZNF38 (Zinc finger protein 38); or
(b) ASPSCR1 (alveolar soft part sarcoma chromosome region, candidate 1);

CEBPG (CCAAT/enhancer binding protein (C/EBP), gamma); DDX55 (DEAD (Asp-Glu-Ala-Asp) box polypeptide 55); DOM3Z (dom-3 homolog Z (C. elegans)); E1B-AP5 (E1B-55 kDa-associated protein 5) FUS (Fusion (involved in t(12;16) in malignant liposarcoma)); HMG20B (High-mobility group 20B); KRT8 (Keratin 8); L1N28 Lin-28 homolog (C. elegans)); LNX (ligand of numb-protein X); MAP1S (MAP1S protein); PABPC1 (Poly(A) binding protein, cytoplasmic 1); PHLDA1 (Pleckstrin homology-like domain, family A, member 1); PIAS2 (Msx-interacting-zinc finger, transcript variant alpha); PRKCBP1 (protein kinase C binding protein 1); PRKRA (protein kinase, interferon-inducible double stranded RNA dependent activator); RARA (Retinoic acid receptor, alpha); RDBP (RD RNA binding protein); RPL30 inducible kinase); SRPK1 (SFRS protein kinase 1); STAU1 (Staufen, RNA binding protein (Drosophila), transcript variant T3); TXNL2 (Thioredoxin-like, clone MGC:12349); VCL (vinculin); ZMAT2 (Zinc finger, matrin type 2); and ZNF38. (Zinc finger protein 38).

In addition to ZMAT2 and the at least 1, 2, 3, 4, 5, 7, 9, 10, 15, 20 or 25 other auto-antigens (e.g. as selected from group (a) or (b) above), one or more further auto-antigens may be employed wherein said one or more further auto-antigens are selected from the group consisting of: IFI16 (Interferon, gamma-inducible protein 16); PSME3 (Proteasome (prosome, macropain) activator subunit 3 (PA28 gamma; Ki), transc); RALBP1 (RalA-binding protein 1); SSA2 (Sjogren syndrome antigen A2 (60 kDa, ribonucleoprotein autoantigen SS-A/Ro)); and BANK1 (B-cell scaffold protein with ankyrin repeats 1).

Thus, it will be appreciated that the methods of the invention may comprise testing a patient sample against ZMAT2/a combination of auto-antigens which combination may be as outlined above (including variants thereof). In this way, the presence, absence or a modulated level of auto-antibodies in the sample may be detected. In some embodiments the auto-antigens are provided/used in the form of a panel and methods of using these panels as well as the panels themselves form part of the present invention.

Examples of particular methods/uses and products employing ZMAT2/a combination of auto-antigens as outlined above will be apparent from the description and the appended claim set of this application. Also, specific examples of methods/uses and products as described herein may be modified so as to employ any particular combination of auto-antigens that can be derived from the above discussion of the methods/uses and products of the present invention.

The methods, biomarkers, kits and panels of the present invention may be used alone or in combination with (e.g. before, after or concurrently with) one or more other diagnostic/prognostic indicators or techniques. Such a combination may for instance be used to to: (i) diagnose lupus; (ii) detect a susceptibility to lupus; (iii) monitor the progression or regression of lupus; (iv) monitor the progression to a flare of the disease or transition from a flare into remission; or (v) monitor the efficacy of a therapeutic agent to lupus.

Examples of diagnostic/prognostic indicators or techniques which may be employed in combination with the disclosure of the present invention can include those which have utility in the diagnosis/prognosis of lupus. Examples may include: the presence of symptoms characteristic or indicative of lupus; a complete or incomplete SLEDAI (Systemic Lupus Erythematosus Disease Activity Index) score; anti-nuclear antibody (ANA) blood test; tests for other auto-antibodies such as anti-DNA antibodies; tests for serum complement levels; urine analysis (e.g. looking for elevated protein levels in urine); the use of one or more other biomarkers; anti-ds DNA test; blood counts; and biopsy analysis of an affected organ.

The present invention provides a method useful in diagnosing a patient as having or being predisposed to developing lupus, comprising testing a sample from the patient against a panel of auto-antigens to detect the presence, absence or a modulated level of auto-antibodies in the sample, wherein the panel of auto-antigens comprises:

(a) the auto-antigens listed in Table 2,

(b) the panel of (a) in which up to 5 auto-antigens are deleted or replaced, or

(c) the panel of (a) in which up to 6, 7, 8, 9 or 10 ore more auto-antigens are deleted or replaced.

As will be appreciated from the definition of “diagnosis” (see below), the above method may be useful in providing a positive or negative diagnosis of lupus (or a predisposition thereto).

The auto-antigens of Table 2 are ZMAT2 (Zinc finger, matrin type 2); ASPSCR1 (alveolar soft part sarcoma chromosome region, candidate 1); BANK1 B-cell (scaffold protein with ankyrin repeats 1); CEBPG (CCAAT/enhancer binding protein (C/EBP), gamma); DDX55 (DEAD (Asp-Glu-Ala-Asp) box polypeptide 55); DOM3Z (dom-3 homolog Z (C. elegans)); E1B-AP5 (E1B-55 kDa-associated protein 5) FUS (Fusion (involved in t(12;16) in malignant liposarcoma)); HMG20B (High-mobility group 20B); IFI16 (Interferon, gamma-inducible protein 16); KRT8 (Keratin 8); L1N28 Lin-28 homolog (C. elegans)); LNX (ligand of numb-protein X); MAP1S (MAP1S protein); PABPC1 (Poly(A) binding protein, cytoplasmic 1); PHLDA1 (Pleckstrin homology-like domain, family A, member 1); PIAS2 (Msx-interacting-zinc finger, transcript variant alpha); PRKCBP1 (protein kinase C binding protein 1); PRKRA (protein kinase, interferon-inducible double stranded RNA dependent activator); PSME3 (Proteasome (prosome, macropain) activator subunit 3 (PA28 gamma; Ki)); RALBP1 (RalA binding protein 1); RARA (Retinoic acid receptor, alpha); RDBP (RD RNA binding protein); RPL30 (ribosomal protein L30); RPL31 (ribosomal protein L31); SNK (serum-inducible kinase); SRPK1 (SFRS protein kinase 1); SSA2 (Sjogren syndrome antigen A2 (6010a, ribonucleoprotein autoantigen SS-A/Ro)); STAU1 (Staufen, RNA binding protein (Drosophila), transcript variant T3); TXNL2 (Thioredoxin-like, clone MGC:12349); VCL (vinculin); ZNF38 (Zinc finger protein 38).

The present invention provides a method useful in diagnosing a patient as having or being predisposed to developing lupus, comprising testing a sample from the patient against a panel of auto-antigens to detect the presence, absence or a modulated level of auto-antibodies in the sample, wherein the panel of auto-antigens comprises:

(a) the auto-antigens listed in Table 1,

(b) the panel of (a) in which up to 5 auto-antigens are deleted or replaced, or

(c) the panel of (a) in which up to 6, 7, 8, 9 or 10 or more auto-antigens are deleted or replaced.

The auto-antigens of Table 1 are ZMAT2 (Zinc finger, matrin type 2); BANK1 (B-cell scaffold protein with ankyrin repeats 1); BPY2IP1 (MAP1S protein); CEBPG (CCAAT/enhancer binding protein (C/EBP), gamma); E1B-AP5 (E1B-55 kDa-associated protein 5); FUS (Fusion (involved in t(12;16) in malignant liposarcoma)); HAGH (Hydroxyacylglutathione hydrolase); HMG20B (High-mobility group 20B); HOXB6 (Homeo box B6, transcript variant 2); IFI16 (Interferon, gamma-inducible protein 16); KRT8 (Keratin 8); L1N28 (Lin-28 homolog (C. elegans)); NDUFV3 (NADH dehydrogenase (ubiquinone) flavoprotein 3, 10 KDa); PABPC1 (Poly(A) binding protein, cytoplasmic 1); PHLDA1 (Pleckstrin homology-like domain, family A, member 1); PIAS2 (Msx-interacting-zinc finger, transcript variant alpha); RAB11FIP3 (RAB11 family interacting protein 3 (class II)); RALBP1 (RalA-binding protein 1); RAN (RAN, member RAS oncogene family); RARA (Retinoic acid receptor alpha); RBMS1 (RNA binding motif, single stranded interacting protein 1, transcript variant); RDBP (RD RNA binding protein); RNF12 (Ring finger protein 12, transcript variant 1); RUFY1 (RUN and FYVE domain containing 1); SMN1 (Survival of motor neuron 1, telomeric); SRPK1 (SFRS protein kinase 1); SSNA1 (Sjogren's syndrome nuclear autoantigen 1); STAU (Staufen, RNA binding protein (Drosophila), transcript variant T3); STK11 (Serine/threonine kinase 11 (Peutz-Jeghers syndrome)); TOM1 (Target of myb1 (chicken)); TXNL2 (Thioredoxin-like, clone MGC:12349); TXNRD1 (Thioredoxin reductase 1, transcript variant 5); ZNF38 (Zinc finger protein 38), PSME3 (Proteasome (prosome, macropain) activator subunit 3 (PA28 gamma; Ki), transc), and SSA2 (Sjogren syndrome antigen A2 (60 kDa, ribonucleoprotein autoantigen SS-A/Ro)).

The present invention provides a method useful in diagnosing a patient as having or being predisposed to developing lupus, comprising testing a sample from the patient against a panel of auto-antigens to detect the presence, absence or a modulated level of auto-antibodies in the sample, wherein the panel of auto-antigens comprises:

(a) the auto-antigens listed in Table 4,

(b) the panel of (a) in which up to 5 auto-antigens are deleted or replaced, or

(c) the panel of (a) in which up to 6, 7, 8, 9 or 10 or more auto-antigens are deleted or replaced.

The auto-antigens of Table 4 are ZMAT2 (Zinc finger, matrin type 2); BANK1 (B-cell scaffold protein with ankyrin repeats 1); BPY2IP1 or MAP1S (MAP1S protein); CEBPG (CCAAT/enhancer binding protein (C/EBP), gamma); E1B-AP5 (E1B-55 kDa-associated protein 5); FUS (Fusion (involved in t(12;16) in malignant liposarcoma)); HMG20B (High-mobility group 20B); IFI16 (Interferon, gamma-inducible protein 16); KRT8 (Keratin 8); LIN28 (Lin-28 homolog (C. elegans)); PABPC1 (Poly(A) binding protein, cytoplasmic 1); PHLDA1 (Pleckstrin homology-like domain, family A, member 1); PIAS2 (Msx-interacting-zinc finger, transcript variant alpha); PSME3 (Proteasome (prosome, macropain) activator subunit 3 (PA28 gamma; Ki)); RALBP1 (RalA binding protein 1); RARA (Retinoic acid receptor, alpha); RDBP (RD RNA binding protein); SRPK1 (SFRS protein kinase 1); SSA2 (Sjogren syndrome antigen A2 (60 kDa, ribonucleoprotein autoantigen SS-A/Ro)); STAU (Staufen, RNA binding protein (Drosophila), transcript variant T3); TXNL2 (Thioredoxin-like, clone MGC:12349); ZNF38 (Zinc finger protein 38).

The present invention provides a method useful in diagnosing a patient as having or being predisposed to developing lupus, comprising testing a sample from the patient against a panel of auto-antigens to detect the presence, absence or a modulated level of auto-antibodies in the sample, wherein the panel of auto-antigens comprises:

(a) the auto-antigens listed in Table 3,

(b) the panel of (a) in which up to 5 auto-antigens are deleted or replaced, or

(c) the panel of (a) in which up to 10, 15, 20 or 25 or more auto-antigens are deleted or replaced.

The auto-antigens of Table 3 are ZMAT2 (Zinc finger, matrin type 2); ASPSCR1 (alveolar soft part sarcoma chromosome region, candidate 1); BANK1 (B-cell scaffold protein with ankyrin repeats 1); BPY2IP1 or MAP1S (MAP1S protein); CEBPG (CCAAT/enhancer binding protein (C/EBP), gamma); DDX55 (DEAD (Asp-Glu-Ala-Asp) box polypeptide 55); DOM3Z (dom-3 homolog Z (C. elegans)); E1B-AP5 (E1B-55 kDa-associated protein 5); FUS (Fusion (involved in t(12;16) in malignant liposarcoma)); HAGH (Hydroxyacylglutathione hydrolase); HMG20B (High-mobility group 20B); HOXB6 (Homeo box B6, transcript variant 2); IFI16 (Interferon, gamma-inducible protein 16); KRT8 (Keratin 8); L1N28 (Lin-28 homolog (C. elegans)); LNX (ligand of numb-protein X); NDUFV3 (NADH dehydrogenase (ubiquinone) flavoprotein 3, 10 kDa); PABPC1 (Poly(A) binding protein, cytoplasmic 1); PHLDA1 (Pleckstrin homology-like domain, family A, member 1); PIAS2 (Msx-interacting-zinc finger, transcript variant alpha); PRKCBP1 (protein kinase C binding protein 1); PRKRA (protein kinase, interferon-inducible double stranded RNA dependent activator); PSME3 (Proteasome (prosome, macropain) activator subunit 3 (PA28 gamma; Ki)); RAB11FIP3 (RAB11 family interacting protein 3 (class II)); RALBP1 (RalA binding protein 1); RAN (RAN, member RAS oncogene family); RARA (Retinoic acid receptor, alpha); RBMS1 (RNA binding motif, single stranded interacting protein 1, transcript variant); RDBP (RD RNA binding protein); RNF12 (Ring finger protein 12, transcript variant 1); RPL30 (ribosomal protein L30); RPL31 (ribosomal protein L31); RUFY1 (RUN and FYVE domain containing 1); SMN1 (Survival of motor neuron 1, telomeric); SNK (serum-inducible kinase); SRPK1 (SFRS protein kinase 1) ;SSA2 (Sjogren syndrome antigen A2 (60 kDa, ribonucleoprotein autoantigen SS-A/Ro)); SSNA1 Sjogren's syndrome nuclear autoantigen 1); STAU (Staufen, RNA binding protein (Drosophila), transcript variant T3); STK11 (Serine/threonine kinase 11 (Peutz-Jeghers syndrome)); TOM1 (Target of myb1 (chicken)); TXNL2 (Thioredoxin-like, clone MGC:12349); TXNRD1 (Thioredoxin reductase 1, transcript variant 5); VCL (Vinculin); ZNF38 (Zinc finger protein 38).

The present invention also provides a method of diagnosing a patient as having or being predisposed to developing lupus, comprising detecting in a sample taken from said patient one or more auto-antibodies that bind an auto-antigen selected from the group consisting of the auto-antigens listed in Table 3 (e.g. ZMAT2), wherein the presence of said one or more auto-antibodies, or an increase in the concentration of said one or more auto-antibodies, indicates that the patient suffers from or is predisposed to develop lupus.

The present invention further provides a method useful in diagnosing a patient as having or being predisposed to developing lupus, comprising testing a sample from the patient against a panel of auto-antigens to detect the presence, absence or a modulated level of auto-antibodies in the sample, wherein the panel of auto-antigens comprises at least 10 auto-antigens selected from Table 3 or from Table 1 or from Table 2.

Preferably the panel comprises at least 15 or 20 auto-antigens and/or preferably the panel comprises ZMAT2.

In one embodiment the at least 10, 15 or 20 auto-antigens are selected from the group consisting of:
(i) ZMAT2 (Zinc finger, matrin type 2); BPY2IP1 (MAP1S protein); CEBPG (CCAAT/enhancer binding protein (C/EBP), gamma); E1B-AP5 (E1B-55 kDa-associated protein 5); FUS (Fusion (involved in t(12;16) in malignant liposarcoma)); HAGH (Hydroxyacylglutathione hydrolase); HMG20B (High-mobility group 20B); HOXB6 (Homeo box B6, transcript variant 2); KRT8 (Keratin 8); LIN28 (Lin-28 homolog (C. elegans)); NDUFV3 (NADH dehydrogenase (ubiquinone) flavoprotein 3, 10 kDa); PABPC1 (Poly(A) binding protein, cytoplasmic 1); PHLDA 1 (Pleckstrin homology-like domain, family A, member 1); PIAS2 (Msx-interacting-zinc finger, transcript variant alpha); RAB11FIP3 (RAB11 family interacting protein 3 (class II)); RAN (RAN, member RAS oncogene family); RARA (Retinoic acid receptor alpha); RBMS1 (RNA binding motif, single stranded interacting protein 1, transcript variant); RDBP (RD RNA binding protein); RNF12 (Ring finger protein 12, transcript variant 1); RUFY1 (RUN and FYVE domain containing 1); SMN1 (Survival of motor neuron 1, telomeric); SRPK1 (SFRS protein kinase 1); SSNA1 (Sjogren's syndrome nuclear autoantigen 1); STAU (Staufen, RNA binding protein (Drosophila), transcript variant T3); STK11 (Serine/threonine kinase 11 (Peutz-Jeghers syndrome)); TOM1 (Target of myb1 (chicken)); TXNL2 (Thioredoxin-like, clone MGC:12349); TXNRD1 (Thioredoxin reductase 1, transcript variant 5); and ZNF38 (Zinc finger protein 38); or
(ii) ZMAT2 (Zinc finger, matrin type 2); ASPSCR1 (alveolar soft part sarcoma chromosome region, candidate 1); CEBPG (CCAAT/enhancer binding protein (C/EBP), gamma); DDX55 (DEAD (Asp-Glu-Ala-Asp) box polypeptide 55); DOM3Z (dom-3 homolog Z (C. elegans)); E1B-AP5 (E1B-55 kDa-associated protein 5) FUS (Fusion (involved in t(12;16) in malignant liposarcoma)); HMG20B (High-mobility group 20B); KRT8 (Keratin 8); LIN28 Lin-28 homolog (C. elegans)); LNX (ligand of numb-protein X); MAP1S (MAP1S protein); PABPC1 (Poly(A) binding protein, cytoplasmic 1); PHLDA1 (Pleckstrin homology-like domain, family A, member 1); PIAS2 (Msx-interacting-zinc finger, transcript variant alpha); PRKCBP1 (protein kinase C binding protein 1); PRKRA (protein kinase, interferon-inducible double stranded RNA dependent activator); RARA (Retinoic acid receptor, alpha); RDBP (RD RNA binding protein); RPL30 (ribosomal protein L30); RPL31 (ribosomal protein L31); SNK (serum-inducible kinase); SRPK1 (SFRS protein kinase 1); STAU1 (Staufen, RNA binding protein (Drosophila), transcript variant T3); TXNL2 (Thioredoxin-like, clone MGC:12349); VCL (vinculin); and ZNF38 (Zinc finger protein 38).

Conveniently in the methods of the present invention detecting the presence or an increased level of at least two auto-antibodies provides information useful for diagnosis, preferably detecting the presence of an increased level of at least three, preferably at least four and more preferably at least five auto-antibodies provides information useful for diagnosis.

Advantageously the sample from the patient used in the methods of the present invention is a body fluid, optionally selected from blood, serum, plasma, saliva, lymphatic fluid, wound secretion, urine, faeces, mucus or cerebrospinal fluid (CSF). Typically, the sample may be serum or plasma.

A further aspect of the present invention provides a method of monitoring the progression or regression of lupus, or monitoring the progression to a flare of the disease or transition from a flare into remission, comprising (a) performing methods described above, repeating step (a) using a second sample obtained from the patient at a time later than the first sample; and (c) comparing the auto-antibodies detected in said first and second samples; wherein a change in the level or presence or absence of the auto-antibodies detected between the first and second samples indicates the progression or regression of lupus, or the progression to a flare, or transition from a flare into remission.

Another aspect of the present invention provides a method of monitoring the efficacy in a patient of a therapeutic agent to lupus, comprising (a) performing methods described above (b) administering the therapeutic agent to the patient; (c) performing again method described above using a second sample from the patient taken after the administration of the therapeutic agent; and (d) comparing the auto-antibodies detected in said first and second samples; wherein a change in the level or presence or absence of the auto-antibodies detected in the first and second sample is indicative of the efficacy of the therapeutic agent to lupus.

The present invention also provides a panel of auto-antigens as used in any of the methods described herein. In one embodiment, the present invention provides a panel of auto-antigens, wherein said panel comprises:

(a) the auto-antigens listed in Table 2,

(b) the panel of (a) in which up to 5 auto-antigens are deleted or replaced, or

(c) the panel of (a) in which up to 6, 7, 8, 9 or 10 ore more auto-antigens are deleted or replaced;

(ii) (a) the auto-antigens listed in Table 1,

(b) the panel of (a) in which up to 5 auto-antigens are deleted or replaced, or

(c) the panel of (a) in which up to 6, 7, 8, 9 or 10 or more auto-antigens are deleted or replaced;

(iii) (a) the auto-antigens listed in Table 4,

(b) the panel of (a) in which up to 5 auto-antigens are deleted or replaced, or

(c) the panel of (a) in which up to 6, 7, 8, 9 or 10 or more auto-antigens are deleted or replaced; or

(iv) (a) the auto-antigens listed in Table 3,

(b) the panel of (a) in which up to 5 auto-antigens are deleted or replaced, or

(c) the panel of (a) in which up to 10, 15, 20 or 25 or more auto-antigens are deleted or replaced.

As will be appreciated from the description of the invention, other panels of auto-antigens have also been described for use in the methods of the present invention and these panels may also form part of the present invention. In a preferred embodiment, a panel of the invention comprises ZMAT2.

In one embodiment a panel of the invention is for use in: (i) diagnosing lupus or determining the severity thereof; (ii) detecting a susceptibility to lupus; (iii) monitoring the progression or regression of lupus; (iv) monitoring the progression to a flare of the disease or transition from a flare into remission; or (v) monitoring the efficacy of a therapeutic agent to lupus.

### FIGURES

FIG. 1 is a cartoon illustrating a method for the detection of auto-antibodies in serum.

### DESCRIPTION

As explained further in the Examples below, in order to elucidate biomarkers valuable in diagnosing or detecting susceptibility to lupus, the inventors developed microarrays of antigens suitable for detecting antibodies in samples. Subsequently, a large number of body fluid samples obtained from individuals positively diagnosed as having lupus with existing methods were analysed for the presence of auto-antibodies binding to auto-antigens immobilized on microarrays. Data analysis of the results obtained allowed detection of useful biomarkers for lupus.

A first data analysis methodology allowed detection of a set of 35 biomarkers, auto-antigens to which the patient serum samples contained auto-antibodies, and which are set out in Table 1 below.

A second data analysis methodology allowed detection of a set of 32 biomarkers which are set out in Table 2 below.

Between the two analysis methods a total of 45 useful biomarkers were identified which are set out in Table 3 below. The first and second analysis methods identified 22 biomarkers in common These shared biomarkers are set out in Table 4 below. The biomarkers which were unique to each set are set out in Table 5 below.

In one embodiment of the invention, modified versions of Tables 1, 2, 3, 4 or 5 are envisaged in which 1, 2, 3, 4 or all 5 of the following markers are removed from the Table: IFI16 (Interferon, gamma-inducible protein 16); PSME3 (Proteasome (prosome, macropain) activator subunit 3 (PA28 gamma; Ki), transc); RALBP1 (RalA-binding protein 1); SSA2 (Sjogren syndrome antigen A2 (60 kDa, ribonucleoprotein autoantigen SS-A/Ro)); and BANK1 (B-cell scaffold protein with ankyrin repeats 1).

In such a scenario a reference to a panel comprising, say, 5, auto-antigens of Table 1 would be a reference to a panel comprising 5 auto-antigens selected from the group consisting of: ZMAT2 (Zinc finger, matrin type 2); BPY2IP1 (MAP1S protein); CEBPG (CCAAT/enhancer binding protein (C/EBP), gamma); E1B-AP5 (E1B-55 kDa-associated protein 5); FUS (Fusion (involved in t(12;16) in malignant liposarcoma)); HAGH (Hydroxyacylglutathione hydrolase); HMG20B (High-mobility group 20B); HOXB6 (Homeo box B6, transcript variant 2); KRT8 (Keratin 8); L1N28 (Lin-28 homolog (C. elegans)); NDUFV3 (NADH dehydrogenase (ubiquinone) flavoprotein 3, 10 kDa); PABPC1 (Poly(A) binding protein, cytoplasmic 1); PHLDA1 (Pleckstrin homology-like domain, family A, member 1); PIAS2 (Msx-interacting-zinc finger, transcript variant alpha); RAB11 FIP3 (RAB11 family interacting protein 3 (class II)); RAN (RAN, member RAS oncogene family); RARA (Retinoic acid receptor alpha); RBMS1 (RNA binding motif, single stranded interacting protein 1, transcript variant); RDBP (RD RNA binding protein); RNF12 (Ring finger protein 12, transcript variant 1); RUFY1 (RUN and FYVE domain containing 1); SMN1 (Survival of motor neuron 1, telomeric); SRPK1 (SFRS protein kinase 1); SSNA1 (Sjogren's syndrome nuclear autoantigen 1); STAU (Staufen, RNA binding protein (Drosophila), transcript variant T3); STK11 (Serine/threonine kinase 11 (Peutz-Jeghers syndrome)); TOM1 (Target of myb1 (chicken)); TXNL2 (Thioredoxin-like, clone MGC:12349); TXNRD1 (Thioredoxin reductase 1, transcript variant 5); ZNF38 (Zinc finger protein 38).

Likewise, in such a scenario, a reference to a panel comprising, say, 5, auto-antigens of Table 2 would be a reference to a panel comprising 5 auto-antigens selected from the group consisting of: ZMAT2 (Zinc finger, matrin type 2); ASPSCR1 (alveolar soft part sarcoma chromosome region, candidate 1); CEBPG (CCAAT/enhancer binding protein (C/EBP), gamma); DDX55 (DEAD (Asp-Glu-Ala-Asp) box polypeptide 55); DOM3Z (dom-3 homolog Z (C. elegans)); E1B-AP5 (E1B-55 kDa-associated protein 5) FUS (Fusion (involved in t(12;16) in malignant liposarcoma)); HMG20B (High-mobility group 20B); KRT8 (Keratin 8); L1N28 Lin-28 homolog (C. elegans)); LNX (ligand of numb-protein X); MAP1S (MAP1S protein); PABPC1 (Poly(A) binding protein, cytoplasmic 1); PHLDA1 (Pleckstrin homology-like domain, family A, member 1); PIAS2 (Msx-interacting-zinc finger, transcript variant alpha); PRKCBP1 (protein kinase C binding protein 1); PRKRA (protein kinase, interferon-inducible double stranded RNA dependent activator); RARA (Retinoic acid receptor, alpha); RDBP (RD RNA binding protein); RPL30 (ribosomal protein L30); RPL31 (ribosomal protein L31); SNK (serum-inducible kinase); SRPK1 (SFRS protein kinase 1); STAU1 (Staufen, RNA binding protein (Drosophila), transcript variant T3); TXNL2 (Thioredoxin-like, clone MGC:12349); VCL (vinculin); ZNF38 (Zinc finger protein 38).

According to one aspect of the present invention therefore there is provided a method of diagnosing a patient as having or being predisposed to developing lupus, said method comprising detecting in a sample taken from said patient one or more auto-antibodies that bind one or more auto-antigens; wherein the presence of said one or more auto-antibodies, or an increase in the concentration of said one or more auto-antibodies, indicates that the patient suffers or is predisposed to develop lupus, wherein said one or more auto-antigens are selected from ZMAT2 (Zinc finger, matrin type 2); BANK1 (B-cell scaffold protein with ankyrin repeats 1); BPY2IP1 (MAP1S protein); CEBPG (CCAAT/enhancer binding protein (C/EBP), gamma); E1B-AP5 (E1B-55 kDa-associated protein 5); FUS (Fusion (involved in t(12;16) in malignant liposarcoma)); HAGH (Hydroxyacylglutathione hydrolase); HMG20B (High-mobility group 20B); HOXB6 (Homeo box B6, transcript variant 2); KRT8 (Keratin 8); L1N28 (Lin-28 homolog (C. elegans)); NDUFV3 (NADH dehydrogenase (ubiquinone) flavoprotein 3, 10 kDa); PABPC1 (Poly(A) binding protein, cytoplasmic 1); PHLDA1 (Pleckstrin homology-like domain, family A, member 1); PIAS2 (Msx-interacting-zinc finger, transcript variant alpha); RAB11 FIP3 (RAB11 family interacting protein 3 (class II)); RAN (RAN, member RAS oncogene family); RARA (Retinoic acid receptor alpha); RBMS1 (RNA binding motif, single stranded interacting protein 1, transcript variant); RDBP (RD RNA binding protein); RNF12 (Ring finger protein 12, transcript variant 1); RUFY1 (RUN and FYVE domain containing 1); SMN1 (Survival of motor neuron 1, telomeric); SRPK1 (SFRS protein kinase 1); SSNA1 (Sjogren's syndrome nuclear autoantigen 1); STAU (Staufen, RNA binding protein (Drosophila), transcript variant T3); STK11 (Serine/threonine kinase 11 (Peutz-Jeghers syndrome)); TOM1 (Target of myb1 (chicken)); TXNL2 (Thioredoxin-like, clone MGC:12349); TXNRD1 (Thioredoxin reductase 1, transcript variant 5); or ZNF38 (Zinc finger protein 38).

Preferably, the method comprises detecting auto-antibodies to one or more of the auto-antigens listed above, in combination with one or more of the auto-antigens IFI16 (Interferon, gamma-inducible protein 16); PSME3 (Proteasome (prosome, macropain) activator subunit 3 (PA28 gamma; Ki), transc), SSA2 (Sjogren syndrome antigen A2 (60 kDa, ribonucleoprotein autoantigen SS-A/Ro)), and RALBP1 (RalA-binding protein 1).

The sample may be a body fluid, and is preferably selected from blood, serum, plasma saliva, lymphatic fluid, wound secretion, urine, faeces, mucus and cerebrospinal fluid (CSF). Typically, the sample may be serum or plasma.

The term “diagnosis” (and for the avoidance of doubt, grammatical variants thereof such as “diagnosing”, “diagnostics” etc.) as used herein is intended to be construed broadly. Thus, for example, the methods and products of the present invention are envisaged as being useful in the following: making a positive or negative diagnosis of lupus in a patient (including subclinical or presymptomatic lupus); assessing the severity of lupus in a patient; assessing an individual's susceptibility to lupus; providing a prognosis of an individual suffering from lupus; monitoring the progression or regression of lupus; monitoring the progression to a flare of the disease or transition from a flare into remission; or monitoring the efficacy of a therapeutic agent to lupus. As indicated above, the methods and products (kits, biomarkers, panels etc.) of the present invention may be used alone to achieve any of the foregoing or may alternatively be combined with one or more other diagnostic/prognostic indicators (e.g. the identification of one or more symptoms characteristic of lupus, the results of one or more biochemical tests etc.). Also, given the difficulties encountered in diagnosing lupus, it should be appreciated that the term “diagnosis” does not necessarily entail an unequivocal diagnosis of lupus in a patient but may rather, for example, provide an indication of the likelihood of lupus being present or absent in an individual.

By a “patient” (the term is used interchangeably herein with the term “individual”) is meant an individual from whom a sample has been obtained e.g. for testing in accordance with a method of the present invention. The term “patient” is not intended to imply that the individual is necessarily undergoing any treatment and thus the term may refer to any individual who is being tested in accordance with a protocol of the present invention.

By “bind” or “bound” used herein is meant a specific interaction strong enough to withstand standard washing procedures in typical protein binding assays.

Accordingly, the present invention comprehends the use of one or more auto-antibodies that bind to said one or more auto-antigens as biomarkers for lupus.

By an increase in the concentration of said one or more auto-antibodies is meant a statistically significant increase in the concentration of the one or more auto-antibodies in said sample taken from the patient as compared with a predetermined mean concentration found in non-diseased, normal patients. A surrogate normal sample can also be used, for example a pooled normal control measured in relation to normal and diseased samples. Typically, the mean concentration of auto-antibodies found in normal patients may be calculated by reference to samples taken from a population of non-diseased individuals or from pooled samples from such individuals. In some embodiments, an increase of more than 5%, more suitably 10%, and suitably more than 15% or 20%, as compared with such mean may be taken as statistically significant. It will be appreciated that the units are arbitrary. In the case of clinical drug evaluation, there will be a change in auto-antibody concentration upon drug treatment; it is anticipated that the levels of most of these auto-antibodies may decrease post drug treatment.

Suitably, said method may comprise the detection of auto-antibodies that bind two or more of said auto-antigens, preferably 3 or more of said auto-antigens, even more preferably 4, 5, 6, 7, 8, 9, 10, 11, 12, 13, 14, 15, 16, 17, 18, 19, 20, 21, 22, 23, 24, 25, 26, 27, 28, 29, 30 or 31 of these auto-antigens. In some embodiments, the patient may be pre-symptomatic.

In another aspect of the present invention there is provided a method of diagnosing a patient as having or being predisposed to developing lupus, said method comprising detecting in a sample from said patient auto-antibodies to a panel comprising 5 or more, preferably 6, 7, 8, 9, 10, 11, 12, 13, 14, 15, 16, 17, 18, 19, 20, 21, 22, 23, 24, 25, 26, 27, 28, 29, 30, 31, 32, 33, 34 or more, even more preferably to a panel comprising all 35, auto-antigens selected from the group consisting of those auto-antigens listed in Table 1 below; wherein the presence or separately a significant increase in the concentration of said auto-antibodies in the sample to substantially all members of the panel indicates that the patient suffers or is predisposed to develop lupus. In some embodiments, said panel may comprise additional auto-antigens (or other antigens) to those specified.

A significant increase in the concentration of the auto-antibodies means a statistically significant increase as determined by methods for statistically analysing experimental results of the kind commonly used in the art. By “substantially all members of the panel” is meant that the sample contains auto-antibodies, or an elevated concentration of auto-antibodies, to at least 50%, preferably at least 75% or 80% and typically at least 90% or 95%, of the auto-antigens included in the panel. It is envisaged that in some embodiments, the presence of auto-antibodies to one or more members of the panel and an increase in the respective concentrations of auto-antibodies to one or more other members of the panel will be indicative of lupus, it being unnecessary in most cases for there to be an increased concentration for all auto-antibodies detected. However, in some cases, the mere presence of auto-antibodies to substantially all members of the panel will be significant.

A significant decrease in the concentration of the auto-antibodies means a statistically significant decrease as determined by methods for statistically analysing experimental results of the kind commonly used in the art. By “substantially all members of the panel” is meant that the sample contains auto-antibodies, or a reduced concentration of auto-antibodies, to at least 50%, preferably at least 75% or 80% and typically at least 90% or 95%, of the auto-antigens included in the panel. It is envisaged that in some embodiments, the presence of auto-antibodies to one or more members of the panel and a decrease in the respective concentrations of auto-antibodies to one or more other members of the panel will be indicative of lupus, it being unnecessary in most cases for there to be a decreased concentration for all auto-antibodies detected.

It is expected that such a panel of biomarkers according to the present invention will provide greatly improved sensitivity or improved specificity in such methods of diagnosis as compared with known biomarkers for lupus.

A panel is defined as a collection of two or more individual markers where the information provided by the panel produces a greater sensitivity and/or specificity than that provided by the markers individually.

As mentioned above, the auto-antigens for lupus are listed in the Tables below (e.g. Table 1). References to gene sequences and uniprot accession numbers for such auto-antigens are also given. However, it will be appreciated that variants of the sequences given, for example allelic variants, orthologues from other mammalian species or other variants (e.g. fragments or antigens with post-translational modifications) that would be bound by the respective auto-antibodies may also be used. Accordingly, it will be understood that the use of such variants are contemplated as being within the scope of the present invention and that references to particular auto-antigens or biomarkers should be construed accordingly. In particular, the present invention comprehends the use of auto-antigens being encoded from respective gene sequences that individually have at least an 80% identity to the corresponding sequences listed in the Tables below (e.g. Table 1). Suitably, such sequences have at least an 85% or 90% identity to the corresponding sequences in the Tables, and preferably they have at least a 95% identity to such sequences, e.g., more than a 96%, 97%, 98% or 99% identity. The skilled person will appreciate that such variants should retain the ability to be bound by the auto-antibodies in a positive patient sample which auto-antibodies recognize the wild-type antigen.

TABLE 1

| Gene symbol | Name | Synonyms | Entrez Gene ID and protein sequence link |
|---|---|---|---|
| ZMAT2 | Zinc finger, | FLJ31121 | 153527; http://ca.expasy.org/uniprot/Q96NC0 |
| | matrin type 2 | | |
| BANK1 | B-cell scaffold | BANK; | 55024; http://ca.expasy.org/uniprot/Q8NDB2 |
| | protein with | FLJ20706; | |
| | ankyrin repeats 1 | FLJ34204 | |
| BPY2IP1 | MAP1S protein | MAP8; C19orf5; | 55201; http://www.ncbi.nlm.nih.gov/entrez/ |
| | | VCY2IP1; | viewer.fcgi?db=protein&val=AAH06358.2 |
| | | FLJ10669; | |
| | | VCY2IP-1; | |
| | | MGC133087 | |
| CEBPG | CCAAT/enhancer | | 1054; http://ca.expasy.org/uniprot/P53567 |
| | binding protein | | |
| | (C/EBP), gamma | | |
| E1B-AP5 | E1B-55 kDa- | E1BAP5; E1B- | 11100; http://ca.expasy.org/uniprot/O76022 |
| | associated | AP5; HNRPUL1; | |
| | protein 5 | FLJ12944 | |
| FUS | Fusion (involved | TLS; CHOP; | 2521; http://ca.expasy.org/uniprot/P35637 |
| | in t(12;16) in | FUS1; FUS- | |
| | malignant | CHOP; | |
| | liposarcoma) | TLS/CHOP; | |
| | | hnRNP-P2 | |
| HAGH | Hydroxyacylglutathione | GLO2; GLX2; | 3029; http://ca.expasy.org/uniprot/Q16775 |
| | hydrolase | GLXII; HAGH1 | |
| HMG20B | High-mobility | SOXL; HMGX2; | 10362; http://ca.expasy.org/uniprot/Q9P0W2 |
| | group 20B | BRAF25; | |
| | | BRAF35; | |
| | | PP7706; pp8857; | |
| | | FLJ26127; | |
| | | SMARCE1r | |
| HOXB6 | Homeo box B6, | HOX2; HU-2; | 3216; http://ca.expasy.org/uniprot/P17509 |
| | transcript variant 2 | HOX2B; Hox-2.2 | |
| IFI16 | Interferon, | PYHIN2; | 3428; http://ca.expasy.org/uniprot/Q16666 |
| | gamma-inducible | IFNGIP1; | |
| | protein 16 | MGC9466 | |
| KRT8 | Keratin 8 | K8; KO; CK8; | 3856; http://ca.expasy.org/uniprot/P05787 |
| | | CYK8; K2C8; | |
| | | CARD2 | |
| LIN28 | Lin-28 homolog | CSDD1; LIN-28; | 79727; http://ca.expasy.org/uniprot/Q9H9Z2 |
| | (C. elegans) | LIN28A; | |
| | | ZCCHC1; | |
| | | FLJ12457 | |
| NDUFV3 | NADH | CI-9KD | 4731; http://ca.expasy.org/uniprot/P56181 |
| | dehydrogenase | | |
| | (ubiquinone) | | |
| | flavoprotein 3, | | |
| | 10 kDa | | |
| PABPC1 | Poly(A) binding | PAB1; PABP; | 26986; http://ca.expasy.org/uniprot/P11940 |
| | protein, | PABP1; | |
| | cytoplasmic 1 | PABPC2; | |
| | | PABPL1 | |
| PHLDA1 | Pleckstrin | PHRIP; TDAG51; | 22822; http://ca.expasy.org/uniprot/Q8WV24 |
| | homology-like | DT1P1B11; | |
| | domain, family A, | MGC131738 | |
| | member 1 | | |
| PIAS2 | Msx-interacting- | miz; MIZ1; SIZ2; | 9063; http://ca.expasy.org/uniprot/Q13105 |
| | zinc finger, | PIASX; ZMIZ4; | |
| | transcript variant | MGC102682; | |
| | alpha | PIASX-BETA; | |
| | | PIASX-ALPHA | |
| PSME3 | Proteasome | Ki; PA28G; REG- | 10197; http://ca.expasy.org/uniprot/Q12920 |
| | (prosome, | GAMMA; PA28- | |
| | macropain) | gamma | |
| | activator subunit | | |
| | 3 (PA28 gamma; | | |
| | Ki), transc | | |
| RAB11FIP3 | RAB11 family | KIAA0665; | 9727; http://ca.expasy.org/uniprot/O75154 |
| | interacting | Rab11-FIP3 | |
| | protein 3 (class | | |
| | II) | | |
| RALBP1 | RalA binding | RIP; RIP1; | 10928; http://ca.expasy.org/uniprot/Q15311 |
| | protein 1 | RLIP76 | |
| RAN | RAN, member | TC4; Gsp1; | 5901; http://ca.expasy.org/uniprot/P17080 |
| | RAS oncogene | ARA24 | |
| | family, | | |
| RARA | Retinoic acid | RAR; NR1B1 | 5914; http://ca.expasy.org/uniprot/P10276 |
| | receptor, alpha | | |
| RBMS1 | RNA binding | YC1; MSSP; | 5937; http://ca.expasy.org/uniprot/P29558 |
| | motif, single | SCR2; MSSP-1; | |
| | stranded | MSSP-2; MSSP- | |
| | interacting | 3; MGC3331; | |
| | protein 1, | MGC15146 | |
| | transcript variant | | |
| RDBP | RD RNA binding | RD; RDP; | 7936; http://ca.expasy.org/uniprot/P18615 |
| | protein | D6S45; NELF-E | |
| RNF12 | Ring finger | RLIM; | 51132; http://ca.expasy.org/uniprot/Q9NVW2 |
| | protein 12, | MGC15161; NY- | |
| | transcript variant 1 | REN-43 | |
| RUFY1 | RUN and FYVE | RABIP4; | 80230; http://ca.expasy.org/uniprot/Q96T51 |
| | domain | ZFYVE12; | |
| | containing 1 | FLJ22251 | |
| SMN1 | Survival of motor | SMA; SMN; | 6606; http://ca.expasy.org/uniprot/Q16637 |
| | neuron 1, | SMA1; SMA2; | |
| | telomeric | SMA3; SMA4; | |
| | | SMA@; SMNT; | |
| | | BCD541; T- | |
| | | BCD541 | |
| SRPK1 | SFRS protein | SFRSK1 | 6732; http://ca.expasy.org/uniprot/Q96SB4 |
| | kinase 1 | | |
| SSA2 | Sjogren | RO60; SSA2; | 6738; http://ca.expasy.org/uniprot/P10155 |
| | syndrome | TROVE2 | |
| | antigen A2 | | |
| | (60 kDa, | | |
| | ribonucleoprotein | | |
| | autoantigen SS- | | |
| | A/Ro) | | |
| SSNA1 | Sjogren's | N14; NA14; NA- | 8636; http://ca.expasy.org/uniprot/O43805 |
| | syndrome | 14 | |
| | nuclear | | |
| | autoantigen 1 | | |
| STAU | Staufen, RNA | STAU; FLJ25010 | 6780; http://ca.expasy.org/uniprot/O95793 |
| | binding protein | | |
| | (Drosophila), | | |
| | transcript variant | | |
| | T3 | | |
| STK11 | Serine/threonine | PJS; LKB1 | 6794; http://ca.expasy.org/uniprot/Q15831 |
| | kinase 11 (Peutz- | | |
| | Jeghers | | |
| | syndrome) | | |
| TOM1 | Target of myb1 | FLJ33404 | 10043; http://ca.expasy.org/uniprot/O60784 |
| | (chicken) | | |
| TXNL2 | Thioredoxin-like, | GRX3; GRX4; | 10539; http://ca.expasy.org/uniprot/O76003 |
| | clone | GLRX4; PICOT; | |
| | MGC: 12349 | TXNL2; TXNL3; | |
| | | FLJ11864; | |
| | | bA500G10.4; | |
| | | GLRX3 | |
| TXNRD1 | Thioredoxin | TR; TR1; TXNR; | 7296; http://ca.expasy.org/uniprot/Q16881 |
| | reductase 1, | TRXR1; GRIM- | |
| | transcript variant 5 | 12; MGC9145 | |
| ZNF38 | Zinc finger | ZNF38; Zipro1; | 7589; http://ca.expasy.org/uniprot/Q9Y5A6 |
| | protein 38 | NY-REN-21; | |
| | | DKFZp434L134; | |
| | | DKFZp686H10254 | |

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleotide base or amino acid occurs in both sequences to yield the number of matched positions, dividing the number of matched positions by the total number of positions in the region of comparison (i.e., the window size), and multiplying the result by 100 to yield the percentage of sequence identity. The identity between two DNA or protein sequences may be determined using computer programs known in the art, such as GAP software provided in the GCG program package with default parameters (Needleman and Wunsch 1970 J Mol Biol 48: 443-453). The identity between two protein sequences is also displayed after searching a protein database with Blast2 (Altschul et al (1997) Nucleic Acids Res. 25 (17) 3389-3402).

It is anticipated that the methods of the present invention may provide greater sensitivity and/or specificity for the diagnosis of lupus than previously available in the art.

By “sensitivity” is meant the proportion of persons with the disease phenotype who test positive.

By “specificity” is meant the proportion of persons without the disease phenotype who test negative.

Advantageously, the methods of the invention may have a sensitivity of >67%, preferably >70%, even more preferably >75%, 80%, 85%, 90%, and most preferably >95%, or a specificity of >85%, preferably >90%, even more preferably >95%, and most preferably >98%.

According to yet another aspect of the invention there is provided a method of monitoring the progression or regression of lupus in a patient, said method comprising detecting in a first sample obtained from a patient one or more auto-antibodies that bind one or more auto-antigens selected from the group consisting of ZMAT2 (Zinc finger, matrin type 2); BANK1 (B-cell scaffold protein with ankyrin repeats 1); BPY2IP1 (MAP1S protein); CEBPG (CCAAT/enhancer binding protein (C/EBP), gamma); E1B-AP5 (E1B-55 kDa-associated protein 5); FUS (Fusion (involved in t(12;16) in malignant liposarcoma)); HAGH (Hydroxyacylglutathione hydrolase); HMG20B (High-mobility group 20B); HOXB6 (Homeo box B6, transcript variant 2); KRT8 (Keratin 8); L1N28 (Lin-28 homolog (C. elegans)); NDUFV3 (NADH dehydrogenase (ubiquinone) flavoprotein 3, 10 kDa); PABPC1 (Poly(A) binding protein, cytoplasmic 1); PHLDA1 (Pleckstrin homology-like domain, family A, member 1); PIAS2 (Msx-interacting-zinc finger, transcript variant alpha); RAB11FIP3 (RAB11 family interacting protein 3 (class II)); RAN (RAN, member RAS oncogene family); RARA (Retinoic acid receptor alpha); RBMS1 (RNA binding motif, single stranded interacting protein 1, transcript variant); RDBP (RD RNA binding protein); RNF12 (Ring finger protein 12, transcript variant 1); RUFY1 (RUN and FYVE domain containing 1); SMN1 (Survival of motor neuron 1, telomeric); SRPK1 (SFRS protein kinase 1); SSNA1 (Sjogren's syndrome nuclear autoantigen 1); STAU (Staufen, RNA binding protein (Drosophila), transcript variant T3); STK11 (Serine/threonine kinase 11 (Peutz-Jeghers syndrome)); TOM1 (Target of myb1 (chicken)); TXNL2 (Thioredoxin-like, clone MGC:12349); TXNRD1 (Thioredoxin reductase 1, transcript variant 5); or ZNF38 (Zinc finger protein 38), or auto-antibodies that bind to a panel comprising 5 or more, preferably 6, 7, 8, 9, 10, 11, 12, 13, 14, 15, 16, 17, 18, 19, 20, 21, 22, 23, 24, 25, 26, 27, 28, 29, 30, 31, 32, 33, 34 or more, even more preferably to a panel comprising all 35, auto-antigens selected from the group consisting of those auto-antigens listed in Table 1; detecting said auto-antibodies in a second sample obtained from the patient at a time later than the first sample; wherein a change in the auto-antibodies detected in the first and second samples is indicative of the progression or regression of lupus.

According to yet another aspect of the invention there is provided a method of monitoring the progression towards a flare of the disease, or the transition from a flare to remission, of lupus in a patient, said method comprising detecting in a first sample obtained from a patient one or more auto-antibodies that bind one or more auto-antigens selected from the group consisting of ZMAT2 (Zinc finger, matrin type 2); BANK1 (B-cell scaffold protein with ankyrin repeats 1); BPY2IP1 (MAP1S protein); CEBPG (CCAAT/enhancer binding protein (C/EBP), gamma); E1B-AP5 (E1B-55 kDa-associated protein 5); FUS (Fusion (involved in t(12;16) in malignant liposarcoma)); HAGH (Hydroxyacylglutathione hydrolase); HMG20B (High-mobility group 20B); HOXB6 (Homeo box B6, transcript variant 2); KRT8 (Keratin 8); L1N28 (Lin-28 homolog (C. elegans)); NDUFV3 (NADH dehydrogenase (ubiquinone) flavoprotein 3, 10 kDa); PABPC1 (Poly(A) binding protein, cytoplasmic 1); PHLDA1 (Pleckstrin homology-like domain, family A, member 1); PIAS2 (Msx-interacting-zinc finger, transcript variant alpha); RAB11FIP3 (RAB11 family interacting protein 3 (class II)); RAN (RAN, member RAS oncogene family); RARA (Retinoic acid receptor alpha); RBMS1 (RNA binding motif, single stranded interacting protein 1, transcript variant); RDBP (RD RNA binding protein); RNF12 (Ring finger protein 12, transcript variant 1); RUFY1 (RUN and FYVE domain containing 1); SMN1 (Survival of motor neuron 1, telomeric); SRPK1 (SFRS protein kinase 1); SSNA1 (Sjogren's syndrome nuclear autoantigen 1); STAU (Staufen, RNA binding protein (Drosophila), transcript variant T3); STK1_(Serine/threonine kinase 11 (Peutz-Jeghers syndrome)); TOM1 (Target of myb1 (chicken)); TXNL2 (Thioredoxin-like, clone MGC:12349); TXNRD1 (Thioredoxin reductase 1, transcript variant 5); or ZNF38 (Zinc finger protein 38), or auto-antibodies that bind to a panel comprising 5 or more, preferably 6, 7, 8, 9, 10, 11, 12, 13, 14, 15, 16, 17, 18, 19, 20, 21, 22, 23, 24, 25, 26, 27, 28, 29, 30, 31, 32, 33, 34 or more, even more preferably to a panel comprising all 35, auto-antigens selected from the group consisting of those auto-antigens listed in Table 1; detecting said auto-antibodies in a second sample obtained from the patient at a time later than the first sample; wherein a change in the auto-antibodies detected in the first and second samples is indicative of the progression towards a flare, or transition from a flare to a period of remission of the disease.

A change in the auto-antibodies may be a change in the number of different auto-antigens to which auto-antibodies are detected in the first as compared to the second sample, or it may be a change in the concentration of an auto-antibody in the first as compared to the second sample, or it may be a combination of both.

Whilst the biomarkers specifically disclosed herein in relation to the present invention are elevated in lupus patients (and therefore an increased auto-antibody concentration of the biomarkers are indicative of lupus), auto-antibodies for which a decrease in concentration indicates that a patient suffers from or is presymptomatic or predisposed to develop lupus are also envisaged, and may also provide useful biomarkers for lupus. Such markers may be used in combination with the methods or products of the present invention so that an increase in certain biomarkers is indicative of lupus whilst a decrease in certain other biomarkers is also indicative of lupus.

According to yet another aspect of the present invention there is provided a method of monitoring the efficacy of a therapeutic agent to lupus, comprising detecting in a first sample obtained from a patient the presence of one or more auto-antibodies that bind one or more auto-antigens selected from the group consisting of ZMAT2 (Zinc finger, matrin type 2); BANK1 (B-cell scaffold protein with ankyrin repeats 1); BPY2IP1 (MAP1S protein); CEBPG (CCAAT/enhancer binding protein (C/EBP), gamma); E1B-AP5 (E1B-55 kDa-associated protein 5); FUS (Fusion (involved in t(12;16) in malignant liposarcoma)); HAGH (Hydroxyacylglutathione hydrolase); HMG20B (High-mobility group 20B); HOXB6 (Homeo box B6, transcript variant 2); KRT8 (Keratin 8); L1N28 (Lin-28 homolog (C. elegans)); NDUFV3 (NADH dehydrogenase (ubiquinone) flavoprotein 3, 10 kDa); PABPC1 (Poly(A) binding protein, cytoplasmic 1); PHLDA1 (Pleckstrin homology-like domain, family A, member 1); PIAS2 (Msx-interacting-zinc finger, transcript variant alpha); RAB11FIP3 (RAB11 family interacting protein 3 (class II)); RAN (RAN, member RAS oncogene family); RARA (Retinoic acid receptor alpha); RBMS1 (RNA binding motif, single stranded interacting protein 1, transcript variant); RDBP (RD RNA binding protein); RNF12 (Ring finger protein 12, transcript variant 1); RUFY1 (RUN and FYVE domain containing 1); SMN1 (Survival of motor neuron 1, telomeric); SRPK1 (SFRS protein kinase 1); SSNA1 (Sjogren's syndrome nuclear autoantigen 1); STAU (Staufen, RNA binding protein (Drosophila), transcript variant T3); STK11_(Serine/threonine kinase 11 (Peutz-Jeghers syndrome)); TOM1 (Target of myb1 (chicken)); TXNL2 (Thioredoxin-like, clone MGC:12349); TXNRD1 (Thioredoxin reductase 1, transcript variant 5); or ZNF38 (Zinc finger protein 38), or auto-antibodies that bind to a panel comprising 5 or more, preferably 6, 7, 8, 9, 10, 11, 12, 13, 14, 15, 16, 17, 18, 19, 20, 21, 22, 23, 24, 25, 26, 27, 28, 29, 30, 31, 32, 33, 34 or more, even more preferably to a panel comprising all 35, auto-antigens selected from the group consisting of those auto-antigens listed in Table 1; administering the therapeutic agent to the patient; detecting said one or more auto-antibodies in a second sample taken from the patient taken after the administration of the therapeutic agent; and comparing the one or more auto-antibodies in said first and second samples respectively, wherein a change in the auto-antibodies in the first and second sample is indicative of the efficacy of the therapeutic agent to lupus.

The first sample may be taken prior to the first administration of a therapeutic agent, or after treatment has already commenced. The method can be used to monitor the efficacy of treatment over time, e.g., over days, weeks, months or years.

The therapeutic agent may be any therapeutic agent that can be beneficial for the treatment of lupus, for example anti-rheumatic agents or anti-inflammatory agents.

According to yet another aspect of the present invention, there is provided a method of predicting whether a patient suffering from lupus will respond to treatment with a therapeutic agent, comprising detecting the presence or absence of one or more auto-antibodies that bind to one or more auto-antigens selected from the group consisting of ZMAT2 (Zinc finger, matrin type 2); BANK1 (B-cell scaffold protein with ankyrin repeats 1); BPY2IP1 (MAP1S protein); CEBPG (CCAAT/enhancer binding protein (C/EBP), gamma); E1B-AP5 (E1B-55 kDa-associated protein 5); FUS (Fusion (involved in t(12;16) in malignant liposarcoma)); HAGH (Hydroxyacylglutathione hydrolase); HMG20B (High-mobility group 20B); HOXB6 (Homeo box B6, transcript variant 2); KRT8 (Keratin 8); L1N28 (Lin-28 homolog (C. elegans)); NDUFV3 (NADH dehydrogenase (ubiquinone) flavoprotein 3, 10 kDa); PABPC1 (Poly(A) binding protein, cytoplasmic 1); PHLDA1 (Pleckstrin homology-like domain, family A, member 1); PIAS2 (Msx-interacting-zinc finger, transcript variant alpha); RAB11FIP3 (RAB11 family interacting protein 3 (class II)); RAN (RAN, member RAS oncogene family); RARA (Retinoic acid receptor alpha); RBMS1 (RNA binding motif, single stranded interacting protein 1, transcript variant); RDBP (RD RNA binding protein); RNF12 (Ring finger protein 12, transcript variant 1); RUFY1 (RUN and FYVE domain containing 1); SMN1 (Survival of motor neuron 1, telomeric); SRPK1 (SFRS protein kinase 1); SSNA1 (Sjogren's syndrome nuclear autoantigen 1); STAU (Staufen, RNA binding protein (Drosophila), transcript variant T3); STK11_(Serine/threonine kinase 11 (Peutz-Jeghers syndrome)); TOM1 (Target of myb1 (chicken)); TXNL2 (Thioredoxin-like, clone MGC:12349); TXNRD1 (Thioredoxin reductase 1, transcript variant 5); or ZNF38 (Zinc finger protein 38), or auto-antibodies that bind to a panel comprising 5 or more, preferably 6, 7, 8, 9, 10, 11, 12, 13, 14, 15, 16, 17, 18, 19, 20, 21, 22, 23, 24, 25, 26, 27, 28, 29, 30, 31, 32, 33, 34 or more, even more preferably to a panel comprising all 35, auto-antigens selected from the group consisting of those auto-antigens listed in Table 1; wherein the presence or absence of said one or more auto-antibodies indicates that the patient will or will not respond to treatment with said therapeutic agent.

The auto-antibodies may be detected by any means known in the art. This includes the use of protein arrays, bead-based assays, Western blots, immunoprecipitation, silver staining, dot blots, etc. The binding of the auto-antibodies can be detected by any means, including enzyme-linked assays such as ELISAs, radioimmunoassays (RIAs), DELFIA assays, fluorescence-based detection methods, bead assays such as Luminex bead assays, etc.

In some embodiments, arrays of said auto-antigens may be employed for detecting said auto-antibodies, for example arrays prepared according to the methods disclosed by WO-A-01/57198 or WO-A-2003/064656; preferred detection methods are fluorescence-based detection methods.

The patient may have or be predisposed to develop the disease naturally. However, a patient may also develop lupus as a result of environmental factors, for example as a side effect of treatment with therapeutic agents or as a result of exposure to toxic materials, or as a result of manipulation, e.g., in animal models used in pharmaceutical research and development. The patient may be a human being, but can also be a non-human animal such, for example, as a mouse, rat, guinea pig, cat, dog, horse, pig, cow, non-human primate (e.g. monkey), or any other animal used for research and drug testing.

Each of the aspects and features of the invention and methods as described above are applicable to the use of auto-antigens from Tables 1, 2, 3, 4 and 5.

Therefore according to the present invention there is provided a method of diagnosing a patient as having or being predisposed to developing lupus, said method comprising detecting in a sample taken from said patient one or more auto-antibodies that bind one or more auto-antigens; wherein the presence of said one or more auto-antibodies, or an increase in the concentration of said one or more auto-antibodies, indicates that the patient suffers from or is predisposed to develop lupus, wherein said one or more antigens are selected from the auto-antigens set out in Table 2.

TABLE 2

| Gene | | | Entrez Gene ID and |
|---|---|---|---|
| symbol | Name | Synonyms | protein sequence link |
| ZMAT2 | Zinc finger, matrin type 2 | FLJ31121 | 153527; |
| | | | http://ca.expasy.org/uniprot/Q96NC0 |
| ASPSCR1 | alveolar soft part | TUG; ASPL; ASPS; RCC17; | 79058; |
| | sarcoma chromosome | UBXD9; UBXN9; ASPCR1; | http://ca.expasy.org/uniprot/Q7Z6N7 |
| | region, candidate 1 | FLJ45380; ASPSCR1 | |
| BANK1 | B-cell scaffold protein | BANK; FLJ20706; FLJ34204 | 55024; |
| | with ankyrin repeats 1 | | http://ca.expasy.org/uniprot/Q8NDB2 |
| CEBPG | CCAAT/enhancer | GPE1BP; IG/EBP-1; CEBPG | 1054; |
| | binding protein | | http://ca.expasy.org/uniprot/P53567 |
| | (C/EBP), gamma | | |
| DDX55 | DEAD (Asp-Glu-Ala- | FLJ16577; KIAA1595; | 57696; |
| | Asp) box polypeptide | MGC33209; DDX55 | http://ca.expasy.org/uniprot/Q8NHQ9 |
| | 55 | | |
| DOM3Z | dom-3 homolog Z (C. elegans) | NG6; DOM3L; DOM3Z | 1797; |
| | | | http://ca.expasy.org/uniprot/O77932 |
| E1B-AP5 | E1B-55 kDa-associated | E1BAP5; E1B-AP5; | 11100; |
| | protein 5 | HNRPUL1; FLJ12944 | http://ca.expasy.org/uniprot/O76022 |
| FUS | Fusion (involved in | TLS; CHOP; FUS1; FUS- | 2521; |
| | t(12;16) in malignant | CHOP; TLS/CHOP; hnRNP- | http://ca.expasy.org/uniprot/P35637 |
| | liposarcoma) | P2 | |
| HMG20B | High-mobility group | SOXL; HMGX2; BRAF25; | 10362; |
| | 20B | BRAF35; PP7706; pp8857; | http://ca.expasy.org/uniprot/Q9P0W2 |
| | | FLJ26127; SMARCE1r | |
| IFI16 | Interferon, gamma- | PYHIN2; IFNGIP1; MGC9466 | 3428; |
| | inducible protein 16 | | http://ca.expasy.org/uniprot/Q16666 |
| KRT8 | Keratin 8 | K8; KO; CK8; CYK8; K2C8; | 3856; |
| | | CARD2 | http://ca.expasy.org/uniprot/P05787 |
| LIN28 | Lin-28 homolog (C. elegans) | CSDD1; LIN-28; LIN28A; | 79727; |
| | | ZCCHC1; FLJ12457 | http://ca.expasy.org/uniprot/Q9H9Z2 |
| LNX | ligand of numb-protein X | LNX; MPDZ; PDZRN2; LNX1 | 84708; |
| | | | http://ca.expasy.org/uniprot/Q8TBB1 |
| MAP1S | MAP1S protein | MAP8; C19orf5; VCY2IP1; | 55201; |
| | | FLJ10669; VCY2IP-1; | http://ca.expasy.org/uniprot/Q66K74 |
| | | MGC133087 | |
| PABPC1 | Poly(A) binding protein, | PAB1; PABP; PABP1; | 26986; |
| | cytoplasmic 1 | PABPC2; PABPL1 | http://ca.expasy.org/uniprot/P11940 |
| PHLDA1 | Pleckstrin homology- | PHRIP; TDAG51; DT1P1B11; | 22822; |
| | like domain, family A, | MGC131738 | http://ca.expasy.org/uniprot/Q8WV24 |
| | member 1 | | |
| PIAS2 | Msx-interacting-zinc | miz; MIZ1; SIZ2; PIASX; | 9063; |
| | finger, transcript variant | ZMIZ4; MGC102682; PIASX- | http://ca.expasy.org/uniprot/Q13105 |
| | alpha | BETA; PIASX-ALPHA | |
| PRKCBP1 | protein kinase C | RACK7; PRKCBP1; | 23613; |
| | binding protein 1 | PRO2893; MGC31836; | http://ca.expasy.org/uniprot/Q9H2G5 |
| | | ZMYND8 | |
| PRKRA | protein kinase, | RAX; PACT; DYT16; HSD14; | 8575; |
| | interferon-inducible | PRKRA | http://ca.expasy.org/uniprot/O75569 |
| | double stranded RNA | | |
| | dependent activator | | |
| PSME3 | Proteasome (prosome, | Ki; PA28G; REG-GAMMA; | 10197; |
| | macropain) activator | PA28-gamma | http://ca.expasy.org/uniprot/Q12920 |
| | subunit 3 (PA28 | | |
| | gamma; Ki) | | |
| RALBP1 | RalA binding protein 1 | RIP; RIP1; RLIP76 | 10928; |
| | | | http://ca.expasy.org/uniprot/Q15311 |
| RARA | Retinoic acid receptor, | RAR; NR1B1 | 5914; |
| | alpha | | http://ca.expasy.org/uniprot/P10276 |
| RDBP | RD RNA binding | RD; RDP; D6S45; NELF-E | 7936; |
| | protein | | http://ca.expasy.org/uniprot/P18615 |
| RPL30 | ribosomal protein L30 | RPL30 | 6156; |
| | | | http://ca.expasy.org/uniprot/P04645 |
| RPL31 | ribosomal protein L31 | MGC88191; RPL31 | 6160; |
| | | | http://ca.expasy.org/uniprot/P62899 |
| SNK | serum-inducible kinase | SNK; PLK2 | 10769; |
| | | | http://ca.expasy.org/uniprot/Q9NYY3 |
| SRPK1 | SFRS protein kinase 1 | SFRSK1 | 6732; |
| | | | http://ca.expasy.org/uniprot/Q96SB4 |
| SSA2 | Sjogren syndrome | RO60; SSA2; TROVE2 | 6738; |
| | antigen A2 (60 kDa, | | http://ca.expasy.org/uniprot/P10155 |
| | ribonucleoprotein | | |
| | autoantigen SS-A/Ro) | | |
| STAU1 | Staufen, RNA binding | STAU; FLJ25010 | 6780; |
| | protein (Drosophila), | | http://ca.expasy.org/uniprot/O95793 |
| | transcript variant T3 | | |
| TXNL2 | Thioredoxin-like, clone | GRX3; GRX4; GLRX4; | 10539; |
| | MGC: 12349 | PICOT; TXNL2; TXNL3; | http://ca.expasy.org/uniprot/O76003 |
| | | FLJ11864; bA500G10.4; | |
| | | GLRX3 | |
| VCL | vinculin | MVCL; CMD1W; VCL | 7414; |
| | | | http://ca.expasy.org/uniprot/P18206 |
| ZNF38 | Zinc finger protein 38 | ZNF38; Zipro1; NY-REN-21; | 7589; |
| | | DKFZp434L134; | http://ca.expasy.org/uniprot/Q9Y5A6 |
| | | DKFZp686H10254 | |

In another aspect of the present invention provides a method of diagnosing a patient as having or being predisposed to developing lupus, said method comprising detecting in a sample from said patient auto-antibodies to a panel comprising 5 or more, preferably 6, 7, 8, 9, 10, 11, 12, 13, 14, 15, 16, 17, 18, 19, 20, 21, 22, 23, 24, 25, 26, 27, 28, 29, 30, 31, or more, even more preferably to a panel comprising all 32 auto-antigens selected from the group of auto-antigens listed in Table 2.

According to yet another aspect of the invention there is provided a method of monitoring the progression or regression of lupus in a patient, said method comprising detecting in a first sample obtained from a patient one or more auto-antibodies that bind one or more auto-antigens selected from the group of auto-antigens set out in Table 2, or auto-antigens that bind to a panel comprising 5 or more, preferably, 6, 7, 8, 9, 10, 11, 12, 13, 14, 15, 16, 17, 18, 19, 20, 21, 22, 23, 24, 25, 26, 27, 28, 29, 30, 31, or more or even more preferably to a panel comprising all 32, auto-antigens selected from the group of auto-antigens listed in Table 2.

According to yet another aspect of the present invention there is provided a method of monitoring the efficacy of the therapeutic agent lupus, comprising detecting in a first sample obtained from a patient the presence of one or more auto-antibodies that bind to one or more auto-antigens selected from the group consisting of the auto-antigens in Table 2 or to antigens that bind to a panel comprising 5 or more, preferably 6, 7, 8, 9, 10, 11, 12, 13, 14, 15, 16, 17, 18, 19, 20, 21, 22, 23, 24, 25, 26, 27, 28, 29, 30, 31 or more or even more preferably to a panel comprising all 32, auto-antigens selected from the group of auto-antigens listed in Table 2.

According to yet another aspect of the present invention there is provided a method of predicting whether a patient suffering from lupus will respond to treatment with a therapeutic agent comprising detecting the presence or absence of one or more auto-antibodies that bind to one or more auto-antigens selected from the group consisting of the auto-antigens set out in Table 2, or auto-antibodies that bind to a panel comprising 5 or more, preferably 6, 7, 8, 9, 10, 11, 12, 13, 14, 15, 16, 17, 18, 19, 20, 21, 22, 23, 24, 25, 26, 27, 28, 29, 30, 31, or more, or preferably to a panel comprising all 32 auto-antigens selected from those auto-antigens listed in Table 2.

As explained above the combined sets of 35 and 32 biomarkers produces 45 biomarkers which have been found to be useful in detecting lupus. These biomarkers are set out in Table 3 below.

TABLE 3

| Gene | | | Entrez Gene ID and |
|---|---|---|---|
| symbol | Name | Synonyms | protein sequence link |
| ZMAT2 | Zinc finger, matrin type 2 | FLJ31121 | 153527; |
| | | | http://ca.expasy.org/uniprot/Q96NC0 |
| ASPSCR1 | alveolar soft part sarcoma | TUG; ASPL; ASPS; RCC17; | 79058; |
| | chromosome region, | UBXD9; UBXN9; ASPCR1; | http://ca.expasy.org/uniprot/Q7Z6N7 |
| | candidate 1 | FLJ45380; ASPSCR1 | |
| BANK1 | B-cell scaffold protein with | BANK; FLJ20706; FLJ34204 | 55024; |
| | ankyrin repeats 1 | | http://ca.expasy.org/uniprot/Q8NDB2 |
| BPY2IP1 | MAP1S protein | MAP8; C19orf5; VCY2IP1; | 55201; |
| | | FLJ10669; VCY2IP-1; | http://ca.expasy.org/uniprot/Q66K74 |
| | | MGC133087 | |
| CEBPG | CCAAT/enhancer binding | GPE1BP; IG/EBP-1; CEBPG | 1054; |
| | protein (C/EBP), gamma | | http://ca.expasy.org/uniprot/P53567 |
| DDX55 | DEAD (Asp-Glu-Ala-Asp) | FLJ16577; KIAA1595; | 57696; |
| | box polypeptide 55 | MGC33209; DDX55 | http://ca.expasy.org/uniprot/Q8NHQ9 |
| DOM3Z | dom-3 homolog Z (C. elegans) | NG6; DOM3L; DOM3Z | 1797; |
| | | | http://ca.expasy.org/uniprot/O77932 |
| E1B-AP5 | E1B-55 kDa-associated | E1BAP5; E1B-AP5; | 11100; |
| | protein 5 | HNRPUL1; FLJ12944 | http://ca.expasy.org/uniprot/O76022 |
| FUS | Fusion (involved in | TLS; CHOP; FUS1; FUS- | 2521; |
| | t(12;16) in malignant | CHOP; TLS/CHOP; hnRNP- | http://ca.expasy.org/uniprot/P35637 |
| | liposarcoma) | P2 | |
| HAGH | Hydroxyacylglutathione | GLO2; GLX2; GLXII; HAGH1 | 3029; |
| | hydrolase | | http://ca.expasy.org/uniprot/Q16775 |
| HMG20B | High-mobility group 20B | SOXL; HMGX2; BRAF25; | 10362; |
| | | BRAF35; PP7706; pp8857; | http://ca.expasy.org/uniprot/Q9P0W2 |
| | | FLJ26127; SMARCE1r | |
| HOXB6 | Homeo box B6, transcript | HOX2; HU-2; HOX2B; Hox- | 3216; |
| | variant 2 | 2.2 | http://ca.expasy.org/uniprot/P17509 |
| IFI16 | Interferon, gamma- | PYHIN2; IFNGIP1; MGC9466 | 3428; |
| | inducible protein 16 | | http://ca.expasy.org/uniprot/Q16666 |
| KRT8 | Keratin 8 | K8; KO; CK8; CYK8; K2C8; | 3856; |
| | | CARD2 | http://ca.expasy.org/uniprot/P05787 |
| LIN28 | Lin-28 homolog (C. elegans) | CSDD1; LIN-28; LIN28A; | 79727; |
| | | ZCCHC1; FLJ12457 | http://ca.expasy.org/uniprot/Q9H9Z2 |
| LNX | ligand of numb-protein X | LNX; MPDZ; PDZRN2; LNX1 | 84708; |
| | | | http://ca.expasy.org/uniprot/Q8TBB1 |
| NDUFV3 | NADH dehydrogenase | CI-9KD | 4731; |
| | (ubiquinone) flavoprotein | | http://ca.expasy.org/uniprot/P56181 |
| | 3, 10 kDa | | |
| PABPC1 | Poly(A) binding protein, | PAB1; PABP; PABP1; | 26986; |
| | cytoplasmic 1 | PABPC2; PABPL1 | http://ca.expasy.org/uniprot/P11940 |
| PHLDA1 | Pleckstrin homology-like | PHRIP; TDAG51; DT1P1B11; | 22822; |
| | domain, family A, member 1 | MGC131738 | http://ca.expasy.org/uniprot/Q8WV24 |
| PIAS2 | Msx-interacting-zinc | miz; MIZ1; SIZ2; PIASX; | 9063; |
| | finger, transcript variant | ZMIZ4; MGC102682; PIASX- | http://ca.expasy.org/uniprot/Q13105 |
| | alpha | BETA; PIASX-ALPHA | |
| PRKCBP1 | protein kinase C binding | RACK7; PRKCBP1; | 23613; |
| | protein 1 | PRO2893; MGC31836; | http://ca.expasy.org/uniprot/Q9H2G5 |
| | | ZMYND8 | |
| PRKRA | protein kinase, interferon- | RAX; PACT; DYT16; HSD14; | 8575; |
| | inducible double stranded | PRKRA | http://ca.expasy.org/uniprot/O75569 |
| | RNA dependent activator | | |
| PSME3 | Proteasome (prosome, | Ki; PA28G; REG-GAMMA; | 10197; |
| | macropain) activator | PA28-gamma | http://ca.expasy.org/uniprot/Q12920 |
| | subunit 3 (PA28 gamma; | | |
| | Ki) | | |
| RAB11FIP3 | RAB11 family interacting | KIAA0665; Rab11-FIP3 | 9727; |
| | protein 3 (class II) | | http://ca.expasy.org/uniprot/O75154 |
| RALBP1 | RalA binding protein 1 | RIP; RIP1; RLIP76 | 10928; |
| | | | http://ca.expasy.org/uniprot/Q15311 |
| RAN | RAN, member RAS | TC4; Gsp1; ARA24 | 5901; |
| | oncogene family | | http://ca.expasy.org/uniprot/P17080 |
| RARA | Retinoic acid receptor, | RAR; NR1B1 | 5914; |
| | alpha | | http://ca.expasy.org/uniprot/P10276 |
| RBMS1 | RNA binding motif, single | YC1; MSSP; SCR2; MSSP-1; | 5937; |
| | stranded interacting | MSSP-2; MSSP-3; | http://ca.expasy.org/uniprot/P29558 |
| | protein 1, transcript | MGC3331; MGC15146 | |
| | variant | | |
| RDBP | RD RNA binding protein | RD; RDP; D6S45; NELF-E | 7936; |
| | | | http://ca.expasy.org/uniprot/P18615 |
| RNF12 | Ring finger protein 12, | RLIM; MGC15161; NY-REN- | 51132; |
| | transcript variant 1 | 43 | http://ca.expasy.org/uniprot/Q9NVW2 |
| RPL30 | ribosomal protein L30 | RPL30 | 6156; |
| | | | http://ca.expasy.org/uniprot/P04645 |
| RPL31 | ribosomal protein L31 | MGC88191; RPL31 | 6160; |
| | | | http://ca.expasy.org/uniprot/P62899 |
| RUFY1 | RUN and FYVE domain | RABIP4; ZFYVE12; | 80230; |
| | containing 1 | FLJ22251 | http://ca.expasy.org/uniprot/Q96T51 |
| SMN1 | Survival of motor neuron | SMA; SMN; SMA1; SMA2; | 6606; |
| | 1, telomeric | SMA3; SMA4; SMA@; | http://ca.expasy.org/uniprot/Q16637 |
| | | SMNT; BCD541; T-BCD541 | |
| SNK | serum-inducible kinase | SNK; PLK2 | 10769; |
| | | | http://ca.expasy.org/uniprot/Q9NYY3 |
| SRPK1 | SFRS protein kinase 1 | SFRSK1 | 6732; |
| | | | http://ca.expasy.org/uniprot/Q96SB4 |
| SSA2 | Sjogren syndrome | RO60; SSA2; TROVE2 | 6738; |
| | antigen A2 (60 kDa, | | http://ca.expasy.org/uniprot/P10155 |
| | ribonucleoprotein | | |
| | autoantigen SS-A/Ro) | | |
| SSNA1 | Sjogren's syndrome | N14; NA14; NA-14 | 8636; |
| | nuclear autoantigen 1 | | http://ca.expasy.org/uniprot/O43805 |
| STAU | Staufen, RNA binding | STAU; FLJ25010 | 6780; |
| | protein (Drosophila), | | http://ca.expasy.org/uniprot/O95793 |
| | transcript variant T3 | | |
| STK11 | Serine/threonine kinase | PJS; LKB1 | 6794; |
| | 11 (Peutz-Jeghers | | http://ca.expasy.org/uniprot/Q15831 |
| | syndrome) | | |
| TOM1 | Target of myb1 (chicken) | FLJ33404 | 10043; |
| | | | http://ca.expasy.org/uniprot/O60784 |
| TXNL2 | Thioredoxin-like, clone | GRX3; GRX4; GLRX4; | 10539; |
| | MGC: 12349 | PICOT; TXNL2; TXNL3; | http://ca.expasy.org/uniprot/O76003 |
| | | FLJ11864; bA500G10.4; | |
| | | GLRX3 | |
| TXNRD1 | Thioredoxin reductase 1, | TR; TR1; TXNR; TRXR1; | 7296; |
| | transcript variant 5 | GRIM-12; MGC9145 | http://ca.expasy.org/uniprot/Q16881 |
| VCL | vinculin | MVCL; CMD1W; VCL | 7414; |
| | | | http://ca.expasy.org/uniprot/P18206 |
| ZNF38 | Zinc finger protein 38 | ZNF38; Zipro1; NY-REN-21; | 7589; |
| | | DKFZp434L134; | http://ca.expasy.org/uniprot/Q9Y5A6 |
| | | DKFZp686H10254 | |

Therefore according to the present invention there is provided a method of diagnosing a patient as having or being predisposed to developing lupus, said method comprising detecting in a sample taken from said patient one or more auto-antibodies that bind one or more auto-antigens; wherein the presence of said one or more auto-antibodies, or an increase in the concentration of said one or more auto-antibodies, indicates that the patient suffers or is predisposed to develop lupus, wherein said one or more antigens are selected from the auto-antigens set out in Table 3.

In another aspect of the present invention there is provided a method of diagnosing a patient as having or being predisposed to developing lupus, said method comprising detecting in a sample from said patient auto-antibodies to a panel comprising 5 or more, preferably 6, 7, 8, 9, 10, 11, 12, 13, 14, 15, 16, 17, 18, 19, 20, 21, 22, 23, 24, 25, 26, 27, 28, 29, 30, 31, 33, 34, 35, 36, 37, 38, 39, 40, 41, 42, 43, 44, or more, even more preferably to a panel comprising all 45 auto-antigens selected from the group consisting of those auto-antigens listed in Table 3.

According to yet another aspect of the invention there is provided a method of monitoring the progression or regression of lupus in a patient, said method comprising detecting in a first sample obtained from a patient one or more auto-antibodies that bind one or more auto-antigens selected from the group of auto-antigens set out in Table 2, or auto-antigens that bind to a panel comprising 5 or more, preferably, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 or more, auto-antigens selected from the auto-antigens listed in Table 3.

According to yet another aspect of the present invention there is provided a method of monitoring the efficacy of the therapeutic agent lupus, comprising detecting in a first sample obtained from a patient the presence of one or more auto-antibodies that bind to one or more auto-antigens selected from the group consisting of the auto-antigens in Table 3 or to antigens that bind to a panel comprising 5 or more, preferably 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or more or even more preferably to a panel comprising all 45 auto-antigens selected from the auto-antigens listed in Table 3.

According to yet another aspect of the present invention there is provided a method of predicting whether a patient suffering from lupus will respond to treatment with a therapeutic agent comprising detecting the presence or absence of one or more auto-antibodies that bind to one or more auto-antigens selected from the group consisting of the auto-antigens set out in Table 2, or auto-antibodies that bind to a panel comprising 5 or more, preferably 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44 or more or preferably to a panel comprising all 45 auto-antigens selected from those auto-antigens listed in Table 3.

Comparison of the set 35 biomarkers in Table 1 with the set of 32 biomarkers in Table 2 reveals a group of shared biomarkers or biomarkers in common.

The shared biomarkers are set out in Table 4, whilst the unshared biomarkers, or those biomarkers unique to each analysis methodology, are set out in Table 5.

TABLE 4

| Gene | | | Entrez Gene ID and |
|---|---|---|---|
| symbol | Name | Synonyms | protein sequence link |
| ZMAT2 | Zinc finger, matrin type 2 | FLJ31121 | 153527; |
| | | | http://ca.expasy.org/uniprot/Q96NC0 |
| BANK1 | B-cell scaffold protein with | BANK; FLJ20706; FLJ34204 | 55024; |
| | ankyrin repeats 1 | | http://ca.expasy.org/uniprot/Q8NDB2 |
| BPY2IP1 | MAP1S protein | MAP8; C19orf5; VCY2IP1; | 55201; |
| MAP1S | | FLJ10669; VCY2IP-1; | http://ca.expasy.org/uniprot/Q66K74 |
| | | MGC133087 | |
| CEBPG | CCAAT/enhancer binding | GPE1BP; IG/EBP-1; CEBPG | 1054; |
| | protein (C/EBP), gamma | | http://ca.expasy.org/uniprot/P53567 |
| E1B-AP5 | E1B-55 kDa-associated | E1BAP5; E1B-AP5; | 11100; |
| | protein 5 | HNRPUL1; FLJ12944 | http://ca.expasy.org/uniprot/O76022 |
| FUS | Fusion (involved in t(12;16) | TLS; CHOP; FUS1; FUS- | 2521; |
| | in malignant liposarcoma) | CHOP; TLS/CHOP; hnRNP- | http://ca.expasy.org/uniprot/P35637 |
| | | P2 | |
| HMG20B | High-mobility group 20B | SOXL; HMGX2; BRAF25; | 10362; |
| | | BRAF35; PP7706; pp8857; | http://ca.expasy.org/uniprot/Q9P0W2 |
| | | FLJ26127; SMARCE1r | |
| IFI16 | Interferon, gamma-inducible | PYHIN2; IFNGIP1; MGC9466 | 3428; |
| | protein 16 | | http://ca.expasy.org/uniprot/Q16666 |
| KRT8 | Keratin 8 | K8; KO; CK8; CYK8; K2C8; | 3856; |
| | | CARD2 | http://ca.expasy.org/uniprot/P05787 |
| LIN28 | Lin-28 homolog (C. elegans) | CSDD1; LIN-28; LIN28A; | 79727; |
| | | ZCCHC1; FLJ12457 | http://ca.expasy.org/uniprot/Q9H9Z2 |
| PABPC1 | Poly(A) binding protein, | PAB1; PABP; PABP1; | 26986; |
| | cytoplasmic 1 | PABPC2; PABPL1 | http://ca.expasy.org/uniprot/P11940 |
| PHLDA1 | Pleckstrin homology-like | PHRIP; TDAG51; DT1P1B11; | 22822; |
| | domain, family A, member 1 | MGC131738 | http://ca.expasy.org/uniprot/Q8WV24 |
| PIAS2 | Msx-interacting-zinc finger, | miz; MIZ1; SIZ2; PIASX; | 9063; |
| | transcript variant alpha | ZMIZ4; MGC102682; PIASX- | http://ca.expasy.org/uniprot/Q13105 |
| | | BETA; PIASX-ALPHA | |
| PSME3 | Proteasome (prosome, | Ki; PA28G; REG-GAMMA; | 10197; |
| | macropain) activator | PA28-gamma | http://ca.expasy.org/uniprot/Q12920 |
| | subunit 3 (PA28 gamma; Ki) | | |
| RALBP1 | RalA binding protein 1 | RIP; RIP1; RLIP76 | 10928; |
| | | | http://ca.expasy.org/uniprot/Q15311 |
| RARA | Retinoic acid receptor, | RAR; NR1B1 | 5914; |
| | alpha | | http://ca.expasy.org/uniprot/P10276 |
| RDBP | RD RNA binding protein | RD; RDP; D6S45; NELF-E | 7936; |
| | | | http://ca.expasy.org/uniprot/P18615 |
| SRPK1 | SFRS protein kinase 1 | SFRSK1 | 6732; |
| | | | http://ca.expasy.org/uniprot/Q96SB4 |
| SSA2 | Sjogren syndrome antigen | RO60; SSA2; TROVE2 | 6738; |
| | A2 (60 kDa, | | http://ca.expasy.org/uniprot/P10155 |
| | ribonucleoprotein | | |
| | autoantigen SS-A/Ro) | | |
| STAU | Staufen, RNA binding | STAU; FLJ25010 | 6780; |
| | protein (Drosophila), | | http://ca.expasy.org/uniprot/O95793 |
| | transcript variant T3 | | |
| TXNL2 | Thioredoxin-like, clone | GRX3; GRX4; GLRX4; | 10539; |
| | MGC: 12349 | PICOT; TXNL2; TXNL3; | http://ca.expasy.org/uniprot/O76003 |
| | | FLJ11864; bA500G10.4; | |
| | | GLRX3 | |
| ZNF38 | Zinc finger protein 38 | ZNF38; Zipro1; NY-REN-21; | 7589; |
| | | DKFZp434L134; | http://ca.expasy.org/uniprot/Q9Y5A6 |
| | | DKFZp686H10254 | |

TABLE 5

| Gene | | | Entrez Gene ID and |
|---|---|---|---|
| symbol | Name | Synonyms | protein sequence link |
| ASPSCR1 | alveolar soft part sarcoma | TUG; ASPL; ASPS; RCC17; | 79058; |
| | chromosome region, | UBXD9; UBXN9; ASPCR1; | http://ca.expasy.org/uniprot/Q7Z6N7 |
| | candidate 1 | FLJ45380; ASPSCR1 | |
| DDX55 | DEAD (Asp-Glu-Ala-Asp) | FLJ16577; KIAA1595; | 57696; |
| | box polypeptide 55 | MGC33209; DDX55 | http://ca.expasy.org/uniprot/Q8NHQ9 |
| DOM3Z | dom-3 homolog Z (C. elegans) | NG6; DOM3L; DOM3Z | 1797; |
| | | | http://ca.expasy.org/uniprot/O77932 |
| HAGH | Hydroxyacylglutathione | GLO2; GLX2; GLXII; HAGH1 | 3029; |
| | hydrolase | | http://ca.expasy.org/uniprot/Q16775 |
| HOXB6 | Homeo box B6, transcript | HOX2; HU-2; HOX2B; Hox- | 3216; |
| | variant 2 | 2.2 | http://ca.expasy.org/uniprot/P17509 |
| LNX | ligand of numb-protein X | LNX; MPDZ; PDZRN2; LNX1 | 84708; |
| | | | http://ca.expasy.org/uniprot/Q8TBB1 |
| NDUFV3 | NADH dehydrogenase | CI-9KD | 4731; |
| | (ubiquinone) flavoprotein | | http://ca.expasy.org/uniprot/P56181 |
| | 3, 10 kDa | | |
| PRKCBP1 | protein kinase C binding | RACK7; PRKCBP1; | 23613; |
| | protein 1 | PRO2893; MGC31836; | http://ca.expasy.org/uniprot/Q9H2G5 |
| | | ZMYND8 | |
| PRKRA | protein kinase, interferon- | RAX; PACT; DYT16; HSD14; | 8575; |
| | inducible double stranded | PRKRA | http://ca.expasy.org/uniprot/O75569 |
| | RNA dependent activator | | |
| RAB11FIP3 | RAB11 family interacting | KIAA0665; Rab11-FIP3 | 9727; |
| | protein 3 (class II) | | http://ca.expasy.org/uniprot/O75154 |
| RAN | RAN, member RAS | TC4; Gsp1; ARA24 | 5901; |
| | oncogene family | | http://ca.expasy.org/uniprot/P17080 |
| RBMS1 | RNA binding motif, single | YC1; MSSP; SCR2; MSSP-1; | 5937; |
| | stranded interacting | MSSP-2; MSSP-3; | http://ca.expasy.org/uniprot/P29558 |
| | protein 1, transcript | MGC3331; MGC15146 | |
| | variant | | |
| RNF12 | Ring finger protein 12, | RLIM; MGC15161; NY-REN- | 51132; |
| | transcript variant 1 | 43 | http://ca.expasy.org/uniprot/Q9NVW2 |
| RPL30 | ribosomal protein L30 | RPL30 | 6156; |
| | | | http://ca.expasy.org/uniprot/P04645 |
| RPL31 | ribosomal protein L31 | MGC88191; RPL31 | 6160; |
| | | | http://ca.expasy.org/uniprot/P62899 |
| RUFY1 | RUN and FYVE domain | RABIP4; ZFYVE12; | 80230; |
| | containing 1 | FLJ22251 | http://ca.expasy.org/uniprot/Q96T51 |
| SMN1 | Survival of motor neuron | SMA; SMN; SMA1; SMA2; | 6606; |
| | 1, telomeric | SMA3; SMA4; SMA@; | http://ca.expasy.org/uniprot/Q16637 |
| | | SMNT; BCD541; T-BCD541 | |
| SNK | serum-inducible kinase | SNK; PLK2 | 10769; |
| | | | http://ca.expasy.org/uniprot/Q9NYY3 |
| SSNA1 | Sjogren's syndrome | N14; NA14; NA-14 | 8636; |
| | nuclear autoantigen 1 | | http://ca.expasy.org/uniprot/O43805 |
| STK11 | Serine/threonine kinase | PJS; LKB1 | 6794; |
| | 11 (Peutz-Jeghers | | http://ca.expasy.org/uniprot/Q15831 |
| | syndrome) | | |
| TOM1 | Target of myb1 (chicken) | FLJ33404 | 10043; |
| | | | http://ca.expasy.org/uniprot/O60784 |
| TXNRD1 | Thioredoxin reductase 1, | TR; TR1; TXNR; TRXR1; | 7296; |
| | transcript variant 5 | GRIM-12; MGC9145 | http://ca.expasy.org/uniprot/Q16881 |
| VCL | vinculin | MVCL; CMD1W; VCL | 7414; |
| | | | http://ca.expasy.org/uniprot/P18206 |

In other embodiments, a method is provided which is useful in diagnosing a patient as having or being predisposed to developing lupus, comprising testing a sample from the patient against a panel of auto-antigens to detect the presence, absence or a modulated level of auto-antibodies in the sample, or in the panel of auto-antigens comprises at least 10 auto-antigens selected from Table 3. In other embodiments, the at least 10 auto-antigens may be selected from Table 1, or from Table 2. Preferably a panel comprises at least 20 auto-antigens. Optionally a panel may comprise 11, 12, 13, 14, 15, 16, 17, 18, 19, 20, 21, 22, 23, 24, 25, 26, 27, 28, 29 or 30 or more auto-antigens.

The panel of auto-antigens useful in the diagnosis of lupus may comprise the auto-antigens set out in Table 1, or in Table 2, or in Table 3. In some embodiments the panel may have up to 5 auto-antigens deleted or replaced, or optionally up to 6, 7, 8, 9 or 10 or more auto-antigens deleted or replaced provided the required specificity and selectivity of the method is retained.

It is expected that a clinically valuable panel of biomarkers should be large enough to allow a disease to be detected accurately, minimizing false positives and false negatives, whilst also being small enough to be practical.

In embodiments the methods of the present invention use a panel of auto-antigens comprising the auto-antigens listed in Table 4. Such a method may use a panel comprising 1 or more additional auto-antigens selected from the group consisting of the auto-antigens of Table 5.

In the methods of the present invention detecting the presence or an increased level of at least 2 auto-antibodies provides information useful for diagnosis. Optionally, detecting the presence of an increased level of at least 3, preferably at least 4 and more preferably at least 5 auto-antibodies provides information useful for diagnosis.

By analysing the relationship between the panel of biomarkers and the status of the samples used to derive the panel, it is possible to derive a mathematical relationship between the presence, absence or a modulated level of auto-bodies directed against proteins in the panel and the presence, absence, progression or regression of lupus. The application of this mathematical relationship to naive samples to be tested can be used to derive information useful for diagnosis, progression or regression of lupus.

Where it is not possible to derive such a mathematical relationship, it is possible to derive a statistical relationship between the presence, absence or modulated level of auto-antibodies directed against proteins in the panel and the presence, absence, progression or regression of lupus. The application of this statistical relationship to naive sample to be tested can be used to derive information useful for diagnosis, progression or regression of lupus.

According to a different aspect of the present invention there is provided a diagnostic kit for use in diagnosing a patient as having or being predisposed to developing lupus, or having presymptomatic lupus, said kit comprising one or more auto-antigens selected from the group consisting of ZMAT2 (Zinc finger, matrin type 2); BANK1 (B-cell scaffold protein with ankyrin repeats 1); BPY2IP1 (MAP1S protein); CEBPG (CCAAT/enhancer binding protein (C/EBP), gamma); E1B-AP5 (E1B-55 kDa-associated protein 5); FUS (Fusion (involved in t(12;16) in malignant liposarcoma)); HAGH (Hydroxyacylglutathione hydrolase); HMG20B (High-mobility group 20B); HOXB6 (Homeo box B6, transcript variant 2); KRT8 (Keratin 8); L1N28 (Lin-28 homolog (C. elegans)); NDUFV3 (NADH dehydrogenase (ubiquinone) flavoprotein 3, 10 kDa); PABPC1 (Poly(A) binding protein, cytoplasmic 1); PHLDA1 (Pleckstrin homology-like domain, family A, member 1); PIAS2 (Msx-interacting-zinc finger, transcript variant alpha); RAB11FIP3 (RAB11 family interacting protein 3 (class II)); RAN (RAN, member RAS oncogene family); RARA (Retinoic acid receptor alpha); RBMS1 (RNA binding motif, single stranded interacting protein 1, transcript variant); RDBP (RD RNA binding protein); RNF12 (Ring finger protein 12, transcript variant 1); RUFY1 (RUN and FYVE domain containing 1); SMN1 (Survival of motor neuron 1, telomeric); SRPK1 (SFRS protein kinase 1); SSNA1 (Sjogren's syndrome nuclear autoantigen 1); STAU (Staufen, RNA binding protein (Drosophila), transcript variant T3); STK11_(Serine/threonine kinase 11 (Peutz-Jeghers syndrome)); TOM1 (Target of myb1 (chicken)); TXNL2 (Thioredoxin-like, clone MGC:12349); TXNRD1 (Thioredoxin reductase 1, transcript variant 5); or ZNF38 (Zinc finger protein 38), which one or more auto-antigens are immobilised on a substrate.

In one embodiment, the one or more auto-antigens includes ZMAT2.

In addition to one or more of the auto-antigens above, the kit may comprise one or more of the auto-antigens BANK1 (B-cell scaffold protein with ankyrin repeats 1); IFI16 (Interferon, gamma-inducible protein 16); PSME3 (Proteasome (prosome, macropain) activator subunit 3 (PA28 gamma; Ki), transc); RALBP1 (RalA-binding protein 1); and SSA2 (Sjogren syndrome antigen A2 (60 kDa, ribonucleoprotein autoantigen SS-A/Ro)).

Alternatively, said kit may comprise a panel comprising 5 or more, preferably 6, 7, 8, 9, 10, 11, 12, 13, 14, 15, 16, 17, 18, 19, 20, 21, 22, 23, 24, 25, 26, 27, 28, 29, 30, 31, 32, 33, 34 or more, even more preferably to a panel comprising all 35, auto-antigens selected from the auto-antigens listed in Table 1, wherein the auto-antigens are immobilised on a substrate.

According to another aspect of the present invention a diagnostic kit is provided for use in diagnosing a patient as having or being predisposed to developing lupus, or having pre-symptomatic lupus, said kit comprising one or more auto-antigens selected from the group of auto-antigens of Table 2.

Alternatively, said kit may comprise a panel comprising 5 or more, preferably 6, 7, 8, 9, 10, 11, 12, 13, 14, 15, 16, 17, 18, 19, 20, 21, 22, 23, 24, 25, 26, 27, 28, 29, 30, 31 or more, even more preferably to a panel comprising all 32 auto-antigens selected from the auto-antigens listed in Table 2, wherein the auto-antigens are immobilised on a substrate.

According to another aspect of the present invention there is provided a diagnostic kit for use in diagnosing a patient as having or being predisposed to developing lupus, or having pre-symptomatic lupus, said kit comprising 1 or more auto-antigens selected from the group of auto-antigens of Table 3.

Alternatively said kit may comprise a panel comprising 5 or more, preferably, 6, 7, 8, 9, 10, 11, 12, 13, 14, 15,16, 17, 18, 19, 20, 21, 22, 23, 24, 25, 26, 27, 28, 29, 30, 31, 32, 33, 34, 35, 36, 37, 38, 39, 40, 41, 42, 43, 44 or more, even more preferably to a panel comprising all 45 auto-antigens selected from the auto-antigens listed in Table 3, wherein the auto-antigens are immobilised on a substrate.

In some embodiments, said substrate may comprise a strip, a slide, beads or a well of a microtitre plate or a “chip” for use with Surface Plasmon Resonance (Biacore) or planar waveguide technology. Suitably, the substrate may comprise a disposable test strip of the kind widely used in diagnostic kits for use at the point of care.

The panel of auto-antigens may be carried on said substrate in the form of an addressable array. Said array may be a microarray. The auto-antigens of the panel may constitute at least 5%, 10%, 15%, 20%, 25%, 40% 50%, typically at least 70% or 80%, of the total number of proteins present on the array. For example, the auto-antigens of the panel may constitute more than 85%, 90% or 95% of the total number of proteins on the array.

Alternatively, said diagnostic kit may comprise said panel of auto-antigens carried on a substrate in the form of an array, which array includes one or more other proteins or polypeptides, and means for automatically analysing the results of tests performed using said array, said analysing means including computer-readable instructions in which results from said one or more other proteins or polypeptides are blinded during the analysis.

In addition to the auto-antigens (and optional one or more other proteins or polypeptides) forming said panel, the array may further comprise one or more control proteins or polypeptides. Suitably, the array may comprise one or more replicates of the auto-antigens or controls, preferably replicates of each auto-antigen and each control.

Advantageously, the array comprises an array of correctly folded auto-antigens, which may be prepared in accordance with the methods disclosed by WO-A-01/57198 or WO-A-02/27327, for example, the contents of which are incorporated herein by reference. Preferably, the array comprises an array of correctly folded tagged auto-antigens that are attached to said substrate via the tag, which array may be prepared, for example, in accordance with the methods disclosed by WO 03/064656 or WO-A-2004/046730, the contents of which are also incorporated herein by reference.

Said kit may further comprise an anti-human immunoglobulin antibody or other protein that specifically binds immunoglobulin, and means for detecting the anti-human immunoglobulin antibody or other immunoglobulin-binding protein.

Said detection means may comprise a colorimetric or fluorescent assay or utilise label-free methods such as surface plasmon resonance, mass spectrometry or planar waveguide technology.

The anti-human immunoglobulin antibody may be an anti-human IgM antibody, an anti-human IgG antibody, an anti-human IgA antibody, an anti-human IgE antibody or a combination of any two or more of such anti-human antibodies; preferably, the anti-human immunoglobulin antibody is an anti-human IgG antibody. Alternatively, or additionally, a protein that specifically binds an antibody is included in the kit. Preferably, this protein is protein A or protein G, or a recombinant form thereof.

According to yet another aspect of the present invention there is provided the use of one or more auto-antigens selected from the aforementioned groups as shown in Tables 1, 2, 3 or 4, or antigenic fragments or antigenic epitopes thereof, or a nucleic acid encoding the auto-antigen or fragment or epitope thereof, as an active agent in the manufacture of a vaccine for the treatment or prevention of lupus. Immunization with such an active agent may be protective against aberrant expression of the said antigen in lupus. In one embodiment the vaccine comprises ZMAT2, optionally in combination with one or more further antigens specified in Tables 1, 2, 3 or 4.

Said active agent may be formulated in a pharmaceutical composition in accordance with method is well known in the art. Said composition may further comprise a pharmaceutically acceptable vehicle. Said vehicle may comprise one or more excipients, carriers, buffers, stabilisers or other materials well known to those skilled in the art. Such materials should be non-toxic and should not interfere with the efficacy of the active agent. The precise nature of the carrier or other material may depend on the route of administration, e.g., oral, intravenous, cutaneous or subcutaneous, nasal, intramuscular, intraperitoneal or patch routes.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Whether it is a polypeptide, peptide, or nucleic acid molecule, other pharmaceutically useful compound according to the present invention that is to be given to an individual, administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in REMINGTON'S PHARMACEUTICAL SCIENCES, 16th edition, Osol, A. (ed), 1980.

Alternatively, targeting therapies may be used to deliver the active agent more specifically to certain types of cell, by the use of targeting systems such as antibody or cell specific ligands. Targeting may be desirable for a variety of reasons; for example if the agent is unacceptably toxic, or if it would otherwise require too high a dosage, or if it would not otherwise be able to enter the target cells.

Instead of administering these agents directly, they could be produced in the target cells by expression from an encoding gene introduced into the cells, e.g. in a viral vector (a variant of the VDEPT technique—see below). The vector could be targeted to the specific cells to be treated, or it could contain regulatory elements, which are switched on more or less selectively by the target cells.

Alternatively, the agent could be administered in a precursor form, for conversion to the active form by an activating agent produced in, or targeted to, the cells to be treated. This type of approach is sometimes known as ADEPT or VDEPT; the former involving targeting the activating agent to the cells by conjugation to a cell-specific antibody, while the latter involves producing the activating agent, e.g. a vaccine or fusion protein, in a vector by expression from encoding DNA in a viral vector (see for example, EP-A-415731 and WO 90/07936).

The vaccines of the present invention also include one or more adjuvant compounds, i.e. a compound or compounds that increases an immunogenic response or the immunogenicity of an antigen or vaccine. Adjuvant compounds useful in the present invention include Complete Freund's Adjuvant (CFA); Incomplete Freund's Adjuvant (IFA); Montanide ISA (incomplete seppic adjuvant); Ribi Adjuvant System (RAS); TiterMax; Syntex Adjuvant Formulation (SAF); Aluminum Salt Adjuvants; Nitrocellulose-adsorbed antigen; Encapsulated or entrapped antigens; Immune-stimulating complexes (ISCOMs); and Gerbu^{R} adjuvant.

Following is a description by way of example only with reference to the accompanying drawings of embodiments of the present invention.

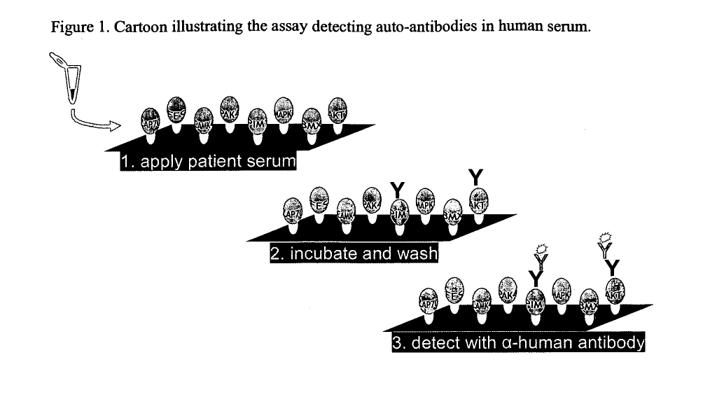

In the drawings, FIG. 1 is a cartoon illustrating a method for the detection of auto-antibodies in serum.

### EXAMPLES

### Example 1

### A Microarray of Antigens for Discovering Auto-Antibody Biomarkers in Patient Serum that are Diagnostic for Lupus

Full-length open reading frames for 330 target genes covering a wide range of protein classes were cloned in-frame with a sequence encoding a C-terminal E. coli BCCP-myc tag (WO 03/064656; Boutell) in a baculovirus transfer vector and sequence-verified. Recombinant baculoviruses were generated, amplified and expressed in Sf9 cells grown in suspension using standard methods adapted for 24-well deep well plates (Whatman, Maidstone, UK; Chambers, Zhao). Recombinant protein expression was analyzed for protein integrity and biotinylation by Western blotting. Cells harboring recombinant protein were lysed and lysates were spotted in quadruplicate using a QArray2 Microarrayer (Genetix, New Milton, UK) equipped with 300 μm solid pins on to streptavidin-coated glass slides (Schott Nexterion, Jena, Germany). Slides were stored at −20° C. A range of protein activities have been demonstrated to be stable under these conditions for over 12 months. Proteins spotted on the array project into an aqueous environment and orient away from the surface of the glass chips, exposing them for binding by auto-antibodies.

In addition to the 330 proteins, four control proteins for the BCCP-myc tag (BCCP, BCCP-myc, β-galactosidase-BCCP-myc and β-galactosidase-BCCP) were arrayed, along with Cy3/Cy5-labeled biotin-BSA, dilution series of biotinylated-IgG and biotinylated IgM, a biotinylated-myc peptide dilution series and buffer only spots.

### Example 2

### Detection of Auto-Antibodies in Serum Samples from Patients Suffering from Lupus

Serum samples of 169 individuals diagnosed with lupus (median age 47 years, 95% females) were obtained from 2 commercial sources. As controls, samples of 170 individuals with no known history of lupus (median age 50 years, 95% females) were obtained. These samples were assayed for the presence of auto-antibodies using the protein arrays described in Example 1.

Serum samples were clarified (centrifuged at 10-13K rcf for 3 minutes at 4° C. to remove particulates, including lipids) and diluted 200-fold in 0.1% v/v Triton/0.1% w/v BSA in 1× PBS (Triton-BSA-PBS buffer) and then applied to the arrays. Each diluted serum sample (2.0 mL) was applied to a separate array and incubated in a dish for 2 hours at room temperature (RT, 20° C.) with gentle orbital shaking 50 rpm). Arrays were then carefully removed from the dish and any excess probing solution was removed by blotting the sides of the array onto lint-free tissue. Probed arrays were washed twice in fresh Triton-BSA-PBS buffer in a new plastic container with screw-on lid at room temperature for 5 minutes with rapid orbital shaking. The washed slides were then blotted onto lint-free tissue to remove excess wash buffer and were incubated in a secondary antibody solution (prepared just prior to use) at room temperature for 2 hours, with gentle orbital shaking and protected from light. The secondary antibody solution was a Cy3 labeled rabbit anti-human IgG antibody diluted in Triton-BSA-PBS buffer to give optimal detection of bound serum IgG. The slides were washed three times in Triton-BSA-PBS buffer for 5 minutes at RT with gentle orbital shaking, rinsed briefly (5-10 seconds) in distilled water, and centrifuged for 2 minutes at 240 rcf in a container suitable for centrifugation. To help wick away excess liquid on the arrays, a lint-free tissue was placed at the bottom of the arrays during centrifugation.

The probed and dried arrays were then scanned using a microarray scanner capable of using an excitation wavelength suitable for the detection of the secondary staining solution, such as the Molecular Devices Genepix 4000B microarray scanner, to identify auto-antibodies bound by the array and to determine intensity of auto-antibody binding. The microarray scans produced TIFF images for each array that were used to determine the intensity of fluorescence bound to each protein spot.

Raw median signal intensity (also referred to as the relative fluorescent unit, RFU) of each protein feature (also referred to as a spot or antigen) on the array was determined from the TIFF images using GenePix Pro microarray data analysis software or similar software and subtracted from the local median background intensity. It is also possible to use other measures of spot intensity such as the mean fluorescence, total fluorescence, as is known by anyone skilled in the art.

The resulting net fluorescent intensities of all protein features on each array were then normalized using the quantile normalization (Bolstad) method. Data for sera from two lupus patients and one control serum were excluded because many of their antigen binding signals were masked by high background binding. Other methods for data normalization suitable for the current data include, amongst others, multiplication of net fluorescent intensities by a normalization factor consisting of the product of the 1^{st} quartile of all intensities of a sample and the mean of the 1^{st} quartiles of all samples and the vsn method (Huber). Such normalization methods are known to anyone skilled in the art of microarray analysis. The normalized fluorescent intensities were then averaged for each protein feature.

The normalized mean data were used to select biomarkers for lupus. The data were randomly split into training and test sets 10 times (10 fold cross-validation)

Proteins were selected and ranked with the significance analysis of microarrays (SAM) algorithm (Tusher) using the data in the training subset. Similar methods that are applicable to this task include, amongst others, Prediction Analysis of Microarrays (PAM, Tibshirani), MP test (Fox), fold change (Witten) and principal component analysis (Bessant). Such data analysis methods are known to anyone skilled in the art of microarray analysis. The data were analysed as two class—unpaired (diseased vs control) using a non-parametric test statistic. The protein features selected in each cross-validation fold with the highest scores and a q-value of ≦30% were selected and those with q-values≦30% in most of the 10 cross-validation iterations were selected and are shown in Table 2 alongside the frequency of selection in cross-validation.

Table 1a lists the protein features for which the q-value was ≦30% by SAM analysis alongside the selection frequency in cross-validation

| Gene symbol | cross-validation selection frequency |
|---|---|
| ZNF38 | 10 |
| BPY2IP1 | 10 |
| CEBPG | 10 |
| E1B-AP5 | 10 |
| FUS | 10 |
| HMG20B | 10 |
| LIN28 | 10 |
| NDUFV3 | 10 |
| PABPC1 | 10 |
| PHLDA1 | 10 |
| PIAS2 | 10 |
| PSME3 | 10 |
| RAB11FIP3 | 10 |
| RALBP1 | 10 |
| RAN | 10 |
| RARA | 10 |
| RBMS1 | 10 |
| RNF12 | 10 |
| SSA2 | 10 |
| STAU | 10 |
| STK11 | 10 |
| TOM1 | 10 |
| ZMAT2 | 10 |
| SRPK1 | 9 |
| TXNRD1 | 9 |
| BANK1 | 8 |
| IFI16 | 8 |
| KRT8 | 8 |
| RUFY1 | 8 |
| SMN1 | 8 |
| SSNA1 | 8 |
| HAGH | 7 |
| HOXB6 | 7 |
| RDBP | 7 |
| TXNL2 | 7 |

The proteins listed in Table la are more frequently bound by sera from diseased than control subjects. The proteins listed in Table la are considered as biomarkers useful in diagnosis of lupus.

The normalized data of the training set for the proteins selected in each cross-validation fold were used to construct a classification algorithm. An algorithm was established based on using logistic regression (Liao) algorithms. Similar methods that can be used to construct classification algorithms for the sample data of this study include, amongst others, PAM (Tibshirani), artificial neural networks (Bessant), support vector machine (SVM) (Brown) and regularized discriminant analysis (Guo). Such data analysis methods are known to anyone skilled in the art of microarray analysis. The constructed algorithm was then used to classify the samples of the test set into diseased and control samples in order to check its validity as classification method for lupus. In order to get an estimate of the classification errors the mean specificity and sensitivity were examined using 10 fold cross validation. The cross validation mean sensitivity was 67%, the mean specificity was 85%. The cross validation approach ensured robust measurements of classification errors.

### Example 3

### Detection of Auto-Antibodies in Serum Samples from Patients Suffering from Lupus Using a Modified Analysis Approach

The raw data (169 lupus and 170 controls samples) as described in Example 2 was subjected to modified analysis in order to mine the data still further and identify additional markers indicative of lupus.

Raw median signal intensity (relative fluorescent unit, RFU) of each protein feature on the array was determined from the TIFF images using ArrayPro microarray data analysis software and subtracted from the local median background intensity.

The resulting net fluorescent intensities of all protein features from all three array types were then normalized together using the vsn normalization method (Huber 2002), or multiplication of net fluorescent intensities by a normalization factor consisting of the product of the 1st quartile of all intensities of a sample and the mean of the 1st quartiles of all samples, or the quantile or robust quantile method (Bolstad 2003). The normalized fluorescent intensities were then averaged for each protein feature.

The normalized mean data were used to select biomarkers for lupus. In order to estimate the specificity and sensitivity of a classification method based on biomarkers selected, data were initially split randomly into training and test sets 10 times (10 fold cross-validation).

Proteins were selected and ranked with the MP test (Fox 2006), or Prediction Analysis of Microarrays (PAM, Tibshirani 2002), significance analysis of microarrays (SAM) algorithm (Tusher 2001, Hueber 2005), fold change (Witten 2007), or a cut-off method based on a confidence interval derived from the data of the controls, using the data in the training subset. The data were analysed as two class (diseased vs control).

The normalized data of the training set for the proteins selected in each cross-validation fold were used to construct a classification algorithm. Features derived from the same clones that were selected on more than one array type were left in for the classification algorithm construction. An algorithm was established based on penalised logistic regression (Antoniadis 2003, Liao 2007), or PAM (Tibshirani 2002), or support vector machine (SVM, Brown 2000, Statnikov 2008) or a cut-off method based on a confidence interval derived from the data of the controls. The constructed algorithms were then used to classify the samples of the test set into diseased and control samples in order to check its validity as classification method for lupus. In order to get an estimate of the classification errors the mean specificity and sensitivity were examined using 10 fold cross validation.

The proteins selected by SAM analysis in at least 9 of the 10 cross validation folds are shown in Table 2a. The analysis parameters used were: q-value cut-off ≦20% and proteins with elevated responses included.

Table 2a lists the protein features for which the q-value was ≦20% by SAM analysis and 10 fold cross validation selection frequency ≧9.

| Gene symbol | Selection frequency |
|---|---|
| ASPSCR1 | 10 |
| BANK1 | 10 |
| CEBPG | 10 |
| DDX55 | 10 |
| DOM3Z | 9 |
| E1B-AP5 | 10 |
| FUS | 10 |
| HMG20B | 10 |
| IFI16 | 10 |
| KRT8 | 10 |
| LIN28 | 10 |
| LNX | 10 |
| MAP1S | 10 |
| PABPC1 | 10 |
| PHLDA1 | 10 |
| PIAS2 | 10 |
| PRKCBP1 | 10 |
| PRKRA | 10 |
| PSME3 | 10 |
| RALBP1 | 10 |
| RARA | 10 |
| RDBP | 10 |
| RPL31 | 9 |
| RPL30 | 10 |
| SNK | 10 |
| SRPK1 | 10 |
| SSA2 | 10 |
| STAU1 | 10 |
| TXNL2 | 10 |
| VCL | 10 |
| ZMAT2 | 9 |
| ZNF38 | 9 |

The proteins listed in Table 2a are more frequently bound by sera from diseased than control subjects. The proteins listed in Table 2a are considered as biomarkers useful in diagnosis of lupus.

Classification by SVM gave a cross validation mean sensitivity of 74% and a mean specificity of 86%.

### REFERENCES

Antoniadis, A. Penalized Logistic Regression and Classification of Microarray Data. Bioconductor Workshop Milan 2003 (http://www.bioconductor.org/workshops/2003/Milan/Lectures/anestisMilan3.pdf).

Bessant, C. Microarray Analysis Software and its Applications, in The Handbook of

Biosensors and Biochips (Eds. R. Marks, D. Cullen, I. Karube, H. Weetall, C. Lowe), Wiley, 2007.

Bolstad B M, Irizarry R A, Astrand M, Speed T P. A comparison of normalization methods for high density oligonucleotide array data based on variance and bias. Bioinformatics. 2003 Jan. 22; 19(2):185-93.

Boutell, J. M., Hart, D. J., Godber, B. L., Kozlowski, R. Z. and Blackburn, J. M. Functional protein microarrays for parallel characterisation of p53 mutants. Proteomics 2004 4(7), 1950-1958

Brown M P, Grundy W N, Lin D, Cristianini N, Sugnet C W, Furey T S, Ares M Jr, Haussler D. Knowledge-based analysis of microarray gene expression data by using support vector machines. Proc Natl Acad Sci U S A. 2000 Jan. 4; 97(1):262-7.

Chambers S. P., Austen D. A., Fulghum J. R., Kim W. M. High-throughput screening for soluble recombinant expressed kinases in Escherichia coli and insect cells. Protein Expr Purif. 2004 36(1):40-7

Fox R J, Dimmic M W. A two-sample Bayesian t-test for microarray data.BMC Bioinformatics. 2006 Mar. 10; 7:126.

Guo, Y. et al. (2004) Regularized Discriminant Analysis and Its Application in Microarrays, Technical Report, Department of Statistics, Stanford University.

Huber W, von Heydebreck A, Scultmann H, Poustka A, and Vingron M. Variance stablization applied to microarray data calibration and to quantification of differential expression. Bioinformatics, 18:S96 { S104, 2002.

Liao J G, Chin K V. Logistic regression for disease classification using microarray data: model selection in a large p and small n case. Bioinformatics. 2007 Aug. 1; 23(15):1945-51.

Statnikov A, Wang L, Aliferis C F. A comprehensive comparison of random forests and support vector machines for microarray-based cancer classification. BMC Bioinformatics. 2008 Jul. 22; 9:319.

Tibshirani R, Hastie T, Narasimhan B, Chu G. Diagnosis of multiple cancer types by shrunken centroids of gene expression. Proc Natl Acad Sci U S A. 2002 May 14; 99(10):6567-72.

Tusher, Tibshirani and Chu (2001). Significance analysis of microarrays applied to the ionizing radiation response. PNAS 2001 98: 5116-5121.

Witten, D. and Tibshirani, R. A comparison of fold change and the t-statistic for microarray data analysis. Technical Report, Stanford University, 2007.

Zhao Y, Chapman D A, Jones I M. Improving baculovirus recombination. Nucleic Acids Res. 200331(2):E6-6

### Gene Symbol, Entrez Gene ID, DNA Accession Number and Sequence of Antigens.

Gene symbol: ASPSCR1
Entrez Gene ID: 79058
dna/ac: BC018722.1
sequence:
(SEQ ID NO: 1)
atggcggccccggcaggcggcggaggctccgcggtgtcggtgctggcc
ccgaacggccggcgccacacggtgaaggtgacgccgagcaccgtgctg
cttcaggttctggaggacacgtgccggcggcaggacttcaacccctgt
gaatatgatctgaagtttcagaggagcgtgctcgacctttctctccag
tggagatttgccaacctgcccaacaatgccaagctggagatggtgccc
gcttcccggagccgtgaggggcctgagaacatggttcgcatcgctttg
cagctggacgatggctcgaggttgcaggactattctgttcaggccaga
ccctctgggagatctcagccattttccacagatcagggagtgcctgca
gcaccccggcggggccaccccagtctgcgtgtacacgagggatgaggt
gacgggtgaagctgccctgcggggcacgacgctgcagtcgctgggcct
gaccgggggcagcgccaccatcaggtttgtcatgaagtgctacgaccc
cgtgggcaagaccccaggaagcctgggctcgtcagcgtcggctggcca
ggcagccgccagcgctccacttccatggaatctggggagctcagccgc
ggcgacttgagccgtccggaggacgcggacacctcagggccctgctgc
gagcacactcaggagaagcagagcacaagggcacccgcagctgcccca
ttgttcattctcgggtgggggacagagacaggggggccctcctgggcc
cacgaggcctctgacatcatcttcagctaagttgccgaagtccctctc
cagccctggaggcccctccaagccaaagaagtccaagtcgggccagga
tccccagcaggagcaggagcaggagcgggagcgggatccccagcagga
gcaggagcgggagcggcccgtggaccgggagcccgtggaccgggagcc
ggtggtgtgccaccccgacctggaggagcggctgcaggcctggccagc
ggagctgcctgatgagttattgagctgacggtggacgacgtgagaaga
cgcttggcccagctcaagagtgagcggaagcgcctggaagaagccccc
ttggtgaccaaggccttcagggaggcgcagataaaggagaagctggag
cgctacccaaaggtggctctgagggtcctgttccccgaccgctacgtc
ctacagggcttatccgccccagcgagacagtgggggacttgcgagact
tcgtgaggagccacctggggaaccccgagctgtcattttacctgttca
tcacccctccaaaaacagtcctggacgaccacacgcagaccctctttc
aggcgaacctcttcccggccgctctggtgcacttgggagccgaggagc
cggcaggtgtctacctggagcctggcctgctggagcatgccatctccc
catctgcggccgacgtgctggtggccaggtacatgtccagggccgccg
ggtccccttccccattgccagcccctgaccctgcacctaagtctgagc
cagctgctgaggagggggcgctggtcccccctgagcccatcccaggga
cggcccagcccgtgaagaggagcctgggcaaggtgcccaagtggctga
agctgccggccagcaagaggtga
Gene symbol: BANK1
Entrez Gene ID: 55024
dna/ac: BC032241.1
sequence:
(SEQ ID NO: 2)
atgctgccagcagcgccaggcaaggggcttgggagcccggaccccgcc
ccctgcggcccagcgcccccaggaaatacaaaagatataataatgata
tatgaagaagatgctgaggaatgggctctgtacttgacagaagtattt
ttacatgttgtgaaaagggaagccatcctgttatatcgcttggagaat
ttctcttttcggcatttggagttgctgaacttaacgtatacaaatgta
aacttttgatattatcaaatagcctgcttagagacctaactccaaaga
aatgtcagtttctggaaaagatacttcattcaccaaaaagtgtagtta
ctttgctttgtggagtgaagagttcagatcagctctatgaattactaa
atatctctcaaagcagatgggagatctcaactgaacaggaacctgaag
actacatctctgtaatccagagtatcatattcaaagattctgaagact
actttgaggtcaacattccaacagacctacgagcaaaacattctgggg
aaataagtgagagaaaggaaattgaagaactatcagaagatcaagaaa
caccataccactagcagtggtgcttcccactgaaattccatgtgagaa
tcctggtgaaatattcataattttgagagatgaagtaattggtgatac
tgtagaggttgaatttacatcaagtaataagcgcattagaacacggcc
agccattggaataagaaagtctggtgcatgaaagctttagagtttcct
gctggttcagtccatgtcaatgtctactgtgatggaatcgttaaagct
acaaccaaaattaagtactacccaacagcaaaggcaaaggaatgccta
ttcagaatggcagattcaggagagagtttgtgccagaatagcattgaa
gaacttgatggtgtccttacatccatattcaaacatgagataccatat
tatgagttccagtctcttcaaactgaaatttgttctcaaaacaaatat
actcatttcaaagaacttccaactcttctccactgtgcagcaaaattt
ggcttaaagaacctggctattcatttgcttcaatgttcaggagcaacc
tgggcatctaagatgaaaaatatggagggttcagaccccgcacatatt
gctgaaaggcatggtcacaaagaactcaagaaaatcttcgaagacttt
tcaatccaagaaattgacataaataatgagcaagaaaatgattatgaa
gaggatattgcctcattttccacatatattccttccacacagaaccca
gcatttcatcatgaaagcagaaagacatacgggcagagtgcagatgga
gctgaggcaaatgaaatggaaggggaaggaaaacagaatggatcaggc
atggagaccaaacacagcccactagaggttggcagtgagagttctgaa
gaccagtatgatgacttgtatgtgttcattcctggtgctgatccagaa
aataattcacaagagccactcatgagcagcagacctcctctccccccg
ccgcgacctgtagctaatgccttccaactggaaagacctcacttcacc
ttaccagggacaatggtggaaggccaaatggaaagaagtcaaaactgg
ggtcatcctggtgttagacaagaaacaggagatgaacccaaaggagaa
aaagagaagaaagaagaggaaaaagagcaggaggaggaagaagaccca
tatacttttgctgagattgatgacagtgaatatgacatgatattggcc
aatctgagtataaagaaaaaaactgggagtcggtctttcattataaat
agacctcctgcccccacaccccgacccacaagtatacctccaaaagag
gaaactacaccttacatagctcaagtgtttcaacaaaagacagccaga
agacaatctgatgatgacaagttccgtggtcttcctaagaaacaagac
agagctcggatagagagtccagccttttctactctcaggggctgtcta
actgatggtcaggaagaactcatcctcctgcaggagaaagtaaagaat
gggaaaatgtctatggatgaagctctggagaaatttaaacactggcag
atgggaaaaagtggcctggaaatgattcagcaggagaaattacgacaa
ctacgagactgcattattgggaaaaggccagaagaagaaaatgtctat
aataaactcaccattgtgcaccatccaggtggtaaggaaactgcccac
aatgaaaataagtttttataatgtacacttcagcaataagcttcctgc
tcgaccccaagttgaaaaggaatttggtttctgttgcaagaaagatca
ttaa
Gene symbol: CEBPG
Entrez Gene ID: 1054
dna/ac: BC007582.1
sequence:
(SEQ ID NO: 3)
atgagcaagatatcgcagcaaaacagcactccaggggtgaacggaatt
agtgttatccatacccaggcacatgccagcggcttacagcaggttcct
cagctggtgcctgctggccctgggggaggaggcaaagctgtggctccc
agcaagcagagcaaaaagagttcgcccatggatcgaaacagtgacgag
tatcggcaacgccgagagaggaacaacatggctgtgaaaaagagccgg
ttgaaaagcaagcagaaagcacaagacacactgcagagagtcaatcag
ctcaaagaagagaatgaacggttggaagcaaaaatcaaattgctgacc
aaggaattaagtgtactcaaagatttgtttcttgagcatgcacacaac
cttgcagacaacgtacagtccattagcactgaaaatacgacagcagat
ggcgacaatgcaggacagtag
Gene symbol: DDX55
Entrez Gene ID: 57696
dna/ac: BC030020.2
sequence:
(SEQ ID NO: 4)
atggagcatgtgacagagggctcctgggagtcgctgcctgtgccgctg
cacccgcaggtgctgggcgcgctgcgggagctgggcttcccgtacatg
acgccggtgcagtccgcaaccatccctctgttcatgcgaaacaaagat
gtcgctgcagaagcggtcacaggtagtggcaaaacactcgcttttgtc
atccccatcctggaaattcttctgagaagagaagagaagttaaaaaag
agtcaggttggagccataatcatcacccccactcgagagctggccatt
caaatagacgaggtcctgtcgcatttcacgaagcacttccccgagttc
agccagattctttggatcggaggcaggaatcctggagaagatgttgag
aggtttaagcaacaaggtgggaacatcattgtggccactccaggccgc
ttggaggacttgttccggaggaaggccgaaggcttggatctggccagc
tgtgtgcgatccctggatgtcctggtgttggatgaggcagacagactt
ctggacatggggtttgaggcaagcatcaacaccattctggagtttttg
ccaaagcagaggagaacaggccttttctctgccactcagacgcaggaa
gtggagaacctggtgagagcgggcctccggaaccctgtccgggtctca
gtgaaggagaagggcgtggcagccagcagtgcccagaagaccccctcc
cgcctggaaaactactacatggtatgcaaggcagatgagaaatttaat
cagctggtccattttcttcgcaatcataagcaggagaaacacctggtc
ttcttcagcacctgtgcctgtgtggaatactatgggaagactctggaa
gtgctggtgaagggcgtgaagattatgtgcattcacggaaagatgaaa
tataaacgcaataagatcttcatggagttccgcaaattgcaaagtggg
attttagtgtgcactgatgtgatggcccggggaattgatattcctgaa
gtcaactgggttttgcagtatgaccctcccagcaatgcaagtgccttc
gtgcatcgctgcggtcgcacagctcgcattggccacgggggcagcgct
ctggtgttcctectgcccatggaagagtcatacatcaatttccttgca
attaaccaaaaatgccccctgcaggagatgaagccccagagaaacaca
gcggaccttctgccaaaactcaagtccatggccctggctgacagagag
tgtttgaaaagggcatgaaagcttttgtgtcatatgtccaagcttatg
caaagcatgaatgcaacctgattttcagattaaaggatcttgattttg
ccagccttgctcgaggttttgccctgctgaggatgcccaagatgccag
aattgagagggaagcagtttccagattttgtgcccgtggacgttaata
ccgacacgattccatttaaagataaaatcagagaaaagcagaggcaga
aactcctggagcaacaaagaagagagaaaacagaaaatgaagggagaa
gaaaattcataaaaaataaagcttggtcaaagcagaaggccaaaaaag
aaaagaagaaaaaaatgaatgagaaaaggaaaagggaagagggttctg
atattgaagatgaggacatggaagaacttcttaatgacacaagactct
tgaaaaaacttaagaaaggcaaaataactgaagaagaatttgagaagg
gcttgttgacaactggcaaaagaacaatcaagacagtggatttaggga
tctcagatttggaagatggctgctaa
Gene symbol: DOM3Z
Entrez Gene ID: 1797
dna/ac: BC019083.2
sequence:
(SEQ ID NO: 5)
atggatcccagggggaccaagagaggagctgagaagacagaggtagct
gagcctcggaacaaactacctcgtccagcaccttctctgcccacagac
cctgccctotactctgggccctttcattctaccggcgcccttcggaac
tgggctgcttctccctggatgctcaacgccagtaccatggagatgccc
gagccctgcgctactatagcccaccccccactaacggtccaggcccca
actttgacctcagagacggatacccggatcgataccagccccgggacg
aggaggtccaggaaaggctggaccacctgctgtgctggctcctggaac
accgaggccggttggaggggggtccaggctggctggcagaggccatag
tgacgtggcgggggcacctgacaaaactgctgacgacaccgtatgagc
ggcaggagggctggcagctggcagcctcccggttccagggaacactat
acctgagtgaagtggagacaccgaacgctcgggcccagaggcttgacg
gccaccgctcctccgggagcttatgtacatgggatacaaatttgagca
gtacatgtgtgcagacaaacctggaagctccccagacccctctgggga
ggttaacaccaacgtggccttctgactgtgctacgcagccgcctggga
agccaccactgctcttctcaggggaggtagactgcacagacccccaag
ccccatccacacagcccccaacctgctatgtggagctcaagacctcca
aggagatgcacagccctggccaatggaggagtactacagacacaagct
cctgaaatggtgggctcagtcattcctcccaggggtcccgaatgttgt
tgctggcttccgtaacccagacggttttgtacttccctcaagaccttt
cctaccatgaagatgtttgaatatgtcaggaatgaccgtgacggctgg
aatccctctgtgtgcatgaacttctgtgccgccttccttagctttgcc
cagagcacggttgtccaggatgaccccaggctcgttcatctcttctct
tgggagcctggcggcccagtcaccgtgtctgtacaccaagatgcacct
tacgccttcctgcccatatggtatgtggaagctatgactcaggacctc
ccatcaccccccaagactccctctcccaaatag
Gene symbol: E1B-AP5
Entrez Gene ID: 11100
dna/ac: BC009988.2
sequence:
(SEQ ID NO: 6)
atggacaatattaccaggcagaaccaattctacgatacccaagtcatc
aaacaagaaaacgagtcaggctacgagaggagaccactggaaatggag
cagcagcaggcctatcgtccagaaatgaagacagagatgaagcaagga
gcacccaccagcttcctcccgcctgaagcttctcaactcaagccagac
aggcagcaattccagagtcgaaagaggccttatgaagaaaaccgggga
cgggggtactttgagcaccgagaggataggaggggccgctctcctcag
cctcctgctgaagaggatgaagatgactttgatgatacccttgttgct
attgacacctataactgcgacctccacttcaaggtggcccgagatcgg
agtagtggctatccgctcacaattgagggctttgcatacctgtggtca
ggagcccgtgccagctatggggtcagaaggggccgtgtatgcttcgag
atgaagatcaatgaggaaatctccgtgaagcaccttccgtctacagag
cctgacccccacgtggtccgtatcggctggtccctggactcctgcagc
acccagctaggcgaagagcctttctcctatggctatggaggcactggg
aagaagtccaccaatagccggtttgaaaactacggagacaagtttgca
gagaacgatgtgattggctgctttgcggattttgaatgtggaaatgac
gtggaactgtcttttaccaagaatggaaagtggatgggcattgctttc
cgaatccagaaggaagccttggggggtcaggccctctatcctcatgtc
ctggtgaagaattgcgcagtggagttcaacttcggacagagagcagag
ccctactgttctgtcctcccggggtttaccttcatccagcaccttccc
cttagtgagcgtatccggggcaccgttggaccaaagagcaaggcagaa
tgtgagattctgatgatggtgggcctgcctgctgctggcaagaccaca
tgggccatcaaacatgcagcctccaacccttccaagaagtacaacatc
ctgggtaccaatgccatcatggataagatgcgggtgatgggcctacgc
cggcagcggaactatgctggccgctgggatgtcctgatccagcaggcc
acccagtgcctcaaccgcctcatccagattgctgcccgcaagaaacgc
aactatatcctagatcagacaaatgtttatgggtcagcccagagacga
aaaatgagaccatttgaaggcttccagcgcaaagctattgtaatttgt
cccactgacgaggacctaaaagaccgaacaataaagcgaaccgacgag
gaagggaaggatgtcccagatcatgcggtcttagaaatgaaagccaac
ttcacgttgccagatgttggggacttcctggatgaggttctgttcatt
gagctgcagcgggaggaagcggacaagctagtgaggcagtacaacgag
gaaggccgcaaggctgggccaccccctgaaaagcgctttgacaaccga
ggtggtggtggcttccggggccgcgggggtggtggtggcttccagcgc
tatgaaaaccgaggaccccctggaggcaaccgtggcggcttccagaac
cgagggggaggcagcggtggaggaggcaactaccgaggaggtttcaac
cgcagcggaggtggtggctatagccagaaccgctggggtaacaacaac
cgggataacaacaactccaacaacagaggcagctacaaccgggctccc
cagcaacagccgccaccacagcagcctccgccaccacagccaccaccc
cagcagccaccgccaccacccagctacagccctgctcggaacccccca
ggggccagcacctacaataagaacagcaacatccctggctcaagcgcc
aataccagcacccccaccgtcagcagctacagccctccacagccgagt
tacagccagccaccctacaaccagggaggttacagccagggctacaca
gccccaccgcctccacctccaccaccacctgcctacaactatgggagc
tacggcggttacaacccggccccctataccccaccgccaccccccacc
gcacagacctaccctcagcccagctataaccagtatcagcagtatgcc
cagcagtggaaccagtactatcagaaccagggccagtggccgccatac
tacgggaactacgactacgggagctactccgggaacacacagggtggc
acaagtacacagtag
Gene symbol: FUS
Entrez Gene ID: 2521
dna/ac: BC000402.2
sequence:
(SEQ ID NO: 7)
atggcctcaaacgattatacccaacaagcaacccaaagctatggggcc
taccccacccagcccgggcagggctattcccagcagagcagtcagccc
tacggacagcagagttacagtggttatagccagtccacggacacttca
ggctatggccagagcagctattcttcttatggccagagccagaacaca
ggctatggaactcagtcaactccccagggatatggctcgactggcggc
tatggcagtagccagagctcccaatcgtcttacgggcagcagtcctcc
tatcctggctatggccagcagccagctcccagcagcacctcgggaagt
tacggtagcagttctcagagcagcagctatgggcagccccagagtggg
agctacagccagcagcctagctatggtggacagcagcaaagctatgga
cagcagcaaagctataatccccctcagggctatggacagcagaaccag
tacaacagcagcagtggtggtggaggtggaggtggaggtggaggtaac
tatggccaagatcaatcctccatgagtagtggtggtggcagtggtggc
ggttatggcaatcaagaccagagtggtggaggtggcagcggtggctat
ggacagcaggaccgtggaggccgcggcaggggtggcagtggtggcggc
ggcggcggcggcggtggtggttacaaccgcagcagtggtggctatgaa
cccagaggtcgtggaggtggccgtggaggcagaggtggcatgggcgga
agtgaccgtggtggcttcaataaatttggtggccctcgggaccaagga
tcacgtcatgactccgaacaggataattcagacaacaacaccatcttt
gtgcaaggcctgggtgagaatgttacaattgagtctgtggctgattac
ttcaagcagattggtattattaagacaaacaagaaaacgggacagccc
atgattaatttgtacacagacagggaaactggcaagctgaagggagag
gcaacggtctcttttgatgacccaccttcagctaaagcagctattgac
tggtttgatggtaaagaattctccggaaatcctatcaaggtctcatgg
ctactcgccgggcagactttaatcggggtggtggcaatggtcgtggag
gccgagggcgaggaggacccatgggccgtggaggctatggaggtggtg
gcagtggtggtggtggccgaggaggatttcccagtggaggtggtggcg
gtggaggacagcagcgagctggtgactggaagtgtcctaatcccacct
gtgagaatatgaacttctcttggaggaatgaatgcaaccagtgtaagg
cccctaaaccagatggcccaggagggggaccaggtggctctcacatgg
ggggtaactacggggatgatcgtcgtggtggcagaggaggctatgatc
gaggcggctaccggggccgcggcggggaccgtggaggcttccgagggg
gccggggtggtggggacagaggtggattggccctggcaagatggattc
caggggtgagcacagacaggatcgcagggagaggccgtattaa
Gene symbol: HAGH
Entrez Gene ID: 3029
dna/ac: BC000840.1
sequence:
(SEQ ID NO: 8)
atgaaggtagaggtgctgcctgccctgaccgacaactacatgtacctg
gtcattgatgatgagaccaaggaggctgccattgtggatccggtgcag
ccccagaaggtcgtggacgcggcgagaaagcacggggtgaaactgacc
acagtgctcaccacccaccaccactgggaccatgctggcgggaatgag
aaactggtcaagctggagtcgggactgaaggtgtacgggggtgacgac
cgtatcggggccctgactcacaagatcactcacctgtctacactgcag
gtggggtctctgaacgtcaagtgcctggcgaccccgtgccacacttca
ggacacatttgttacttcgtgagcaagcccggaggctcggagccccct
gccgtgttcacaggtgacaccttgtttgtggctggctgcgggaagttc
tatgaagggactgcggatgagatgtgtaaagctctgctggaggtcttg
ggccggctccccccggacacaagagtctactgtggccacgagtacacc
atcaacaacctcaagtttgcacgccacgtggagcccggcaatgccgcc
atccgggagaagctggcctgggccaaggagaagtacagcatcggggag
cccacagtgccatccaccctggcagaggagtttacctacaacccatca
tgagagtgagggagaagacggtgcagcagcacgcaggtgagacggacc
cggtgaccaccatgcgggccgtgcgcagggagaaggaccagttcaaga
tgccccgggactga
Gene symbol: HMG20B
Entrez Gene ID: 10362
dna/ac: BC002552.2
sequence:
(SEQ ID NO: 9)
atgtcccacggccccaagcagcccggcgcggccgccgcgccggcgggc
ggcaaggctccgggccagcatgggggcttcgtggtgactgtcaagcaa
gagcgcggcgagggtccacgcgcgggcgagaaggggtcccacgaggag
gagccggtgaagaaacgcggctggcccaagggcaagaagcggaagaag
attctgccgaatgggcccaaggcaccggtcacgggctacgtgcgcttc
ctgaacgagcggcgcgagcagatccgcacgcgccacccggatctgccc
tttcccgagatcaccaagatgctgggcgccgagtggagcaagctgcag
ccaacggaaaagcagcggtacctggatgaggccgagagagagaagcag
cagtacatgaaggagctgcgggcgtaccagcagtctgaagcctataag
atgtgcacggagaagatccaggagaagaagatcaagaaagaagactcg
agctctgggctcatgaacactctcctgaatggacacaagggtggggac
tgcgatggcttctccaccttcgatgttcccatcttcactgaagagtta
tggaccaaaacaaagcgcgtgaggeggagcttcggcgcttgcggaaga
tgaatgtggccttcgaggagcagaacgcggtactgcagaggcacacgc
agagcatgagcagcgcgcgcgagcgtctggagcaggagctggcgctgg
aggagcggaggacgctggcgctgcagcagcagctccaggccgtgcgcc
aggcgctcaccgccagettcgcctcactgccggtgccgggcacgggcg
aaacgcccacgctgggcactctggacttctacatggcccggatcacgg
agccatcgagcgcgaccccgcccagcacgagaagctcatcgtccgcat
caaggaaatcctggcccaggtcgccagcgagcacctgtga
Gene symbol: HOXB6
Entrez Gene ID: 3216
dna/ac: BC014651.1
sequence:
(SEQ ID NO: 10)
atgagttcctatttcgtgaactccaccttccccgtcactctggccagc
gggcaggagtccttcctgggccagctaccgctctattcgtcgggctat
gcggacccgctgagacattaccccgcgccctacgggccagggccgggc
caggacaagggctttgccacttcctcctattacccgccggcgggcggt
ggctacggccgagcggcgccctgcgactacgggccggcgccggccttc
taccgcgagaaagagtcggcctgcgcactctccggcgccgacgagcag
cccccgttccaccccgagccgcggaagtcggactgcgcgcaggacaag
agcgtgttcggcgagacagaagagcagaagtgctccactccggtctac
ccgtggatgcagcggatgaattcgtgcaacagttcctcctttgggccc
agcggccggcgaggccgccagacatacacacgttaccagacgctggag
ctggagaaggagtttcactacaatcgctacctgacgcggcggcggcgc
atcgagatcgcgcacgccctgtgcctgacggagaggcagatcaagata
tggttccagaaccgacgcatgaagtggaaaaaggagagcaaactgctc
agcgcgtctcagctcagtgccgaggaggaggaagaaaaacaggccgag
tga
Gene symbol: IFI16
Entrez Gene ID: 3428
dna/ac: BC017059.1
sequence:
(SEQ ID NO: 11)
atgggaaaaaaatacaagaacattgttctactaaaaggattagaggtc
atcaatgattatcatttttagaatggttaagtccttactgagcaacga
tttaaaacttaatttaaaaatgagagaagagtatgacaaaattcagat
tgctgacttgatggaagaaaagttccgaggtgatgctggtttgggcaa
actaataaaaattttcgaagatataccaacgcttgaagacctggctga
aactcttaaaaaagaaaagttaaaagtaaaaggaccagccctatcaag
aaagaggaagaaggaagtggatgctacttcacctgcaccctccacaag
cagcactgtcaaaactgaaggagcagaggcaactcctggagctcagaa
aagaaaaaaatcaaccaaagaaaaggctggacccaaagggagtaaggt
gtccgaggaacagactcagcctccctctcctgcaggagccggcatgtc
cacagccatgggccgttccccatctcccaagacctcattgtcagctcc
acccaacacttcttcaactgagaacccgaaaacagtggccaaatgtca
ggtaactcccagaagaaatgttctccaaaaacgcccagtgatagtgaa
ggtactgagtacaacaaagccatttgaatatgagaccccagaaatgga
gaaaaaaataatgtttcatgctacagtggctacacagacacagttctt
ccatgtgaaggttttaaacaccagcttgaaggagaaattcaatggaaa
gaaaatcatcatcatatcagattatttggaatatgatagtctcctaga
ggtcaatgaagaatctactgtatctgaagctggtcctaaccaaacgtt
tgaggttccaaataaaatcatcaacagagcaaaggaaactctgaagat
tgatattcttcacaaacaagcttcaggaaatattgtatatggggtatt
tatgctacataagaaaacagtaaatcagaagaccacaatctacgaaat
tcaggatgatagaggaaaaatggatgtagtggggacaggacaatgtca
caatatcccctgtgaagaaggagataagctccaacttttctgctttcg
acttagaaaaaagaaccagatgtcaaaactgatttcagaaatgcatag
ttttatccagataaagaaaaaaacaaacccgagaaacaatgaccccaa
gagcatgaagctaccccaggaacagagtcagcttccaaatccttcaga
ggccagcacaaccttccctgagagccatcttcggactcctcagatgcc
accaacaactccatccagcagtttcttcaccaagaaaagtgaagacac
aatctccaaaatgaatgacttcatgaggatgcagatactgaaggaagg
gagtcattttccaggaccgttcatgaccagcataggcccagctgagag
ccatccccacactcctcagatgcctccatcaacaccaagcagcagttt
cttaaccacgttgaaaccaagactgaagactgaacctgaagaagtttc
catagaagacagtgcccagagtgacctcaaagaagtgatggtgctgaa
cgcaacagaatcatttgtatatgagcccaaagagcagaagaaaatgtt
tcatgccacagtggcaactgagaatgaagtcttccgagtgaaggtttt
taatattgacctaaaggagaagttcaccccaaagaagatcattgccat
agcaaattatgtttgccgcaatgggttcctggaggtatatcctttcac
acttgtggctgatgtgaatgctgaccgaaacatggagatcccaaaagg
attgattagaagtgccagcgtaactcctaaaatcaatcagctttgctc
acaaactaaaggaagttttgtgaatggggtgtttgaggtacataagaa
aaatgtaaggggtgaattcacttattatgaaatacaagataatacagg
gaagatggaagtggtggtgcatggacgactgaccacaatcaactgtga
ggaaggagataaactgaaactcacctgctttgaattggcaccgaaaag
tgggaataccggggagttgagatctgtaattcatagtcacatcaaggt
catcaagaccaggaaaaacaagaaagacatactcaatcctgattcaag
tatggaaacttcaccagactttttcttctaa
Gene symbol: KRT8
Entrez Gene ID: 3856
dna/ac: BC008200.1
sequence:
(SEQ ID NO: 12)
atgaacaaggtagagctggagtctcgcctggaagggctgaccgacgag
atcaacttcctcaggcagctatatgaagaggagatccgggagctgcag
tcccagatctcggacacatctgtggtgctgtccatggacaacagccgc
tccctggacatggacagcatcattgctgaggtcaaggcacagtacgag
gatattgccaaccgcagccgggctgaggctgagagcatgtaccagatc
aagtatgaggagctgcagagcctggctgggaagcacggggatgacctg
cggcgcacaaagactgagatctctgagatgaaccggaacatcagccgg
ctccaggctgagattgagggcctcaaaggccagagggcttccctggag
gccgccattgcagatgccgagcagcgtggagagctggccattaaggat
gccaacgccaagttgtccgagctggaggccgccctgcagcgggccaag
caggacatggcgcggcagctgcgtgagtaccaggagctgatgaacgtc
aagctggccctggacatcgagatcgccacctacaggaagctgctggag
ggcgaggagagccggctggagtctgggatgcagaacatgagtattcat
acgaagaccaccagcggctatgcaggtggtctgagctcggcctatggg
ggcctcacaagccccggcctcagctacagcctgggctccagattggct
ctggcgcgggctccagctccttcagccgcaccagctcctccagggccg
tggttgtgaagaagatcgagacacgtgatgggaagctggtgtctgagt
cctctgacgtcctgcccaagtga
Gene symbol: LIN28
Entrez Gene ID: 79727
dna/ac: BCO28566.2
sequence:
(SEQ ID NO: 13)
atgggctccgtgtccaaccagcagtttgcaggtggctgcgccaaggcg
gcagaagaggcgcccgaggaggcgccggaggacgcggcccgggcggcg
gacgagcctcagctgctgcacggtgcgggcatctgtaagtggttcaac
gtgcgcatggggttcggcttcctgtccatgaccgcccgcgccggggtc
gcgctcgaccccccagtggatgtctttgtgcaccagagtaagctgcac
atggaagggttccggagcttgaaggagggtgaggcagtggagttcacc
tttaagaagtcagccaagggtctggaatccatccgtgtcaccggacct
ggtggagtattctgtattgggagtgagaggcggccaaaaggaaagagc
atgcagaagcgcagatcaaaaggagacaggtgctacaactgtggaggt
ctagatcatcatgccaaggaatgcaagctgccaccccagcccaagaag
tgccacttctgccagagcatcagccatatggtagcctcatgtccgctg
aaggcccagcagggccctagtgcacagggaaagccaacctactttcga
gaggaagaagaagaaatccacagccctaccctgctcccggaggcacag
aattga
Gene symbol: LNX
Entrez Gene ID: 84708
dna/ac: BCO22983.1
sequence:
(SEQ ID NO: 14)
atgaaccagccagagtctgccaacgatcctgaacccctgtgtgcagtg
tgtggccaagcccactccttggaggaaaaccacttctacagctatcca
gaggaagtggatgatgacctcatctgccacatctgcctgcaggattgc
tggacccectggacactccgtgtggacacacctactgcaccctctgcc
tcaccaacttcctggtggagaaggacttctgtcccatggaccgcaagc
ctctggttctgcagcactgcaagaagtccagcatcctggtcaacaaac
tcctcaacaagctactggtgacctgcccattcagggagcactgcaccc
aggtgttgcagcgctgtgacctcgagcatcactttcaaaccagctgta
aaggtgcctcccactacggcctgaccaaagataggaagaggcgctcac
aagatggctgtccagacggctgtgcgagcctcacagccacggctccct
ccccagaggtttctgcagctgccaccatctccttaatgacagacgagc
ctggcctagacaaccctgcctacgtgtcctcggcagaggacgggcagc
cagcaatcagcccagtggactctggccggagcaaccgaactagggcac
ggccctttgagagatccactattagaagcagatcatttaaaaaaataa
atcgagctttgagtgttcttcgaaggacaaagagcgggagtgcagttg
ccaaccatgccgaccagggcagggaaaattctgaaaacaccactgccc
ctgaagtctttccaaggttgtaccacctgattccagatggtgaaatta
ccagcatcaagatcaatcgagtagatcccagtgaaagcctctctatta
ggctggtgggaggtagcgaaaccccactggtccatatcattatccaac
acatttatcgtgatggggtgatcgccagagacggccggctactgccag
gagacatcattctaaaggtcaacgggatggacatcagcaatgtccctc
acaactacgctgtgcgtctcctgcggcagccctgccaggtgctgtggc
tgactgtgatgcgtgaacagaagttccgcagcaggaacaatggacagg
ccccggatgcctacagaccccgagatgacagctttcatgtgattctca
acaaaagtagccccgaggagcagcttggaataaaactggtgcgcaagg
tggatgagcctggggttttcatcttcaatgtgctggatggcggtgtgg
catatcgacatggtcagcttgaggagaatgaccgtgtgttagccatca
atggacatgatcttcgatatggcagcccagaaagtgcggctcatctga
ttcaggccagtgaaagacgtgttcacctcgtcgtgtcccgccaggttc
ggcagcggagccctgacatctttcaggaagccggctggaacagcaatg
gcagctggtccccagggccaggggagaggagcaacactcccaagcccc
tccatcctacaattacttgtcatgagaaggtggtaaatatccaaaaag
accccggtgaatctctcggcatgaccgtcgcagggggagcatcacata
gagaatgggatttgcctatctatgtcatcagtgttgagcccggaggag
tcataagcagagatggaagaataaaaacaggtgacattttgttgaatg
tggatggggtcgaactgacagaggtcagccggagtgaggcagtggcat
tattgaaaagaacatcatcctcgatagtactcaaagctttggaagtca
aagagtatgagccccaggaagactgcagcagcccagcagccctggact
ccaaccacaacatggccccacccagtgactggtccccatcctgggtca
tgtggctggaattaccacggtgcttgtataactgtaaagatattgtat
tacgaagaaacacagctggaagtctgggcttctgcattgtaggaggtt
atgaagaatacaatggaaacaaaccttttttcatcaaatccattgttg
aaggaacaccagcatacaatgatggaagaattagatgtggtgatattc
ttcttgctgtcaatggtagaagtacatcaggaatgatacatgcttgct
tggcaagactgctgaaagaacttaaaggaagaattactctaactattg
tttcttggcctggcacttttttatag
Gene symbol: MAP1S
Entrez Gene ID: 55201
dna/ac: BC006358.1
sequence:
(SEQ ID NO: 15)
atgggcgtgggccggctggacatgtatgtgctgcacccgccctccgcc
ggcgccgagcgcacgctggcctgtgtgtgcgccctgctggtgtggcac
cccgccggccccggcgagaaggtggtgcgcgtgctgttccccggttgc
accccgcccgcctgcctcctggacggcctggtccgcctgcagcacttg
aggttcctgcgagagcccgtggtgacgccccaggacctggaggggccg
gggcgagccgagagcaaagagagcgtgggctcccgggacagctcgaag
agagagggcctcctggccacccaccctagacctggccaggagcgccct
ggggtggcccgcaaggagccagcacgggctgaggccccacgcaagact
gagaaagaagccaagaccccccgggagttgaagaaagaccccaaaccg
agtgtctcccggacccagccgcgggaggtgcgccgggcagcctcttct
gtgcccaacctcaagaagacgaatgcccaggcggcacccaagccccgc
aaagcgcccagcacgtcccactctggcttcccgccggtggcaaatgga
ccccgcagcccgcccagcctccgatgtggagaagccagcccccccagt
gcggcctgcggctctccggcctcccagctggtggccacgcccagcctg
gagctggggccgatcccagccggggaggagaaggcactggagctgcct
ttggccgccagctcaatcccaaggccacgcacaccctcccctgagtcc
caccggagccccgcagagggcagcgagcggctgtcgctgagcccactg
cggggcggggaggccgggccagacgcctcacccacagtgaccacaccc
acggtgaccacgccctcactacccgcagaggtgggctccccgcactcg
accgaggtggacgagtccctgtcggtgtcctttgagcaggtgctgccg
ccatccgcccccaccagtgaggctgggctgagcctcccgctgcgtggc
ccccgggcgcggcgctcggcttccccacacgatgtggacctgtgcctg
gtgtcaccctgtgaatttgagcatcgcaaggcggtgccaatggcaccg
gcacctgcgtcccccggcagctcgaatgacagcagtgcccggtcacag
gaacgggcaggtgggctgggggccgaggagacgccacccacatcggtc
agcgagtccctgcccaccctgtctgactcggatcccgtgcccctggcc
cccggtgcggcagactcagacgaagacacagagggctttggagtccct
cgccacgaccctttgcctgaccccctcaaggtccccccaccactgcct
gacccatccagcatctgcatggtggaccccgagatgctgccccccaag
acagcacggcaaacggagaacgtcagccgcacccggaagcccctggcc
cgccccaactcacgcgctgccgcccccaaagccactccagtggctgct
gccaaaaccaaggggcttgctggtggggaccgtgccagccgaccactc
agtgcccggagtgagcccagtgagaagggaggccgggcacccctgtcc
agaaagtcctcaacccccaagactgccactcgaggcccgtcggggtca
gccagcagccggcccggggtgtcagccaccccacccaagtccccggtc
tacctggacctggcctacctgcccagcgggagcagcgcccacctggtg
gatgaggagttcttccagcgcgtgcgcgcgctctgctacgtcatcagt
ggccaggaccagcgcaaggaggaaggcatgcgggccgtcctggacgcg
ctactggccagcaagcagcattgggaccgtgacctgcaggtgaccctg
atccccactttcgactcggtggccatgcatacgtggtacgcagagacg
cacgcccggcaccaggcgctgggcatcacggtgttgggcagcaacagc
atggtgtccatgcaggatgacgccttcccggcctgcaaggtggagttc
tag
Gene symbol: NDUFV3
Entrez Gene ID: 4731
dna/ac: BCO21217.2
sequence:
(SEQ ID NO: 16)
atggctgccccgtgtttgctgcggcaaggacgagccggggcgctgaag
actatgaccaggaagcccaggtgtttcgaggacttgcttctacggttt
attgtctgcggaatcagggaagagtgaaaagggtcagccacagaattc
caagaagcaaagtccaccaaaaaatgtagtggaaccaaaggagagggg
caagacctagccacccagacagcagctgaattgtetaaaaacttatct
tcacccagttcttacccgccagctgtgaataagggcaggaaggtagct
agtcccagtcccagtggcagcgtgctattcacagatgaaggggttccg
aaatttttgtcaagaaagactttggtagagtttccacagaaagttctg
tctccattcagaaaacagggctctgattcagaagctcgtcaggtgggt
cggaaagtgacgtcgccttcgtcttcatcctcgtccagctcctctgat
tctgaatctgatgatgaggctgacgtttcagaggtcactcctcgagtg
gtgagcaaaggcagaggggggcttcgaaaaccagaggcctctcattcc
tttgaaaacagagccccccgagttacagtatcagcaaaagagaaaacc
ttgctgcagaagccgcatgtggacattactgatccagagaagccccac
cagccaaagaagaaagggtcccctgctaagccatcagaaggcagggaa
aatgcgagaccaaaaaccacaatgcccagatctcaagtagatgaagag
tttttgaagcaaagtttaaaggaaaaacaattgcagaaaacatttaga
ttaaatgaaatagataaagaaagccaaaagccatttgaagttaaagga
cccttacctgtccacacaaaatcagggttgtctgcgccaccgaagggc
agcccagcgcctgctgtgttggcagaagaggccagagcagaggggcag
ctgcaagccagtcctcctggggcggcagaggggcatctggaaaaaccc
gtgccagagccccagcgcaaggcggcccctcccctgcccagaaaggaa
acctcagggacgcagggaatagaaggccacctgaagggtggacaggca
atcgtggaagatcagataccaccaagcaatttggagacagttcctgtt
gagaataaccacggtttccatgaaaagacagcagcgctgaagcttgag
gccgagggcgaggccatggaagatgcagccgcgccagggaacgaccga
ggcggcacacaggagccagccccagtgcctgctgagccgtttgacaac
actacctacaagaacctgcagcatcatgactacagcacgtacaccttc
ttagacctcaacctcgaactctcaaaattcaggatgcctcagccctcc
tcaggccgggagtcacctcgacactga
Gene symbol: PABPC1
Entrez Gene ID: 26986
dna/ac: BC015958.2
sequence:
(SEQ ID NO: 17)
atgaaccccagtgcccccagctaccccatggcctcgctctacgtgggg
gacctccaccccgacgtgaccgaggcgatgctctacgagaagttcagc
ccggccgggcccatcctctccatccgggtctgcagggacatgatcacc
cgccgctccttgggctacgcgtatgtgaacttccagcagccggcggac
gcggagcgtgattggacaccatgaattttgatgttataaagggcaagc
cagtacgcatcatgtggtctcagcgtgatccatcacttcgcaaaagtg
gagtaggcaacatattcattaaaaatctggacaaatccattgataata
aagcactgtatgatacattttctgatttggtaacatcctttcatgtaa
ggtggtttgtgatgaaaatggttccaagggctatggatttgtacactt
tgagacgcaggaagcagctgaaagagctattgaaaaaatgaatggaat
gctcctaaatgatcgcaaagtatttgaggacgatttaagtctcgtaaa
gaacgagaagctgaacttggagctagggcaaaagaattcaccaatgtt
tacatcaagaattttggagaagacatggatgatgagcgccttaaggat
ctctttggcaagtttgggcctgccttaagtgtgaaagtaatgactgat
gaaagtggaaaatccaaaggatttggatttgtaagattgaaaggcatg
aagatgcacagaaagctgtggatgagatgaacggaaaggagctcaatg
gaaaacaaatttatgttggtcgagctcagaaaaaggtggaacggcaga
cggaacttaagcgcaaatttgaacagatgaaacaagataggatcacca
gataccagggtgttaatctttatgtgaaaaatcttgatgatggtattg
atgatgaacgtctccggaaagagttttctccatttggtacaatcacta
gtgcaaaggttatgatggagggtggtcgcagcaaagggtttggttttg
tatgtttacctccccagaagaagccactaaagcagttacagaaatgaa
cggtagaattgtggccacaaagccattgtatgtagctttagctcagcg
caaagaagagcgccaggctcacctcactaaccagtatatgcagagaat
ggcaagtgtacgagctgttcccaaccctgtaatcaacccctaccagcc
agcacctccttcaggttacttcatggcagctatcccacagactcagaa
ccgtgctgcatactatcctcctagccaaattgctcaactaagaccaag
tcctcgctggactgctcagggtgccagacctcatccattccaaaatat
gcccggtgctatccgcccagctgctcctagaccaccatttagtactat
gagaccagcttcttcacaggttccacgagtcatgtcaacacagcgtgt
tgctaacacatcaacacagacaatgggtccacgtectgcagctgcagc
cgctgcagctactcctgctgtccgcaccgttccacagtataaatatgc
tgcaggagttcgcaatcctcagcaacatcttaatgcacagccacaagt
tacaatgcaacagcctgctgttcatgtacaaggtcaggaacctttgac
tgcttccatgttggcatctgcccctcctcaagagcaaaagcaaatgtt
gggtgaacggctgtttcctcttattcaagccatgcaccctactcttgc
tggtaaaatcactggcatgttgttggagattgataattcagaacttct
tcatatgctcgagtctccagagtcactccgttctaaggttgatgaagc
tgtagctgtactacaagcccaccaagctaaagaggctgcccagaaagc
agttaacagtgccaccggtgttccaactgtttaa
Gene symbol: PHLDA1
Entrez Gene ID: 22822
dna/ac: BC018929.2
sequence:
(SEQ ID NO: 18)
atgctggagagtagcggctgcaaagcgctgaaggagggcgtgctggag
aagcgcagcgacgggttgttgcagctctggaagaaaaagtgttgcatc
ctcaccgaggaagggctgctgcttatcccgcccaagcagctgcaacac
cagcagcagcagcaacagcagcagcagcagcagcaacaacagcccggg
caggggccggccgagccgtcccaacccagtggccccgctgtcgccagc
ctcgagccgccggtcaagctcaaggaactgcacttctccaacatgaag
accgtggactgtgtggagcgcaagggcaagtacatgtacttcactgtg
gtgatggcagagggcaaggagatcgactttcggtgcccgcaagaccag
ggctggaacgccgagatcacgctgcagatggtgcagtacaagaatcgt
caggccatcctggcggtcaaatccacgcggcagaagcagcagcacctg
gtccagcagcagcccccctcgcagccgcagccgcagccgcagctccag
ccccaaccccagcctcagcctcagccgcaaccccagccccaatcacaa
ccccagcctcagccccaacccaagcctcagccccagcagctccacccg
tatccgcatccacatccacatccacactctcatcctcactcgcaccca
caccctcacccgcacccgcatccgcaccaaataccgcacccacaccca
cagccgcactcgcagccgcacgggcaccggcttctccgcagcacctcc
aactctgcctga
Gene symbol: PIAS2
Entrez Gene ID: 9063
dna/ac: BC015190.1
sequence:
(SEQ ID NO: 19)
atggcggatttcgaagagttgaggaatatggtttctagttttagggtt
tctgaactacaagtattactaggattgctggacggaataaaagtggac
gcaagcatgacctcctgatgagggcgctgcatttattgaagagcggct
gcagccctgcggttcagattaaaatccgagaattgtatagacgccgat
atccacgaactcttgaaggactttctgatttatccacaatcaaatcat
cggattcagtttggatggtggctcatcacctgtagaacctgacttggc
cgtggctggaatccactcgttgccttccacttcagttacacctcactc
accatcctctcctgttggttctgtgctgcttcaagatactaagcccac
atttgagatgcagcagccatctcccccaattcctcctgtccatcctga
tgtgcagttaaaaaatctgccatttatgatgtccttgatgactcatca
agcccacgagtttagttcaaagcagtattcagcgatttcaagagaagt
tttttatttttgctttgacacctcaacaagttagagagatatgcatat
ccagggattttttgccaggtggtaggagagattatacagtccaagttc
agttgagactttgcctggcagagacaagttgccctcaagaagataact
atccaaatagtctatgtataaaagtaaatgggaagctatttcctttgc
ctggctatgcaccaccgcctaaaaatgggattgaacagaagcgccctg
gacgccccttgaatattacatctttagttaggttatcttcagctgtgc
caaaccaaatttccatttcttgggcatcagaaattgggaagaattact
ctatgtctgtatatcttgtacggcagcttacatcagccatgttattac
agagattaaaaatgaaaggtattagaaaccctgatcattccagagcac
taattaaagaaaaacttactgcagatcctgatagtgaaattgctacaa
ctagccttcgggtatccttgatgtgccctttaggaaaaatgaggctga
caatcccatgccgtgcagtgacttgtacacatctgcagtgttttgatg
ctgccctctatctacaaatgaatgagaaaaagcccacctggatttgtc
ctgtgtgtgacaaaaaagctgcctatgaaagtctaatattagatgggc
tttttatggaaattctcaatgactgttctgatgtagatgagatcaaat
tccaagaagatggttcttggtgtccaatgagaccgaagaaagaagcta
tgaaagtatccagccaaccgtgtacaaaaatagaaagttcaagcgtcc
tcagtaagccttgttcagtgactgtagccagtgaggcaagcaagaaga
aagtagatgttattgatcttacaatagaaagctcttctgacgaagagg
aagaccctcctgccaaaaggaaatgcatctttatgtcagaaacacaaa
gcagcccaaccaaaggggttctcatgtatcagccatcttctgtaaggg
tgcccagtgtgacttcggttgatcctgctgctattccgccttcattaa
cagactactcagtaccattccaccatacgccaatatcaagcatgtcat
cagatttgccaggagaacaaagaagaaatgatattaataatgaaccga
agcttggaacatcttctgatactgtgcaacagtga
Gene symbol: PRKCBP1
Entrez Gene ID: 23613
dna/ac: BC030721.1
sequence:
(SEQ ID NO: 20)
atgacaatgctcaccattgaacagttatcctacctgctcaagtttgcc
attcagaaaatgaaacagccagggacagatgcattccagaagcccgtt
ccattggaacagcaccctgactatgcggaatacatcttccatccaatg
gacctttgtacattggaaaagaatgcgaaaaagaaaatgtatggctgc
acagaagccttcctggctgatgcaaagtggattttgcacaactgcatc
atttataatgggggaaatcacaaattgacgcaaatagcgaaagtagtc
atcaaaatctgtgaacatgagatgaatgaaatcgaagtatgtccagaa
tgttatctagctgcttgccaaaaacgagataactggttttgtgagcct
tgtagcaatccacatcctttggtctgggccaaactgaaggggtttcca
ttctggcctgcaaaagctctaagggataaagacgggcaggtcgatgcc
cgattctttggacaacatgacagggcctgggttccaataaataattgc
tacctcatgtctaaagaaattcctttttctgtgaaaaagactaagagc
atcttcaacagtgccatgcaagagatggaggtttacgtggagaacatc
cgcaggaagtttggggtttttaattactctccatttaggacaccctac
acacccaacagccagtatcaaatgctgctcgatcccaccaaccccagc
gccggcactgccaagatagacaagcaggagaaggtcaagctcaacttt
gacatgacggcatcccccaagatcctgatgagcaagcctgtgctgagt
gggggcacaggccgccggatttccttgtcggatatgccgcgctccccc
atgagcacaaactcttctgtgcacacgggctccgacgtggagcaggat
gctgagaagaaggccacgtcgagccacttcagtgcgagcgaggagtcc
atggacttcctggataagagcacagcttcaccagcctccaccaagacg
ggacaagcagggagtttatccggcagcccaaagcccttctctcctcaa
ctgtcagctcctatcacgacgaaaacggacaaaacctccaccaccggc
agcatcctgaatcttaacctggatcgaagcaaagctgagatggatttg
aaggagctgagcgagtcggtccagcaacagtccacccctgttcctctc
atctctcccaagcgccagattcgtagcaggttccagctgaatcttgac
aagaccatagagagttgcaaagcacaattaggcataaatgaaatctcg
gaagatgtctatacggccgtagagcacagcgattcggaggattctgag
aagtcagatagtagcgatagtgagtatatcagtgatgatgagcagaag
tctaagaacgagccagaagacacagaggacaaagaaggttgtcagatg
gacaaagagccatctgctgttaaaaaaaagcccaagcctacaaaccca
gtggagattaaagaggagctgaaaagcacgtcaccagccagcgagaag
gcagaccctggagcagtcaaggacaaggccagccctgagcctgagaag
gacttttccgaaaaggcaaaaccttcacctcaccccataaaggataaa
ctgaagggaaaagatgagacggattccccaacagtccatttgggcctg
gactctgattcagagagcgaacttgtcatagatttaggagaagaccat
tctgggcgggagggtcgaaaaaataagaaggaacccaaagaaccatct
cccaaacaggatgttgtaggtaaaactccaccatccacgacggtgggc
agccattctcccccggaaacaccggtgctcacccgctcttccgcccaa
acttccgcggctggcgccacagccaccaccagcacgtcctccacggtc
accgtcacggccccggcccccgccgccacaggaagcccagtgaaaaag
cagaggccgcttttaccgaaggagactgccccggccgtgcagcgggtc
gtgtggaactcatcaactgtccagcagaaggagatcacacagagccca
tccacgtccaccatcaccctggtgaccagcacacagtcatcgcccctg
gtcaccagctcggggtccatgagcacccttgtgtcctcagtcaacgct
gacctgcccatcgccactgcctcagctgatgtcgccgctgatattgcc
aagtacactagcaaaatgatggatgcaataaaaggaacaatgacagaa
atatacaacgatctttctaaaaacactactggaagcacaatagctgag
attcgcaggctgaggatcgagatagagaagctccagtggctgcaccag
caagagctctccgaaatgaaacacaacttagagctgaccatggcggag
atgcggcagagcctggagcaggagcgggaccggctcatcgccgaggtg
aagaagcagctggagttggagaagcagcaggcggtggatgagaccaag
aagaagcagtggtgcgccaactgcaagaaggaggccatcttttactgc
tgttggaacaccagctactgtgactacccctgccagcaagcccactgg
cctgagcacatgaagtcctgcacccagtcagctactgctcctcagcag
gaagcggatgctgaggtgaacacagaaacactaaataagtcctcccag
gggagctcctcgagcacacaatcagcaccttcagaaacggccagcgcc
tccaaagagaaggagacgtcagctgagaaaagcaaggagagtggctcg
acccttgacctttctggctccagagagacgccctcctccattctctta
ggctccaaccaaggctctgttagcaaaaggtgtgacaagcaacctgcc
tatgccccaaccaccacagaccaccagccgcaccccaactaccccgcc
cagaagtaccattcccggagtaataaatccagttggagcagcagtgat
gagaagaggggatcgacacgttccgatcacaacaccagtaccagcacg
aagagcctcctcccgaaagagtctcggctggacaccttctgggactag
Gene symbol: PRKRA
Entrez Gene ID: 8575
dna/ac: BC009470.1
sequence:
(SEQ ID NO: 21)
atgtcccagagcaggcaccgcgccgaggccccgccgctggagcgcgag
gacagtgggaccttcagtttggggaagatgataacagctaagccaggg
aaaacaccgattcaggtattacacgaatacggcatgaagaccaagaac
atcccagtttatgaatgtgaaagatctgatgtgcaaatacacgtgccc
actttcaccttcagagtaaccgttggtgacataacctgcacaggtgaa
ggtacaagtaagaagctggcgaaacatagagctgcagaggctgccata
aacattttgaaagccaatgcaagtatttgctttgcagttcctgacccc
ttaatgcctgacccttccaagcaaccaaagaaccagcttaatcctatt
ggttcattacaggaattggctattcatcatggctggagacttcctgaa
tataccctttcccaggagggaggacctgctcataagagagaatatact
acaatttgcaggctagagtcatttatggaaactggaaagggggcatca
aaaaagcaagccaaaaggaatgctgctgagaaatttcttgccaaattt
agtaatatttctccagagaaccacatttctttaacaaatgtagtagga
cattctttaggatgtacttggcattccttgaggaattctcctggtgaa
aagatcaacttactgaaaagaagcctccttagtattccaaatacagat
tacatccagctgcttagtgaaattgccaaggaacaagttttaatataa
catatttggatatagatgaactgagcgccaatggacaatatcaatgtc
ttgctgaactgtccaccagccccatcacagtctgtcatggctccggta
tctcctgtggcaatgcacaaagtgatgcagctcacaatgctttgcagt
atttaaagataatagcagaaagaaagtaa
Gene symbol: PSME3
Entrez Gene ID: 10197
dna/ac: BC001423.2
sequence:
(SEQ ID NO: 22)
atggcctcgttgctgaaggtggatcaggaagtgaagctcaaggttgat
tctttcagggagcggatcacaagtgaggcagaagacttggtggcaaat
tttttcccaaagaagttattagaacttgatagttttctgaaggaacca
atcttaaacatccatgacctaactcagatccactctgacatgaatctc
ccagtccctgaccccattcttctcaccaatagccatgatggactggat
ggtcccacttataagaagcgaaggttggatgagtgtgaagaagccttc
caaggaaccaaggtgtttgtgatgcccaatgggatgctgaaaagcaac
cagcagctggtggacattattgagaaagtgaaacctgagatccggctg
ttgattgagaaatgtaacacggtcaaaatgtgggtacagctcctgatt
cccaggatagaagatggaaacaactttggggtgtccattcaggaggaa
acagttgcagagctaagaactgttgagagtgaagctgcatcttatctg
gaccagatttctagatattatattacaagagccaaattggtttctaaa
atagctaaatatccccatgtggaggactatcgccgcaccgtgacagag
attgatgagaaagaatatatcagccttcggctcatcatatcagagctg
aggaatcaatatgtcactctacatgacatgatcctgaaaaatatcgag
aagatcaaacggccccggagcagcaatgcagagactctgtactga
Gene symbol: RAB11FIP3
Entrez Gene ID: 9727
dna/ac: BC051360.1
sequence:
(SEQ ID NO: 23)
atggcgtcggccccgccggcctcgcccccgggctcggagccgccgggg
cccgacccggagccgggcgggccggacgggccgggggcggcgcaactg
gctccgggccctgcggagctacgcctcggagcgcccgtcggcggcccc
gacccgcagtccccgggcctggatgagcctgcgcccggggccgctgca
gatggcggggcgcgttggagcgccgggccggccccggggctggaggga
ggcccgcgagaccccgggccgtccgccccgccgccgcgctccggcccg
cgggggcagcttgcgagccccgacgccccgggcccagggccgcgctcc
gaagcgccgcttccagaactcgacccgttgttctcctggactgaggag
cccgaggagtgtggccccgcgagctgcccggagagcgcgcctttccgc
ttgcaggggtccagcagcagccaccgagcgcggggcgaggtcgacgtc
ttctctcccttccccgcgcccacggcgggcgagctggcgctggagcaa
ggtcccgggtccccgccgcagccctcggacctcagccagacccacccc
cttccgagcgagcccgtggggagtcaggaggacggcccccgcctccga
gccgtgttcgatgccctggacggggatggggacggtttcgtccgcatc
gaggacttcatccagtttgctacggtctacggggcagagcaggtgaag
gacttaactaagtacttggatcccagtgggctcggcgtgatcagcttt
gaagacttctaccaagggatcacagccatcagaaacggagatcctgat
ggccagtgctacggtggtgtcgcttctgcccaagatgaggagcccctg
gcctgcccggacgagttcgatgacttcgtcacctatgaggccaacgag
gtgacggacagcgcgtacatgggctccgagagcacctacagtgagtgt
gagaccttcacggacgaggacaccagcaccctggtgcaccctgagctg
caacctgaaggggacgcagacagtgccggcggctcggccgtgccctct
gagtgcctggacgccatggaggagcccgaccatggtgccctgctgctg
ctcccaggcaggcctcacccccatggccagtctgtcatcacggtgatc
gggggcgaggagcactttgaggactacggtgaaggcagtgaggcggag
ctgtccccagagaccctatgcaacgggcagctgggctgcagtgacccc
gctttcctcacgcccagtccgacaaagcggctctccagcaagaaggtg
gcaaggtacctgcaccagtcaggggccctgaccatggaggccctggag
gacccttcccccgagctcatggagggcccagaggaggacattgctgac
aaggttgtcttcctggaaaggcgtgtgctggagctggaaaaggacacg
gcagccaccggtgagcaacacagccgcctgaggcaggagaacctgcag
ctggtgcacagagcaaacgccctggaggagcagctgaaggagcaggag
ctgagagcctgcgagatggtcctggaagagacccggcgtcagaaggag
ctcctgtgcaagatggagagggagaagagcattgagatcgagaacctg
cagaccaggctacagcaactggacgaggagaacagtgaactccggtcc
tgcacgccctgtctgaaggccaacattgagcgtctggaggaggagaag
cagaagctgttggatgagatagagtcgctgacgctgcggctcagtgaa
gagcaggagaacaagaggagaatgggggacaggctgagtcacgagagg
caccagttccagagggacaaggaggccacccaggagctgatcgaggac
ctccgaaagcagctggagcacctgcagctcctcaagctggaggccgag
cagcggcggggccgcagcagcagcatgggcctgcaggagtaccacagc
cgcgcccgggagagcgagctggagcaggaggtccgcaggctgaagcag
gacaaccgcaacctgaaggagcagaacgaggagctgaacgggcagatc
attaccctcagcatccagggcgccaagagcctcttctccacagccttc
tctgagtccctggctgcagagatcagctccgtctcccgagatgagctc
atggaggcgattcagaagcaggaggagatcaacttccgcctgcaggac
tacatcgacaggatcatcgtggccatcatggagaccaacccgtccatc
ctggaggtcaagtag
Gene symbol: RALBP1
Entrez Gene ID: 10928
dna/ac: BC013126.1
sequence:
(SEQ ID NO: 24)
atgactgagtgcttcctgccccccaccagcagccccagtgaacaccgc
agggtggagcatggcagcgggcttacccggacccccagctctgaagag
atcagccctactaagtttcctggattgtaccgcactggcgagccctca
cctccccatgacatcctccatgagcctcctgatgtagtgtctgatgat
gagaaagatcatgggaagaaaaaagggaaatttaagaaaaaggaaaag
aggactgaaggctatgcagcctttcaggaagatagctctggagatgag
gcagaaagtccttctaaaatgaagaggtccaagggaatccatgttttc
aagaagcccagcttttctaaaaagaaggaaaaggattttaaaataaaa
gagaaacccaaagaagaaaagcataaagaagaaaagcacaaagaagaa
aaacataaagagaagaagtcaaaagacttgacagcagctgatgttgtt
aaacagtggaaggaaaagaagaaaaagaaaaagccaattcaggagcca
gaggtgcctcagattgatgttccaaatctcaaacccatttttggaatt
cattggctgatgcagtagagaggaccatgatgtatgatggcattcggc
tgccagccgttttccgtgaatgtatagattacgtagagaagtatggca
tgaagtgtgaaggcatctacagagtatcaggaattaaatcaaaggtgg
atgagctaaaagcagcctatgaccgggaggagtctacaaacttggaag
actatgagcctaacactgtagccagtttgctgaagcagtatttgcgag
accttccagagaatttgataccaaagagcttatgcccagatttgaaga
ggcttgtgggaggaccacggagactgagaaagtgcaggaattccagcg
tttactcaaagaactgccagaatgtaactatcttctgatttatggctc
attgtgcacatggaccatgtcattgcaaaggaactggaaacaaaaatg
aatatacagaacatttctatagtgctcagcccaactgtgcagatcagc
aatcgagtcctgtatgtgtttttcacacatgtgcaagaactattggaa
atgtggtactaaagcaagtgatgaaacctctgcgatggtctaacatgg
ccacgatgcccacgctgccagagacccaggcgggcatcaaggaggaga
tcaggagacaggagtttatttgaattgtttacatcgagatctgcaggg
tgggataaaggatttgtctaaagaagaaagattatgggaagtacaaag
aattttgacagccctcaaaagaaaactgagagaagctaaaagacagga
gtgtgaaaccaagattgcacaagagatagccagtattcaaaagaggat
gatccaaagaagagatgaatgaaaatgaagaagttataaatattctcc
ttgctcaggagaatgagatcctgactgaacaggaggagctcctggcca
tggagcagtttctgcgccggcagattgcctcagaaaaagaagagattg
aacgcctcagagctgagattgctgaaattcagagtcgccagcagcacg
gccgaagtgagactgaggagtactcctccgagagcgagagcgagagtg
aggatgaggaggagctgcagatcattctggaagacttacagagacaga
acgaagagctggaaataaagaacaatcatttgaatcaagcaattcatg
aggagcgcgaggccatcatcgagctgcgcgtgcagctgcggctgctcc
agatgcagcgagccaaggccgagcagcaggcgcaggaggacgaggagc
ctgagtggcgcgggggtgccgtccagccgcccagagacggcgtccttg
agccaaaagcagctaaagagcagccaaaggcaggcaaggagccggcaa
agccatcgcccagcagggataggaaggagacgtccatctga
Gene symbol: RAN
Entrez Gene ID: 5901
dna/ac: BC014518.2
sequence:
(SEQ ID NO: 25)
atggctgcgcagggagagccccaggtccagttcaaacttgtattggtt
ggtgatggtggtactggaaaaacgaccttcgtgaaacgtcatttgact
ggtgaatttgagaagaagtatgtagccaccttgggtgttgaggttcat
cccctagtgttccacaccaacagaggacctattaagttcaatgtatgg
gacacagccggccaggagaaattcggtggactgagagatggctattat
atccaagcccagtgtgccatcataatgtttgatgtaacatcgagagtt
acttacaagaatgtgcctaactggcatagagatctggtacgagtgtgt
gaaaacatccccattgtgttgtgtggcaacaaagtggatattaaggac
aggaaagtgaaggcgaaatccattgtcttccaccgaaagaagaatctt
cagtactacgacatttctgccaaaagtaactacaactttgaaaagcca
tcctctggcttgctaggaagctcattggagaccctaacttggaatttg
ttgccatgcctgctctcgccccaccagaagttgtcatggacccagctt
tggcagcacagtatgagcacgacttagaggttgctcagacaactgctc
tcccggatgaggatgatgacctgtga
Gene symbol: RARA
Entrez Gene ID: 5914
dna/ac: BC008727.2
sequence:
(SEQ ID NO: 26)
atggccagcaacagcagctcctgcccgacacctgggggcgggcacctc
aatgggtacccggtgcctccctacgccttcttcttcccccctatgctg
ggtggactctccccgccaggcgctctgaccactctccagcaccagctt
ccagttagtggatatagcacaccatccccagccaccattgagacccag
agcagcagttctgaagagatagtgcccagccctccctcgccaccccct
ctaccccgcatctacaagccttgctttgtctgtcaggacaagtcctca
ggctaccactatggggtcagcgcctgtgagggctgcaagggcttcttc
cgccgcagcatccagaagaacatggtgtacacgtgtcaccgggacaag
aactgcatcatcaacaaggtgacccggaaccgctgccagtactgccga
ctgcagaagtgctttgaagtgggcatgtccaaggagtctgtgagaaac
gaccgaaacaagaagaagaaggaggtgcccaagcccgagtgctctgag
agctacacgctgacgccggaggtgggggagctcattgagaaggtgcgc
aaagcgcaccaggaaaccttccctgccctctgccagctgggcaaatac
actacgaacaacagctcagaacaacgtgtctctctggacattgacctc
tgggacaagttcagtgaactctccaccaagtgcatcattaagactgtg
gagttcgccaagcagctgcccggcttcaccaccctcaccatcgccgac
cagatcaccctcctcaaggctgcctgcctggacatcctgatcctgcgg
atctgcacgcggtacacgcccgagcaggacaccatgaccttctcggac
gggctgaccctgaaccggacccagatgcacaacgctggcttcggcccc
ctcaccgacctggtctttgccttcgccaaccagctgctgcccctggag
atggatgatgcggagacggggctgctcagcgccatctgcctcatctgc
ggagaccgccaggacctggagcagccggaccgggtggacatgctgcag
gagccgctgctggaggcgctaaaggtctacgtgcggaagcggaggccc
agccgcccccacatgttccccaagatgctaatgaagattactgacctg
cgaagcatcagcgccaagggggctgagcgggtgatcacgctgaagatg
gagatcccgggctccatgccgcctctcatccaggaaatgttggagaac
tcagagggcctggacactctgagcggacagccggggggtggggggcgg
gacgggggtggcctggcccccccgccaggcagctgtagccccagcctc
agccccagctccaacagaagcagcccggccacccactccccgtga
Gene symbol: RBMS1
Entrez Gene ID: 5937
dna/ac: BC012992.2
sequence:
(SEQ ID NO: 27)
atgggcaaagtgtggaaacagcagatgtaccctcagtacgccacctac
tattacccccagtatctgcaagccaagcagtctctggtcccagcccac
cccatggcccctcccagtcccagcaccaccagcagtaataacaacagt
agcagcagtagcaactcaggatgggatcagctcagcaaaacgaacctc
tatatccgaggactgcctccccacaccaccgaccaggacctggtgaag
ctctgtcaaccatatgggaaaatagtctccacaaaggcaattttggat
aagacaacaaacaaatgcaaaggttatggttttgtcgactttgacagc
cctgcagcagctcaaaaagctgtgtctgccctgaaggccagtggggtt
caagctcaaatggcaaagcaacaggaacaagatcctaccaacctctac
atttctaatttgccactctccatggatgagcaagaactagaaaatatg
ctcaaaccatttggacaagttatttctacaaggatactacgtgattcc
agtggtacaagtcgtggtgttggctttgctaggatggaatcaacagaa
aaatgtgaagctgttattggtcattttaatggaaaatttattaagaca
ccaccaggagtttctgcccccacagaacctttattgtgtaagtttgct
gatggaggacagaaaaagagacagaacccaaacaaatacatccctaat
ggaagaccatggcatagagaaggagaggctggaatgacacttacttac
gacccaactacagctgctatacagaacggattttatccttcaccatac
agtattgctacaaaccgaatgatcactcaaacttctattacaccctat
attgcatctcctgtatctgcctaccaggtgcaaagtccttcgtggatg
caacctcaaccatatattctacagcaccctggtgccgtgttaactccc
tcaatggagcacaccatgtcactacagcccgcatcaatgatcagccct
ctggcccagcagatgagtcatctgtcactaggcagcaccggaacatac
atgcctgcaacgtcagctatgcaaggagcctacttgccacagtatgca
catatgcagacgacagcggttcclgttgaggaggcaagtggtcaacag
caggtggctgtcgagacgtctaatgaccattctccatatacctttcaa
cctaataagtaa
Gene symbol: RDBP
Entrez Gene ID: 7936
dna/ac: BC050617.2
sequence:
(SEQ ID NO: 28)
atgttggtgataccccccggactgagcgaggaagaggaggctctgcag
aagaaattcaacaagctcaagaaaaagaaaaaggcattgctggctctg
aagaagcaaagtagcagcagcacaaccagccaaggtggtgtcaaacgc
tcactatcagagcagcctgtcatggacacagccacagcaacagagcag
gcaaagcagctggtgaagtcaggagccatcagtgccatcaaggctgag
accaagaactcaggcttcaagcgttctcgaacccttgaggggaagtta
aaggaccccgagaagggaccagtccccactttccagccgttccagagg
agcatatctgctgatgatgacctgcaagagtcatccagacgtccccag
aggaaatctctgtatgagagctttgtgtcttctagtgatcgacttcga
gaactaggaccagatggagaagaggcagagggcccaggggctggtgat
ggtccccctcgaagctttgactggggctatgaagaacgcagtggtgcc
cactcctcagcctcccctccccgaagccgcagccgggaccgcagccat
gagaggaaccgggacagagaccgagatcgggagcgggatcgagaccgg
gatcgagacagagacagagagcgggacagggatcgggatcgggatcga
gatcgagaccgggaacgggacagggatcgggagcgggatcgagaccga
gaccgagagggtcctttccgcaggtcggattcattccctgaacggcga
gcccctaggaaagggaatactctctatgtatatggagaagacatgaca
cccacccttctccgtggggccttctctccttttggaaacatcattgac
ctctccatggacccacccagaaactgtgccttcgtcacctatgaaaag
atggagtcagcagatcaggccgttgctgagctcaacgggacccaggtg
gagtctgtacagctcaaagtcaacatagcccgaaaacagcccatgctg
gatgccgctactggcaagtctgtctggggctccctcgctgtccagaac
agccctaagggttgccaccgggacaagaggacccagattgtctacagt
gatgacgtctacaaggaaaaccttgtggatggcttctag
Gene symbol: RNF12
Entrez Gene ID: 51132
dna/ac: BC013357.2
sequence:
(SEQ ID NO: 29)
atggaaaactcagattccaatgacaaaggaagtggtgatcagtctgca
gcacagcgcagaagtcagatggaccgattggatcgagaagaagctttc
tatcaatttgtaaataacctgagtgaagaagattataggcttatgaga
gataacaatttgctaggcaccccaggtgaaagtactgaggaagagttg
ctgagacgactacagcaaattaaagaaggcccaccaccgcaaaactca
gatgaaaatagaggaggagactcttcagatgatgtgtctaatggtgac
tctataatagactggcttaactctgtcagacaaactggaaatacaaca
agaagtgggcaaagaggaaaccaatcttggagagcagtgagtcggact
aatccaaacagtggtgatttcagattcagtttagagataaatgttaac
cgtaataatgggagccaaaattcagagaatgaaaatgagccatctgca
agacgttctagtggagaaaatgtggaaaacaacagccaaaggcaagtg
gaaaacccacgatctgaatcaacatctgcaaggccatctagatcagaa
cgaaattcaactgaagcgttaacagaggtcccacctaccagaggtcag
aggagggcaagaagcaggagcccagaccatcggagaaccagagcaaga
gctgaaagaagtaggtcacctctgcatccaatgagtgaaattccacga
agatctcatcatagtatctcatctcagacttttgaacatcctttggta
aatgagacggagggaagttctagaacccggcaccatgtgacattgagg
cagcaaatatctgggcctgagttgctaagtagaggtctttttgcagct
tctggaacaagaaatgcttctcaaggagcaggttcttcagacacagct
gccagtggtgaatctacaggatcaggacagagacctccaaccatagtc
cttgatcttcaagtaagaagagttcgtcctggagaatatcggcagaga
gatagcatagccagcagaactcggtctaggtctcagacaccaaacaac
actgtcacctatgaaagtgaacgaggaggttttaggcgtacattttca
cgttctgagcgggcaggtgtgagaacctatgtcagtaccatcagaatt
cccattcgtagaatcttaaatactggtttaagtgagactacatctgtt
gcaattcagaccatgttaaggcagataatgacaggttttggtgagtta
agctattttatgtacagtgatagcgactcagagcctactggctcagtc
tcaaatcgaaatatggaaagggcagagtcacggagtggaagaggaggt
tctggtggtggtagtagttctggttccagttcgagttccagttccagt
tcgagttccagttccagttcaagttccagttccagtcctagttccagt
tccggtggtgaaagttcagaaactagctcagatttatttgaaggcagt
aatgaaggaagctcatcatcaggctcatcaggtgccaggcgagagggt
cgacatagggccccagtcacatttgatgaaagtggctctttgcccttc
cttagcctggctcagtttttcctcttaaatgaggatgatgatgaccaa
cctagaggactcaccaaagaacagattgacaacttggcaatgagaagt
tttggtgaaaatgatgcattaaaaacctgtagtgtttgcattacagaa
tatacagaaggcaacaaacttcgtaaactaccttgttcccatgagtac
catgtccactgcatcgatcgctggttatctgagaattctacctgtcct
atttgtcgcagagcagtcttagcttctggtaacagagaaagtgttgtg
taa
Gene symbol: RPL30
Entrez Gene ID: 6156
dna/ac: BC032700.2
sequence:
(SEQ ID NO: 30)
atggtggccgcaaagaagacgaaaaagtcgctggagtcgatcaactct
aggctccaactcgttatgaaaagtgggaagtacgtcctggggtacaag
cagactctgaagatgatcagacaaggcaaagcgaaattggtcattctc
gctaacaactgcccagctttgaggaaatctgaaatagagtactatgct
atgttggctaaaactggtgtccatcactacagtggcaataatattggg
cacagcatgcggaaaatactacagagtgtgcacactggctatcattga
tccaggtgactctgacatcattagaagcatgccagaacagactggtga
aaagtaa
Gene symbol: RPL31
Entrez Gene ID: 6160
dna/ac: BC017343.2
sequence:
(SEQ ID NO: 31)
atggctcccgcaaagaagggtggcgagaagaaaaagggccgttctgcc
atcaacgaagtggtaacccgagaatacaccatcaacattcacaagcgc
atccatggagtgggcttcaagaagcgtgcacctcgggcactcaaagag
attcggaaatttgccatgaaggagatgggaactccagatgtgcgcatt
gacaccaggctcaacaaagctgtctgggccaaaggaataaggaatgtg
ccataccgaatccgtgtgcggctgtccagaaaacgtaatgaggatgaa
gattcaccaaataagctatatactttggttacctatgtacctgttacc
actttcaaaaatctacagacagtcaatgtggatgagaactaa
Gene symbol: RUFY1
Entrez Gene ID: 80230
dna/ac: BC032571.1
sequence:
(SEQ ID NO: 32)
atgatggaggagcgtgccaacctgatgcacatgatgaaactcagcatc
aaggtgttgctccagtcggctctgagcctgggccgcagcctggatgcg
gaccatgcccccttgcagcagttctttgtagtgatggagcactgcctc
aaacatgggctgaaagttaagaagagttttattggccaaaataaatca
ttctttggtcctttggagctggtggagaaactttgtccagaagcatca
gatatagcgactagtgtcagaaatcttccagaattaaagacagctgtg
ggaagaggccgagcgtggctttatcttgcactcatgcaaaagaaactg
gcagattatctgaaagtgcttatagacaataaacatctcttaagcgag
ttctatgagcctgaggctttaatgatggaggaagaagggatggtgatt
gttggtctgctggtgggactcaatgttctcgatgccaatctctgcttg
aaaggagaagacttggattctcaggttggagtaatagatttttccctc
taccttaaggatgtgcaggatcttgatggtggcaaggagcatgaaaga
attactgatgtccttgatcaaaaaaattatgtggaagaacttaaccgg
cacttgagctgcacagttggggatcttcaaaccaagatagatggcttg
gaaaagactaactcaaagcttcaagaagagctttcagctgcaacagac
cgaatttgctcacttcaagaagaacagcagcagttaagagaacaaaat
gaattaattcgagaaagaagtgaaaagagtgtagagataacaaaacag
gataccaaagttgagctggagacttacaagcaaactcggcaaggtctg
gatgaaatgtacagtgatgtgtggaagcagctaaaagaggagaagaaa
gtccggttggaactggaaaaagaactggagttacaaattggaatgaaa
accgaaatggaaattgcaatgaagttactggaaaaggacacccacgag
aagcaggacacactagttgccctccgccagcagctggaagaagtcaaa
gcgattaatttacagatgtttcacaaagctcagaatgcagagagcagt
ttgcagcagaagaatgaagccatcacatcctttgaaggaaaaaccaac
caagttatgtccagcatgaaacaaatggaagaaaggttgcagcactcg
gagcgggcgaggcagggagctgaggagcggagccacaagctgcagcag
gagctgggcgggaggatcggcgccctgcagctgcagctctcccagctg
cacgagcaatgctcaagcctggagaaagaattgaaatcagaaaaagag
caaagacaggctcttcagcgcgaattacagcacgagaaagacacttcc
tctctactcaggatggagctgcaacaagtggaaggactgaaaaaggag
ttgcgggagcncaggacgagaaggcagagctgcagaagatctgcgagg
agcaggaacaagccctccaggaaatgggcctgcacctcagccagtcca
agctgaagatggaagatataaaagaagtgaaccaggcactgaagggcc
acgcctggctgaaagatgacgaagcgacacactgtaggcagtgtgaga
aggagttctccatttcccggagaaagcaccactgccggaactgtggcc
acatcttctgcaacacctgctccagcaacgagctggccctgccctcct
accccaagccggtgcgagtgtgcgacagctgccacaccctgctcctgc
agcgctgctcctccacggcctcctga
Gene symbol: SMN1
Entrez Gene ID: 6606
dna/ac: BC000908.2
sequence:
(SEQ ID NO: 33)
atggcgatgagcagcggcggcagtggtggcggcgtcccggagcaggag
gattccgtgctgttccggcgcggcacaggccagagcgatgattctgac
atttgggatgatacagcactgataaaagcatatgataaagctgtggct
tcatttaagcatgctctaaagaatggtgacatttgtgaaacttcgggt
aaaccaaaaaccacacctaaaagaaaacctgctaagaagaataaaagc
caaaagaagaatactgcagcttccttacaacagtggaaagttggggac
aaatgttctgccatttggtcagaagacggttgcatttacccagctacc
attgtttcaattgattttaagagagaaacctgtgttgtggtttacact
ggatatggaaatagagaggagcaaaatctgtccgatctactttcccca
atctgtgaagtagctaataatatagaacagaatgctcaagagaatgaa
aatgaaagccaagtttcaacagatgaaagtgagaactccaggtctcct
ggaaataaatcagataacatcaagcccaaatctgctccatggaactct
tttctccctccaccaccccccatgccagggccaagactgggaccagga
aagccaggtctaaaattcaatggcccaccaccgccaccgccaccacca
ccaccccacttactatcatgctggctgcctccatttccttctggacca
ccaataattcccccaccacctcccatatgtccagattctcttgatgat
gctgatgctttgggaagtatgttaatttcatggtacatgagtggctat
catactggctattatatggaaatgctggcatag
Gene symbol: SNK
Entrez Gene ID: 10769
dna/ac: BC013879.2
sequence:
(SEQ ID NO: 34)
atggagcttttgcggactatcacctaccagccagccgccagcaccaaa
atgtgcgagcaggcgctgggcaagggttgcggagcagactcgaagaag
aagcggccgccgcagccccccgaggaatcgcagccacctcagtcccag
gcgcaagtgcccccggcggcccctcaccaccatcaccaccattcgcac
tcggggccggagatctcgcggattatcgtcgaccccacgactgggaag
cgctactgccggggcaaagtgctgggaaagggtggattgcaaaatgtt
acgagatgacagatttgacaaataacaaagtctacgccgcaaaaatta
ttcctcacagcagagtagctaaacctcatcaaagggaaaagattgaca
aagaaatagagcttcacagaattcttcatcataagcatgtagtgcagt
tttaccactacttcgaggacaaagaaaacatttacattctcttggaat
actgcagtagaaggtcaatggctcatattttgaaagcaagaaaggtgt
tgacagagccagaagttcgatactacctcaggcagattgtgtctggac
tgaaataccttcatgaacaagaaatcttgcacagagatctcaaactag
ggaacttttttattaatgaagccatggaactaaaagttggggacttcg
gtctggcagccaggctagaacccttggaacacagaaggagaacgatat
gtggtaccccaaattatctctctcctgaagtcctcaacaaacaaggac
atggctgtgaatcagacatttgggccctgggctgtgtaatgtatacaa
tgttactagggaggcccccatttgaaactacaaatctcaaagaaactt
ataggtgcataagggaagcaaggtatacaatgccgtcctcattgctgg
ctcctgccaagcacttaattgctagtatgttgtccaaaaacccagagg
atcgtcccagtttggatgacatcattcgacatgacttttttttgcagg
gcttcactccggacagactgtcttctagctgttgtcatacagttccag
atttccacttatcaagcccagctaagaatttctttaagaaagcagctg
ctgctctttttggtggcaaaaaagacaaagcaagatatattgacacac
ataatagagtgtctaaagaagatgaagacatctacaagataggcatga
tttgaaaaagacttcaataactcagcaacccagcaaacacaggacaga
tgaggagctccagccacctaccaccacagttgccaggtctggaacacc
cgcagtagaaaacaagcagcagattggggatgctatteggatgatagt
cagagggactatggcagctgtagcagcagcagtgaatgccttgaagac
agtaccatgggaagtgttgcagacacagtggcaagggttcttcgggga
tgtctggaaaacatgccggaagctgattgcattcccaaagagcagctg
agcacatcatttcagtgggtcaccaaatgggttgattactctaacaaa
tatggctttgggtaccagctctcagaccacaccgtcggtgtccttttc
aacaatggtgctcacatgagcctcatccagacaaaaaaacagttcact
attacgcagagcttggccaatgctcagttttcccagcaacagatgctc
ctgagcaatttattagtcaagtgacggtgctgaaatacttttctcatt
acatggaggagaacctcatggatggtggagatctgcctagtgttactg
atattcgaagaccteggctctacctccttcagtggctaaaatctgata
aggccctaatgatgctctttaatgatggcacctttcaggtgaatttct
accatgatcatacaaaaatcatcatctgtagccaaaatgaagaatacc
ttctcacctacatcaatgaggataggatatctacaactttcaggctga
caactctgctgatgtctggctgttcatcagaattaaaaaatcgaatgg
aatatgccctgaacatgctcttacaaagatgtaactga
Gene symbol: SRPK1
Entrez Gene ID: 6732
dna/ac: BC038292.1
sequence:
(SEQ ID NO: 35)
atggagcggaaagtgcttgcgctccaggcccgaaagaaaaggaccaag
gccaagaaggacaaagcccaaaggaaatctgaaactcagcaccgaggc
tctgctccccactctgagagtgatctaccagagcaggaagaggagatt
ctgggatctgatgatgatgagcaagaagatcctaatgattattgtaaa
ggaggttatcatcttgtgaaaattggagatctattcaatgggagatac
catgtgatccgaaagttaggctggggacacttttcaacagtatggtta
tcatgggatattcaggggaagaaatttgtggcaatgaaagtagttaaa
agtgctgaacattacactgaaacagcactagatgaaatccggttgctg
aagtcagttcgcaattcagaccctaatgatccaaatagagaaatggtt
gttcaactactagatgactttaaaatatcaggagttaatggaacacat
atctgcatggtatttgaagttttggggcatcatctgctcaagtggatc
atcaaatccaattatcaggggcttccactgccttgtgtcaaaaaaatt
attcagcaagtgttacagggtatgattatttacataccaagtgccgta
tcacccacactgacattaaaccagagaacatcttattgtcagtgaatg
agcagtacattcggaggctggctgcagaagcaacagaatggcagcgat
ctggagctcctccgccttccggatctgcagtcagtactgctccccagc
ctaaaccagctgacaaaatgtcaaagaataagaagaagaaattgaaga
agaagcagaagcgccaggcagaattactagagaagcgaatgcaggaaa
ttgaggaaatggagaaagagtcgggccctgggcaaaaaagaccaaaca
agcaagaagaatcagagagtectgttgaaagaccatgaaagagaaccc
acctaataaaatgacccaagaaaaacttgaagagtcaagtaccattgg
ccaggatcaaacgcttatggaacgtgatacagagggtggtgcagcaga
aattaattgcaatggagtgattgaagtcattaattatactcagaacag
taataatgaaacattgagacataaagaggatctacataatgctaatga
ctgtgatgtccaaaatttgaatcaggaatctagtttcctaagctccca
aaatggagacagcagcacatctcaagaaacagactcttgtacacctat
aacatctgaggtgtcagacaccatggtgtgccagtcttcctcaactgt
aggtcagtcattcagtgaacaacacattagccaacttcaagaaagcat
tcgggcagagataccctgtgaagatgaacaagagcaagaacataacgg
accactggacaacaaaggaaaatccacggctggaaattttcttgttaa
tccccttgagccaaaaaatgcagaaaagctcaaggtgaagattgctga
ccttggaaatgcttgttgggtgcacaaacatttcactgaagatattca
aacaaggcaatatcgttccttggaagttctaatcggatctggctataa
tacccctgctgacatttggagcacggcatgcatggcctttgaactggc
cacaggtgactatttgtttgaacctcattcaggggaagagtacactcg
agatgaagatcacattgcattgatcatagaacttctggggaaggtgcc
tcgcaagctcattgtggcaggaaaatattccaaggaatttttcaccaa
aaaaggtgacctgaaacatatcacgaagctgaaaccttggggcctttt
tgaggttctagtggagaagtatgagtggtcgcaggaagaggcagctgg
cttcacagatttettactgcccatgttggagctgatccctgagaagag
agccactgccgccgagtgtctccggcacccttggcttaactcctaa
Gene symbol: SSA2
Entrez Gene ID: 6738
dna/ac: BC036658.2
sequence:
(SEQ ID NO: 36)
atggaggaatctgtaaaccaaatgcagccactgaatgagaagcagata
gccaattctcaggatggatatgtatggcaagtcactgacatgaatcga
ctacaccggttatatgatcggttctgaaggtgggacttattatatcaa
agaacagaagttgggccttgaaaatgctgaagctttaattagattgat
tgaagatggcagaggatgtgaagtgatacaagaaataaagtcatttag
tcaagaaggcagaaccacaaagcaagagcctatgctctttgcacttgc
catttgttcccagtgctccgacataagcacaaaacaagcagcatttaa
agctgtttctgaagtttgtcgcattcctacccatctctttacttttat
ccagtttaagaaagatctgaaggaaagcatgaaatgtggcatgtgggg
tcgtgccctccggaaggctatagcggactggtacaatgagaaaggtgg
catggcccttgctctggcagttacaaaatataaacagagaaatggctg
gtctcacaaagatctattaagattgtcacatcttaaaccttccagtga
aggacttgcaattgtgaccaaatatattacaaagggctggaaagaagt
tcatgaattgtataaagaaaaagcactctctgtggagactgaaaaatt
attaaagtatctggaggctgtagagaaagtgaagcgcacaagagatga
gctagaagtcattcatctaatagaagaacatagattagttagagaaca
tcttttaacaaatcacttaaagtctaaagaggtatggaaggctttgtt
acaagaaatgccgcttactgcattactaaggaatctaggaaagatgac
tgctaattcagtacttgaaccaggaaattcagaagtatctttagtatg
tgaaaaactgtgtaatgaaaaactattaaaaaaggctcgtatacatcc
atttcatattttgatcgcattagaaacttacaagacaggtcatggtct
cagagggaaactgaagtggcgccctgatgaagaaattttgaaagcatt
ggatgctgctttttataaaacatttaagacagttgaaccaactggaaa
acgtttcttactagctgttgatgtcagtgcttctatgaaccaaagagt
tttgggtagtatactcaacgctagtacagttgctgcagcaatgtgcat
ggttgtcacacgaacagaaaaagattatatgtagttgattttccgatg
aaatggtaccatgtccagtgactacagatatgaccttacaacaggttt
taatggctatgagtcagatcccagcaggtggaactgattgctctcttc
caatgatctgggctcagaagacaaacacacctgctgatgtatcattgt
attcactgataatgagacattgctggaggtgtccatcctgctattgct
ctgagggagtatcgaaagaaaatggatattccagctaaattgattgtt
tgtggaatgacatcaaatggtttcaccattgcagacccagatgataga
ggcatgttggatatgtgcggattgatactggagctctggatgtaattc
gaaatttcacattagatatgatttaa
Gene symbol: SSNA1
Entrez Gene ID: 8636
dna/ac: BC000864.1
sequence:
(SEQ ID NO: 37)
atgacccagcagggcgcggcgctgcagaactacaacaacgagctggtc
aagtgcatagaggagctgtgccagaagcgggaggagctgtgccggcag
atccaggaggaggaggacgagaagcagcggctgcagaatgaggtgagg
cagctgacagagaagctggcccgcgtcaacgagaacctggcacgcaag
attgcctctcgcaacgagttcgaccggaccatcgcggagacggaggcc
gcctacctcaagatcctggagagctcccagactttgctcagcgttctc
aagagggaagctgggaacctgaccaaggctacagccccagaccagaaa
agtagcggcggcagggacagctga
Gene symbol: STAU1
Entrez Gene ID: 6780
dna/ac: BC050432.1
sequence:
(SEQ ID NO: 38)
atgtctcaagttcaagtgcaagttcagaacccatctgctgctctctca
gggagccaaatactgaacaagaaccagtctcttctctcacagcctttg
atgagtattccttctactactagctctctgccctctgaaaatgcaggt
agacccattcaaaactctgattaccctctgcatctattacatccacca
gtgcagctgcagaaagcataacccctactgtagaactaaatgcactgt
gcatgaaacttggaaaaaaaccaatgtataagcctgttgacccttact
ctcggatgcagtccacctataactacaacatgagaggaggtgcttatc
ccccgaggtacttttacccatttccagttccacctttactttatcaag
tggaactttctgtgggaggacagcaatttaatggcaaaggaaagacaa
gacaggctgcgaaacacgatgctgctgccaaagcgttgaggatcctgc
agaatgagcccctgccagagaggctggaggtgaatggaagagaatccg
aagaagaaaatctcaataaatctgaaataagtcaagtgfttgagattg
cacttaaacggaacttgcctgtgaatttcgaggtggcccgggagagtg
gcccaccccacatgaagaactttgtgaccaaggtttcggttggggagt
ttgtgggggaaggtgaagggaaaagcaagaagatttcaaagaaaaatg
ccgccatagctgttcttgaggagctgaagaagttaccgcccctgcctg
cagttgaacgagtaaagcctagaatcaaaaagaaaacaaaacccatag
tcaagccacagacaagcccagaatatggccaggggatcaatccgatta
gccgactggcccagatccagcaggcaaaaaaggagaaggagccagagt
acacgctcctcacagagcgaggcctcccgcgccgcagggagtttgtga
tgcaggtgaaggttggaaaccacactgcagaaggaacgggcaccaaca
agaaggtggccaagcgcaatgcagccgagaacatgctggagatccttg
gtttcaaagtcccgcaggcgcagcccaccaaacccgcactcaagtcag
aggagaagacacccataaagaaaccaggggatggaagaaaagtaacct
tttttgaacctggctctggggatgaaaatgggactagtaataaagagg
atgagttcaggatgccttatctaagtcatcagcagctgcctgctggaa
ttcttcccatggtgcccgaggtcgcccaggctgtaggagttagtcaag
gacatcacaccaaagattttaccagggcagctccgaatcctgccaagg
ccacggtaactgccatgatagcccgagagttgttgtatgggggcacct
cgcccacagccgagaccattttaaagaataacatctcttcaggccacg
taccccatggacctctcacgagaccctctgagcaactggactatcttt
ccagagtccagggattccaggttgaatacaaagacttccccaaaaaca
acaagaacgaatttgtatctcttatcaattgctcctctcagccacctc
tgatcagccatggtatcggcaaggatgtggagtcctgccatgatatgg
ctgcgctgaacatcttaaagttgctgtctgagttggaccaacaaagta
cagagatgccaagaacaggaaacggaccaatgtctgtgtgtgggaggt
gctga
Gene symbol: STK11
Entrez Gene ID: 6794
dna/ac: BC007981.2
sequence:
(SEQ ID NO: 39)
atggaggtggtggacccgcagcagctgggcatgttcacggagggcgag
ctgatgtcggtgggtatggacacgttcatccaccgcatcgactccacc
gaggtcatctaccagccgcgccgcaagcgggccaagctcatcggcaag
tacctgatgggggacctgctgggggaaggctcttacggcaaggtgaag
gaggtgctggactcggagacgctgtgcaggagggccgtcaagatcctc
aagaagaagaagttgcgaaggatccccaacggggaggccaacgtgaag
aaggaaattcaactactgaggaggttacggcacaaaaatgtcatccag
ctggtggatgtgttatacaacgaagagaagcagaaaatgtatatggtg
atggagtactgcgtgtgtggcatgcaggaaatgctggacagcgtgccg
gagaagcgtttcccagtgtgccaggcccacgggtacttctgtcagctg
attgacggcctggagtacctgcatagccagggcattgtgcacaaggac
atcaagccggggaacctgctgctcaccaccggtggcaccctcaaaatc
tccgacctgggcgtggccgaggcactgcacccgttcgcggcggacgac
acctgccggaccagccagggctccccggctttccagccgcccgagatt
gccaacggcctggacaccttctccggcttcaaggtggacatctggtcg
gctggggtcaccctctacaacatcaccacgggtctgtaccccttcgaa
ggggacaacatctacaagttgtttgagaacatcgggaaggggagctac
gccatcccgggcgactgtggccccccgctctctgacctgctgaaaggg
atgcttgagtacgaaccggccaagaggttctccatccggcagatccgg
cagcacagctggttccggaagaaacatcctccggctgaagcaccagtg
cccatcccaccgagcccagacaccaaggaccggtggcgcagcatgact
gtggtgccgtacttggaggacctgcacggcgcggacgaggacgaggac
ctcttcgacatcgaggatgacatcatctacactcaggacttcacggtg
cccggacaggtcccagaagaggaggccagtcacaatggacagcgccgg
ggcctccccaaggccgtgtgtatgaacggcacagaggcggcgcagctg
agcaccaaatccagggcggagggccgggcccccaaccctgcccgcaag
gcctgctccgccagcagcaagatccgccggctgtcggcctgcaagcag
cagtga
Gene symbol: TOM1
Entrez Gene ID: 10043
dna/ac: BC046151.1
sequence:
(SEQ ID NO: 40)
atggactttctcctggggaacccgttcagctctccagtgggacagcgc
atcgagaaagccacagatggctccctgcagagcgaggactgggccctc
aacatggagatctgcgacatcatcaacgagacggaggaaggtcccaaa
gatgccctccgagcagtaaagaagagaatcgtggggaataagaacttc
cacgaggtgatgctggctctcacagtcttagaaacctgtgtcaagaac
tgcgggcaccgcnccacgtgctggtggccagccaggacncgtggagag
tgtgctggtgaggaccatcctgcccaagaacaacccacccaccatcgt
gcatgacaaagtgctcaacctcatccagtcctgggctgacgcgttccg
cagctcgcccgatctgacaggtgtggtcaccatctatgaggacctgcg
gaggaaaggcctggagttccccatgactgacctggacatgctgtcacc
catccacacaccccagaggaccgtgttcaactcagagacacaatcagg
acaggattctgtgggcactgactccagccagcaagaggactctggcca
gcatgctgcccctctgcccgccccgcccatactctccggtgacacgcc
catagcaccaaccccggaacagattgggaagctgcgcagtgagctgga
gatggtgagtgggaacgtgagggtgatgtcggagatgctgacggagct
ggtgcccacccaggccgagcccgcagacctggagctgctgcaggagct
caaccgcacgtgccgagccatgcagcagcgggtcctggagctcatccc
tcagatcgccaatgagcagctgacagaggagctgctcatcgtcaatga
caatctcaacaatgtgttcctgcgccatgaacggtttgaacggttccg
aacaggccagaccaccaaggccccaagtgaggccgagccggcagctga
cctgatcgacatgggccctgacccagcagccaccggcaacctctcatc
ccagctggcaggaatgaacctgggctccagcagtgtgagagctggcct
gcagtctctggaggcctctggtcgactggaagatgagtttgacatgtt
tgcgctgacacggggcagctcactggctgaccaacggaaagaggtaaa
atacgaagccccccaagcaacagacggcctggctggagccctggacgc
ccggcagcagagcactggcgcgatcccagtcacccaggcctgcctcat
ggaggacatcgagcagtggctgtccactgacgtgggtaatgatgcgga
agagcctaagggggtcaccagcgaaggtaaatttgacaaattcctgga
agaacgggccaaagccgcggaccgattgcccaacctctccagcccctc
agctgaggggcccccgggtcccccatctggcccagcgccccggaagaa
gacccaggagaaagatgatgacatgctgtttgccttatga
Gene symbol: TXNL2
Entrez Gene ID: 10539
dna/ac: BC005289
sequence:
(SEQ ID NO: 41)
atggcggcgggggcggctgaggcagctgtagcggccgtggaggaggtc
ggctcagccgggcagtttgaggagctgctgcgcctcaaagccaagtcc
ctccttgtggtccatttctgggcaccatgggctccacagtgtgcacag
atgaacgaagttatggcagagttagctaaagaactccctcaagtttca
tttgtgaagttggaagctgaaggtgttcctgaagtatctgaaaaatat
gaaattagctctgttcccacttttctgtttttcaagaattctcagaaa
atcgaccgattagatggtgcacatgccccagagttgaccaaaaaagtt
cagcgacatgcatctagtggctccttcctacccagcgctaatgaacat
cttaaagaagatctcaaccttcgcttgaagaaattgactcatgctgcc
ccctgcatgctgtttatgaaaggaactcctcaagaaccacgctgtggt
ttcagcaagcagatggtggaaattcttcacaaacataatattcagttt
agcagttttgatatcttctcagatgaagaggttcgacagggactcaaa
gcctattccagttggcctacctatcctcagctctatgtttctggagag
ctcataggaggacttgatataattaaggagctagaagcatctgaagaa
ctagatacaatttgtcccaaagctcccaaattagaggaaaggctcaaa
gtgctgacaaataaagcttctgtgatgctctttatgaaaggaaacaaa
caggaagcaaaatgtggattcagcaaacaaattctggaaatactaaat
agtactggtgttgaatatgaaacattcgatatattggaggatgaagaa
gttcggcaaggattaaaagcttactcaaattggccaacataccctcag
ctgtatgtgaaaggggagctggtgggaggattggatattgtgaaggaa
ctgaaagaaaatggtgaattgctgcctatactgagaggagaaaattaa
Gene symbol: TXNRD1
Entrez Gene ID: 7296
dna/ac: BC018122.1
sequence:
(SEQ ID NO: 42)
atgaacggccctgaagatcttcccaagtcctatgactatgaccttatc
atcattggaggtggctcaggaggtctggcagctgctaaggaggcagcc
caatatggcaagaaggtgatggtcctggactttgtcactcccacccct
cttggaactagatggggtcttggaggaacatgtgtgaatgtgggttgc
atacctaaaaaactgatgcatcaagcagctttgttaggacaagccctg
caagactctcgaaattatggatggaaagtcgaggagacagttaagcat
gattgggacagaatgatagaagctgtacagaatcacattggctctttg
aattggggctaccgagtagctctgcgggagaaaaaagtcgtctatgag
aatgcttatgggcaatttattggtcctcacaggattaaggcaacaaat
aataaaggcaaagaaaaaatttattcagcagagagatttctcattgcc
actggtgaaagaccacgttacttgggcatccctggtgacaaagaatac
tgcatcagcagtgatgatcttttctccttgccttactgcccgggtaag
accctggttgttggagcatcctatgtcgctttggagtgcgctggattt
cttgctggtattggtttagacgtcactgttatggttaggtccattctt
cttagaggatttgaccaggacatggccaacaaaattggtgaacacatg
gaagaacatggcatcaagtttataagacagttcgtaccaattaaagtt
gaacaaattgaagcagggacaccaggccgactcagagtagtagctcag
tccaccaatagtgaggaaatcattgaaggagaatataatacggtgatg
ctggcaataggaagagatgcttgcacaagaaaaattggcttagaaacc
gtaggggtgaagataaatgaaaagactggaaaaatacctgtcacagat
gaagaacagaccaatgtgccttacatctatgccattggcgatatattg
gaggataaggtggagctcaccccagttgcaatccaggcaggaagattg
ctggctcagaggctctatgcaggttccactgtcaagtgtgactatgaa
aatgttccaaccactgtatttactcctttggaatatggtgcttgtggc
ctttctgaggagaaagctgtggagaagtttggggaagaaaatattgag
gtttaccatagttacttttggccattggaatggacgattccgtcaaga
gataacaacaaatgttatgcaaaaataatctgtaatactaaagacaat
gaacgtgttgtgggctttcacgtactgggtccaaatgctggagaagtt
acacaaggctttgcagctgcgctcaaatgtggactgaccaaaaagcag
ctggacagcacaattggaatccaccctgtctgtgcagaggtattcaca
acattgtctgtgaccaagcgctctggggcaagcatcctccaggctggc
tgctgaggttaa
Gene symbol: VCL
Entrez Gene ID: 7414
dna/ac: BC039174.1
sequence:
(SEQ ID NO: 43)
atgccagtgtttcatacgcgcacgatcgagagcatcctggagccggtg
gcacagcagatctcccacctggtgataatgcacgaggagggcgaggtg
gacggcaaagccattcctgacctcaccgcgcccgtggccgccgtgcag
gcggccgtcagcaacctcgtccgggttggaaaagagactgttcaaacc
actgaggatcagattttgaagagagatatgccaccagcatttattaag
gttgagaatgcttgcaccaagcttgtccaggcagctcagatgcttcag
tcagacccttactcagtgcctgctcgagattatctaattgatgggtca
aggggcatcctctctggaacatcagacctgctccttaccttcgatgag
gctgaggtccgtaaaattattagagtttgcaaaggaattttggaatat
cttacagtggcagaggtggtggagactatggaagatttggtcacttac
acaaagaatcttgggccaggaatgactaagatggccaagatgattgac
gagagacagcaggagctcactcaccaggagcaccgagtgatgttggtg
aactcgatgaacaccgtgaaagagttgctgccagttctcatttcagct
atgaagatttttgtaacaactaaaaactcaaaaaaccaaggcatagag
gaagctttaaaaaatcgcaattttactctagaaaaaatgagtgctgaa
attaatgagataattcgtgtgttacaactcacctctgggatgaagatg
cctgggccagcaaggacactgaagccatgaagagagcattggcctcca
tagactccaaactgaaccaggccaaaggttggctccgtgaccctagtg
cctccccaggggatgctggtgagcaggccatcagacagatcttagatg
aagctggaaaagttggtgaactctgtgcaggcaaagaacgcagggaga
ttctgggaacttgcaaaatgctagggcagatgactgatcaagtggctg
acctccgtgccagaggacaaggatcctcaccggtggccatgcagaaag
ctcagcaggtatctcagggtctggatgtgctcacagcaaaagtggaaa
atgcagctcgcaagctggaagccatgaccaactcaaagcagagcattg
caaagaagatcgatgctgctcagaactggcttgcagatccaaatggtg
gaccggaaggagaagagcagattcgaggtgctttggctgaagctcgga
aaatagcagaattatgtgatgatcctaaagaaagagatgacattctac
gttcccttggggaaatatctgctctgacttctaaattagcagatctac
gaagacaggggaaaggagattctccagaggctcgagccttggccaaac
aggtggccacggccctgcagaacctgcagaccaaaaccaaccgggctg
tggccaacagcagaccggccaaagcagctgtacaccttgagggcaaga
ttgagcaagcacagcggtggattgataatcccacagtggatgaccgtg
gagtcggtcaggctgccatccgggggcttgtggccgaagggcatcgtc
tggctaatgttatgatggggccttatcggcaagatcttctcgccaagt
gtgaccgagtggaccagctgacagcccagctggctgacctggctgcca
gaggggaaggggagagtcctcaggcacgagcacttgcatctcagctcc
aagactccttaaaggatctaaaagctcggatgcaggaggccatgactc
aggaagtgtcagatgttttcagcgataccacaactcccatcaagctgt
tggcagtggcagccacggcgcctcctgatgcgcctaacagggaagagg
tatttgatgagagggcagctaactttgaaaaccattcaggaaagcttg
gtgctacggccgagaaggcggctgcggttggtactgctaataaatcaa
cagtggaaggcattcaggcctcagtgaagacggcccgagaactcacac
cccaggtggtctcggctgctcgtatcttacttaggaaccctggaaatc
aagctgcttatgaacattttgagaccatgaagaaccagtggatcgata
atgttgaaaaaatgacagggctggtggacgaagccattgataccaaat
ctctgttggatgcttcagaagaagcaattaaaaaagacctggacaagt
gcaagglagctatggccaacattcagcctcagatgctggttgctgggg
caaccagtattgctcgtcgggccaaccggatcctgctggtggctaaga
gggaggtggagaattccgaggatcccaagttccgtgaggctgtgaaag
ctgcctctgatgaattgagcaaaaccatctccccgatggtgatggatg
caaaagctgtggctggaaacatttccgaccctggactgcaaaagagct
tcctggactcaggatatcggatcctgggagctgtggccaaggtcagag
aagccttccaacctcaggagcctgacttcccgccgcctccaccagacc
ttgaacaactccgactaacagatgagcttgctcctcccaaaccacctc
tgcctgaaggtgaggtccctccacctaggcctccaccaccagaggaaa
aggatgaagagttccctgagcagaaggccggggaggtgattaaccagc
caatgatgatggctgccagacagctccatgatgaagctcgcaaatggt
ccagcaagggcaatgacatcattgcagcagccaagcgcatggctctgc
tgatggctgagatgtctcggctggtaagagggggcagtggtaccaagc
gggcactcattcagtgtgccaaggacatcgccaaggcctcagatgagg
tgactcggttggccaaggaggttgccaagcagtgcacagataaacgga
ttagaaccaacctcttacaggtatgtgagcgaatcccaaccataagca
ccagctcaaaatcctgtccacagtgaaggccaccatgctgggccggac
caacatcagtgatgaggagtctgagcaggccacagagatgctggttca
caatgcccagaacctcatgcagtctgtgaaggagactgtgcgggaagc
tgaagctgcttcaatcaaaattcgaacagatgctggatttacactgcg
ctgggttagaaagactccctggtaccagtag
Gene symbol: ZMAT2
Entrez Gene ID: 153527
dna/ac: BC056668.1
sequence:
(SEQ ID NO: 44)
atggcgtcgggcagcgggacaaaaaacttggactttcgccgaaagtgg
gacaaagatgaatatgagaaactcgccgagaagaggctcacggaagag
agagaaaagaaagatggaaaaccagtgcagcctgtcaagcgagagctt
ttacggcatagggactacaaggtggacgggaatccaagcttgggaaga
caattgtcattaccaagacaacccctcaatctgagatgggaggatatt
actgcaatgtctgtgactgtgtggtgaaggactccatcaactttctgg
atcacattaatggaaagaaacatcagagaaacctgggcatgtctatgc
gtgtggaacgttccaccctggatcaggtgaagaaacgttttgaggtca
acaagaagaagatggaagagaagcagaaggattatgattttgaggaaa
ggatgaaggagctcagagaagaggaggaaaaggccaaagcgtacaaga
aagagaaacagaaggagaagaaaaggagggctgaggaggacttgacat
ttgaggaggacgatgagatggcagctgtgatgggcttctctggctttg
gttccaccaagaagagttactga
Gene symbol: ZNF38
Entrez Gene ID: 7589
dna/ac: BC047309.1
sequence:
(SEQ ID NO: 45)
atgatgaccaaggtactaggcatggccccagttctgggccctaggcct
ccacaggagcaggtggggcctctgatggtaaaagtcgaggagaaagaa
gagaaaggcaagtaccttcctagcctggagatgttccgccagcgcttc
aggcagtttgggtaccatgatacccctggaccccgagaggccctgagc
caactccgggtgctctgctgtgagtggctgaggcccgagatccacacc
aaggagcagatcctggagctactggtgctggagcagttcctgaccatc
ctgccccaggagaccaggcctgggtgcaggagcattgcccggagagcg
ctgaagaggctgtcactctcctcgaagatctggagcgggaactggatg
agccaggacaccaggtctcaactcctccaaacgaacagaaaccggtgt
gggagaagatatcctcctcaggaactgcaaaggaatccccgagcagca
tgcagccacagcccttggagaccagtcacaaatacgagtcttgggggc
ccctgtacatccaagagtctggtgaggagcaggagttcgctcaagatc
caagaaaggtccgagattgcagattgagtacccagcacgaggaatcag
cagatgagcagaaaggactgaagcagaggggctcaaaggggatataat
ttctgtgattatcgccaataaacctgaggccagcttagagaggcagtg
cgtaaaccttgaaatgaaaaaggaacaaaaccccctatcaagaggcag
gctccaagaaaggtagagaatcagttcctactaaacctaccccaggag
agagacgttatatatgtgctgaatgtggcaaagcctttagtaatagct
caaatctcaccaaacacaggagaacacacactggggagaaaccttacg
tgtgcaccaagtgtgggaaagctttcagccacagctcaaacctcacca
ccactacagaacacacttggtggaccggccetatgactgtaagtgtgg
aaaagcttttgggcagagctcagaccttcttaaacatcagagaatgca
cacagaagaggcgccatatcagtgcaaagattgtggcaaggctttcag
cgggaaaggcagcctcattcgtcactatcggatccacactggggagaa
gccttatcagtgtaacgaatgtgggaagagcttcagtcagcatgcggg
cctcagacccaccagagactccacaccggagagaagccatataagtgt
aaggagtgtgggaaagccttcaaccacagctccaacttcaataaacac
cacagaatccacaccggggaaaagccctactggtgtcatcactgtgga
aagaccttctgtagcaagtccaatctttccaaacatcagcgagtccac
actggagagggagaagcaccgtaa

1. A method useful in diagnosing a patient as having or being predisposed to developing lupus, comprising testing a sample from the patient against a panel of auto-antigens to detect the presence, absence or a modulated level of auto-antibodies in the sample, wherein the panel of auto-antigens comprises:
(a) ZMAT2 and the other 31 auto-antigens listed in Table 2,
(b) the panel of (a) in which up to 5 auto-antigens are deleted or replaced, or
(c) the panel of (a) in which up to 6, 7, 8, 9 or 10 or more auto-antigens are deleted or replaced.

2. A method useful in diagnosing a patient as having or being predisposed to developing lupus, comprising testing a sample from the patient against a panel of auto-antigens to detect the presence, absence or a modulated level of auto-antibodies in the sample, wherein the panel of auto-antigens comprises:
(a) the auto-antigens listed in Table 1,
(b) the panel of (a) in which up to 5 auto-antigens are deleted or replaced, or
(c) the panel of (a) in which up to 6, 7, 8, 9 or 10 or more auto-antigens are deleted or replaced.

3. A method useful in diagnosing a patient as having or being predisposed to developing lupus, comprising testing a sample from the patient against a panel of auto-antigens to detect the presence, absence or a modulated level of auto-antibodies in the sample, wherein the panel of auto-antigens comprises:
(a) the auto-antigens listed in Table 4.
(b) the panel of (a) in which up to 5 auto-antigens are deleted or replaced, or
(c) the panel of (a) in which up to 6, 7, 8, 9 or 10 or more auto-antigens are deleted or replaced.

4. A method useful in diagnosing a patient as having or being predisposed to developing lupus, comprising testing a sample from the patient against a panel of auto-antigens to detect the presence, absence or a modulated level of auto-antibodies in the sample, wherein the panel of auto-antigens comprises:
(a) the auto-antigens listed in Table 3,
(b) the panel of (a) in which up to 5 auto-antigens are deleted or replaced, or
(c) the panel of (a) in which up to 10, 15, 20 or 25 or more auto-antigens are deleted or replaced.

5. A method of diagnosing a patient as having or being predisposed to developing lupus, comprising detecting in a sample taken from said patient one or more auto-antibodies that bind an auto-antigen selected from the group consisting of the auto-antigens listed in Table 3, wherein the presence of said one or more auto-antibodies, or an increase in the concentration of said one or more auto-antibodies, indicates that the patient suffers from or is predisposed to develop lupus.

6. A method useful in diagnosing a patient as having or being predisposed to developing lupus, comprising testing a sample from the patient against a panel of auto-antigens to detect the presence, absence or a modulated level of auto-antibodies in the sample, wherein the panel of auto-antigens comprises at least 10 auto-antigens selected from Table 3.

7. A method according to claim 6, wherein the at least 10 auto-antigens are selected from Table 1.

8. A method according to claim 6, wherein the at least 10 auto-antigens are selected from Table 2.

9. A method according to claim 6, wherein the panel comprises at least 20 auto-antigens and/or wherein the panel comprises ZMAT2.

10. A method according to claim 3, wherein the panel comprises one or more additional auto-antigens selected from the group consisting of the auto-antigens of Table 5.

11. The method of claim 1 wherein detecting the presence or an increased level of at least two auto-antibodies provides information useful for diagnosis.

12. The method of claim 11, wherein detecting the presence of an increased level of at least three, preferably at least four and more preferably at least five auto-antibodies provides information useful for diagnosis.

13. The method of claim 1, wherein the sample is a body fluid.

14. The method of claim 13, wherein the body fluid is selected from blood, serum, plasma, saliva, lymphatic fluid, wound secretion, urine, faeces, mucus or cerebrospinal fluid (CSF).

15. (canceled)

16. A method of monitoring the progression or regression of lupus, or monitoring the progression to a flare of the disease or transition from a flare into remission, comprising:
(a) performing the method of claim 1,
(b) repeating step (a) using a second sample obtained from the patient at a time later than the first sample;
(c) comparing the auto-antibodies detected in said first and second samples; wherein a change in the level or presence or absence of the auto-antibodies detected between the first and second samples indicates the progression or regression of lupus, or the progression to a flare, or transition from a flare into remission.

17. A method of monitoring the efficacy in a patient of a therapeutic agent to lupus, comprising:
(a) performing the method of claim 1,
(b) administering the therapeutic agent to the patient;
(c) performing the method of claim 1 using a second sample from the patient taken after the administration of the therapeutic agent;
(d) comparing the auto-antibodies detected in said first and second samples; wherein a change in the level or presence or absence of the auto-antibodies detected in the first and second sample is indicative of the efficacy of the therapeutic agent to lupus.

18. A method of diagnosing a patient as having or being predisposed to developing lupus, comprising detecting in a sample taken from said patient one or more auto-antibodies that bind an auto-antigen selected from:
a) the group consisting of ZMAT2 (Zinc finger, matrin type 2); BPY2IP1 (MAP1S protein); CEBPG (CCAAT/enhancer binding protein (C/EBP), gamma); E1B-AP5 (E1B-55 kDa-associated protein 5); FUS (Fusion (involved in t(12;16) in malignant liposarcoma)); HAGH (Hydroxyacylglutathione hydrolase); HMG20B (High-mobility group 20B); HOXB6 (Romeo box B6, transcript variant 2); KRT8 (Keratin 8); LIN28 (Lin-28 homolog (C. elegans)); NDUFV3 (NADH dehydrogenase (ubiquinone) flavoprotein 3, 10 kDa); PABPC1 (Poly(A) binding protein, cytoplasmic 1); PHLDA1 (Pleckstrin homology-like domain, family A, member 1); PIAS2 (Msx-interacting-zinc finger, transcript variant alpha); RAB11FIP3 (RAB11 family interacting protein 3 (class H)); RAN (RAN, member RAS oncogene family); RARA (Retinoic acid receptor alpha); RBMS1 (RNA binding motif, single, stranded interacting protein 1, transcript variant); RDBP (RD RNA binding protein); RNF12 (Ring finger protein 12, transcript variant 1); RUFY1 (RUN and FYVE domain containing 1); SMN1 (Survival of motor neuron 1, telomeric); SRPK1 (SFRS protein kinase 1); SSNA1 (Sjogren's syndrome nuclear autoantigen 1); STAU (Staufen, RNA binding protein (Drosophila), transcript variant T3); STK11 (Serine/threonine kinase 11 (Peutz-Jeghers syndrome)); TOM1 (Target of myb1 (chicken)); TXNL2 (Thioredoxin-like, clone MGC:12349); TXNRD1 (Thioredoxin reductase 1, transcript variant 5); and ZNF38 (Zinc finger protein 38), or
b) the group consisting of ZMAT2 (Zinc finger, matrin type 2); ASPSCR1 (alveolar soft part sarcoma chromosome region, candidate 1); CEBPG (CCAAT/enhancer binding protein (C/EBP), gamma); DDX55 (DEAD (Asp-Glu-Ala-Asp) box polypeptide 55); DOM3Z (dom-3 homolog Z (C. elegans)); E1B-AP5 (E1B-55 kDa-associated protein 5) FUS (Fusion (involved in t(12;16) in malignant liposarcoma)); HMG20B (High-mobility group 20B); KRT8 (Keratin 8); LIN28 Lin-8 homolog (C. elegans)); LNX (ligand of numb-protein X); MAP1S (MAP1S protein); PABPC1 (Poly(A) binding protein, cytoplasmic 1); PHLDA1 (Pleckstrin homology-like domain, family A, member 1); PIAS2 (Msx-interacting-zinc finger, transcript variant alpha); PRKCBP1 (protein kinase C binding protein 1); PRKRA (protein kinase, interferon-inducible double stranded RNA dependent activator); RARA (Retinoic acid receptor, alpha); RDBP (RD RNA binding protein); RPL30 (ribosomal protein L30); RPL31 (ribosomal protein L31); SNK (serum-inducible kinase); SRPK1 (SFRS protein kinase 1); STAU1 (Staufen, RNA binding protein (Drosophila), transcript variant T3); TXNL2 (Thioredoxin-like, clone MGC:12349); VCL (vinculin); and ZNF38 (Zinc finger protein 38),
wherein the presence of said one or more auto-antibodies, or an increase in the concentration of said one or more auto-antibodies, indicates that the patient suffers from or is predisposed to develop lupus.

19. The method of claim 18, wherein the presence of auto-antibodies to at least two of the auto-antigens are detected.

20. A method of diagnosing a patient as having or being predisposed to developing lupus, comprising detecting in a sample from said patient auto-antibodies to a panel comprising 5 or more auto-antigens selected from:
a) the group consisting of ZMAT2 (Zinc finger, matrin type 2); BPY2IP1 (MAP1S protein); CEBPG (CCAAT/enhancer binding protein (C/EBP), gamma); E1B-AP5 (E1B-55 kDa-associated protein 5); FUS (Fusion (involved in t(12;16) in malignant liposarcoma)); HAGH (Hydroxyacylglutathione hydrolase); HMG20B (High-mobility group 20B); HOXB6 (Homeo box B6, transcript variant 2); KRT8 (Keratin 8); LIN28 (Lin-28 homolog (C. elegans)); NDUFV3 (NADH dehydrogenase (ubiquinone) flavoprotein 3, 10 kDa); PABPC1 (Poly(A) binding protein, cytoplasmic 1); PHLDA1 (Pleckstrin homology-like domain, family A, member 1); PIAS2 (Msx-interacting-zinc finger, transcript variant alpha); RAB11FIP3 (RAB11 family interacting protein 3 (class II)); RAN (RAN, member RAS oncogene family); RARA (Retinoic acid receptor alpha); RBMS1 (RNA binding motif, single stranded interacting protein 1, transcript variant); RDBP (RD RNA binding protein); RNF12 (Ring finger protein 12, transcript variant 1); RUFY1 (RUN and FYVE domain containing 1); SMN1 (Survival of motor neuron 1. telomeric); SRPK1 (SFRS protein kinase 1): SSNA1 (Sjogren's syndrome nuclear autoantigen 1); STAU (Staufen, RNA binding protein (Drosophila), transcript variant T3); STK11 (Serine/threonine kinase 11 (Peutz-Jeghers syndrome)); TOM1 (Target of myb1 (chicken)); TXNL2 (Thioredoxin-like, clone MGC:12349); TXNRD1 (Thioredoxin reductase 1. transcript variant 5); and ZNF38 (Zinc finger protein 38); or
b) the group consisting of ZMAT2 (Zinc finger, matrin type 2); ASPSCR1 (alveolar soft part sarcoma chromosome region, candidate 1); CEBPG (CCAAT/enhancer binding protein (C/EBP), gamma); DDX55 (DEAD (Asp-Glu-Ala-Asp) box polypeptide 55); DOM3Z (dom-3 homolog Z (C. elegans)); E1B-AP5 (E1B-55 kDa-associated protein 5) FUS (Fusion (involved in t(12;16) in malignant liposarcoma)); HMG20B (High-mobility group 20B); KRT8 (Keratin 8); LIN28 Lin-28 homolog (C. elegans)); LNX (ligand of numb-protein X); MAP1S (MAP1S protein); PABPC1 (Poly(A) binding protein, cytoplasmic 1); PHLDA1 (Pleckstrin homology-like domain, family A, member 1); PIAS2 (Msx-interacting-zinc finger, transcript variant alpha); PRKCBP1 (protein kinase C binding protein 1); PRKRA (protein kinase, interferon-inducible double stranded RNA dependent activator); RARA (Retinoic acid receptor, alpha); RDBP (RD RNA binding protein); RPL30 (ribosomal protein L30); RPL31 (ribosomal protein L31); SNK (serum-inducible kinase); SRPK1 (SFRS protein kinase 1); STAU1(Staufen, RNA binding protein (Drosophila), transcript variant T3); TXNL2 (Thioredoxin-like, clone MGC:12349); VCL (vinculin); ZNF38 (Zinc finger protein 38).
wherein the presence or separately a significant increase in the concentration of said auto-antibodies in the sample to substantially all members of the panel indicates that the patient suffers from or is predisposed to develop lupus.

21. The method of claim 20, wherein the panel comprises 10 or more of said auto-antigens.

22-39. (canceled)

40. The method of claim 1, wherein the sample is a body fluid.

41. The method of claim 40, wherein the body fluid is selected from blood, serum, plasma, saliva, lymphatic fluid, wound secretion, urine, faeces, mucus or cerebrospinal fluid (CSF).

42. The method of claim 41, wherein the sample is serum or plasma.

43. A diagnostic kit for use in diagnosing a patient as having or being predisposed to developing lupus, said kit comprising one or more auto-antigens selected from:
(a) the group consisting of ZMAT2 (Zinc finger, matrin type 2); BPY2IP1 (MAP1S protein); CEBPG (CCAAT/enhancer binding protein (C/EBP), gamma); E1B-AP5 (E1B-55 kDa-associated protein 5); FUS (Fusion (involved in t(12;16) in malignant liposarcoma)); HAGH (Hydroxyacylglutathione hydrolase); HMG20B (High-mobility group 20B); HOXB6 (Homeo box B6, transcript variant 2); KRT8 (Keratin 8); LIN28 (Lin-28 homolog (C. elegans)); NDUFV3 (NADH dehydrogenase (ubiquinone) flavoprotein 3, 10 kDa); PABPC1 (Poly(A) binding protein, cytoplasmic 1); PHLDA1 (Pleckstrin homology-like domain, family A, member 1); PIAS2 (Msx-interacting-zinc finger, transcript variant alpha); RAB11FIP3 (RAB11 family interacting protein 3 (class II)); RAN (RAN, member RAS oncogene family); RARA (Retinoic acid receptor alpha); RBMS1 (RNA binding motif, single stranded interacting protein 1, transcript variant); RDBP (RD RNA binding protein); RNF12 (Ring finger protein 12. transcript variant 1); RUFY1 (RUN and FYVE domain containing 1); SMN1 (Survival of motor neuron 1, telomeric); SRPK1 (SFRS protein kinase 1); SSNA1 (Sjogren's syndrome nuclear autoantigen 1); STAU (Staufen, RNA binding protein (Drosophila), transcript variant T3); STK11 (Serine/threonine kinase 11 (Peutz-Jeghers syndrome)); TOM1 (Target of myb1 (chicken)); TXNL2 (Thioredoxin-like, clone MGC:12349); TXNRD1 (Thioredoxin reductase 1, transcript variant 5); ZNF38 (Zinc finger protein 38); and
(b) the group consisting of the auto-antigens of ZMAT2 (Zinc finger, matrin type 2); ASPSCR1 (alveolar soft part sarcoma chromosome region, candidate 1); CEBPG (CCAAT/enhancer binding protein (C/EBP), gamma); DDX55 (DEAD (Asp-Glu-Ala-Asp) box polypeptide 55); DOM3Z (dom-3 homolog Z (C. elegans)); E1B-AP5 (E1B-55 kDa-associated protein 5) FUS (Fusion (involved in t(12; 16) in malignant liposarcoma)); HMG20B (High-mobility group 20B); KRT8 (Keratin 8); LIN28 Lin-28 homolog (C. elegans)); LNX (ligand of numb-protein X); MAP1S (MAP1S protein); PABPC1 (Poly(A) binding protein, cytoplasmic 1); PHLDA1 (Pleckstrin homology-like domain, family A, member 1); PIAS2 (Msx-interacting-zinc finger, transcript variant alpha); PRKCBP1 (protein kinase C binding protein 1); PRKRA (protein kinase, interferon-inducible double stranded RNA dependent activator); RARA (Retinoic acid receptor, alpha); RDBP (RD RNA binding protein); RPL30 (ribosomal protein L30); RPL31 (ribosomal protein L31); SNK (serum-inducible kinase); SRPK1 (SFRS protein kinase 1); STAU1 (Staufen, RNA binding protein (Drosophila), transcript variant T3); TXNL2 (Thioredoxin-like, clone MGC:12349); VCL (vinculin); ZNF38 (Zinc finger protein 38), or
(c) the group consisting of the auto-antigens of Table 3;
which one or more auto-antigens are immobilised on a substrate.

44. A diagnostic kit for use in diagnosing a patient as having or being predisposed to developing lupus, said kit comprising a panel comprising 5 or more auto-antigens selected from:
(a) the group consisting of ZMAT2 (Zinc finger, matrin type 2); BPY21P1 (MAP1S protein); CEBPG (CCAAT/enhancer binding protein (C/EBP), gamma); E1B-AP5 (E1B-55 kDa-associated protein 5); FUS (Fusion (involved in t(12;16) in malignant liposarcoma)); HAGH (Hydroxyacylglutathione hydrolase); HMG20B (High-mobility group 20B); HOXB6 (Homeo box B6, transcript variant 2); KRT8 (Keratin 8); LIN28 (Lin-28 homolog (C. elegans)); NDUFV3 (NADH dehydrogenase (ubiquinone) flavoprotein 3, 10 kDa); PABPC1 (Poly(A) binding protein, cytoplasmic 1); PHLDA1 (Pleckstrin homology-like domain, family A, member 1); PIAS2 (Msx-interacting-zinc finger, transcript variant alpha); RAB11FIP3 (RAB11 family interacting protein 3 (class II)); RAN (RAN, member RAS oncogene family); RARA (Retinoic acid receptor alpha); RBMS1 (RNA binding motif, single stranded interacting protein 1, transcript variant); RDBP (RD RNA binding protein); RNF12 (Ring finger protein 12, transcript variant 1); RUFY1 (RUN and FYVE domain containing 1); SMN1 (Survival of motor neuron 1, telomeric); SRPK1 (SFRS protein kinase 1); SSNA1 (Sjogren's syndrome nuclear autoantigen 1); STAU (Staufen, RNA binding protein (Drosophila), transcript variant T3); STK11 (Serine/threonine kinase 11 (Peutz-Jeghers syndrome)); TOM1 (Target of myb1 (chicken)); TXNL2 (Thioredoxin-like, clone MGC:12349); TXNRD1 (Thioredoxin reductase 1, transcript variant 5); and ZNF38 (Zinc finger protein 38);
(b) the group consisting of the auto-antigens ZMAT2 (Zinc finger, matrin type 2); ASPSCR1 (alveolar soft part sarcoma chromosome region, candidate 1); CEBPG (CCAAT/enhancer binding protein (C/EBP), gamma); DDX55 (DEAD (Asp-Glu-Ala-Asp) box polypeptide 55); DOM3Z (dom-3 homolog Z (C. elegans)); E1B-AP5 (E1B-55 kDa-associated protein 5) FUS (Fusion (involved in t(12; 16) in malignant liposarcoma)); HMG20B (High-mobility group 20B); KRT8 (Keratin 8); LIN28 Lin-28 homolog (C. elegans)); LNX (ligand of numb-protein X); MAP1S (MAP1S protein); PABPC1 (Poly(A) binding protein, cytoplasmic 1); PHLDA1 (Pleckstrin homology-like domain, family A, member 1); PIAS2 (Msx-interacting-zinc finger, transcript variant alpha); PRKCBP1 (protein kinase C binding protein 1); PRKRA (protein kinase, interferon-inducible double stranded RNA dependent activator); RARA (Retinoic acid receptor, alpha); RDBP (RD RNA binding protein); RPL30 (ribosomal protein L30); RPL31 (ribosomal protein L31); SNK (serum-inducible kinase); SRPK1 (SFRS protein kinase 1); STAU1 (Staufen, RNA binding protein (Drosophila), transcript variant T3); TXNL2 (Thioredoxin-like, clone MGC:12349); VCL (vinculin); and ZNF38 (Zinc finger protein 38); or
(c) the group consisting of the auto-antigens of Table 3;
wherein the auto-antigens are immobilised on a substrate.

45-59. (canceled)

60. A panel of auto-antigens, wherein said panel comprises or consists of:
(i) (a) the auto-antigens listed in Table 2,
(b) the panel of (a) in which up to 5 auto-antigens are deleted or replaced, or
(c) the panel of (a) in which up to 6, 7, 8, 9 or 10-ere or more auto-antigens are deleted or replaced;
(ii) (a) the auto-antigens listed in Table 1,
(b) the panel of (a) in which up to 5 auto-antigens are deleted or replaced, or
(c) the panel of (a) in which up to 6, 7, 8, 9 or 10 or more auto-antigens are deleted or replaced;
(iii) (a) the auto-antigens listed in Table 4,
(b) the panel of (a) in which up to 5 auto-antigens are deleted or replaced, or
(c) the panel of (a) in which up to 6, 7, 8, 9 or 10 or more auto-antigens are deleted or replaced; or
(iv) (a) the auto-antigens listed in Table 3,
(b) the panel of (a) in which up to 5 auto-antigens are deleted or replaced, or
(c) the panel of (a) in which up to 10, 15, 20 or 25 or more auto-antigens are deleted or replaced.

61. A panel according to claim 60 (i)(b), claim 60 (i)(c), claim 60 (ii)(b), claim 60 (ii)(c), claim 60 (iii)(b), claim 60 (iii)(c), claim 60 (iv)(b) or claim 60 (iv)(c) or a method according to claim 60 which method specifies a panel, wherein the panel comprises ZMAT2.

62. A panel according to claim 60 wherein the auto-antigens are variants.

63. A method according to claim 1 wherein the absence of said auto-antibodies in the sample indicates that the patient does not have or is not predisposed to developing lupus thereby providing a negative diagnosis.

64. A method according to claim 18 wherein the method further comprises detecting in a sample taken from said patient one or more auto-antibodies that bind an auto-antigen selected from the group consisting of: BANK1 (B-cell scaffold protein with ankyrin repeats 1); IFI16 (Interferon, gamma-inducible protein 16); PSME3 (Proteasome (prosome, macropain) activator subunit 3 (PA28 gamma; Ki), transc); RALBP1 (RalA-binding protein 1); and SSA2 (Sjogren syndrome antigen A2 (60 kDa, ribonucleoprotein autoantigen SS-A/Ro)).
